SEQUENCE LISTING (65 sequences)

SEQ ID NO: 1 - protein, 755 aa, Deinococcus radiodurans
MSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFALESAPNLRALAEVATQTVRELTGFDRVMLYKFAPDATGEVIAEARREGLHAFLGHRFPASDIPAQARALYTRHLLRLTADTRAAAVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWPAGADLAPSAAGLLAISVGEGWSECLVWLRPELRLEVAWGGATPDQAKDDLGPRHSFDTYLEEKRGYAEPWHPGEIEEAQDLRDTLTGALGERLSVIRDLNRALTQSNAEWRQYGFVISHHMQEPVRLISQFAELLTRQPRAQDGSPDSPQTERITGFLLRETSRLRSLTQDLHTYTALLSAPPPVRRPTPLGRVVDDVLQDLEPRIADTGASIEVAPELPVIAADAGLLRDLLLHLIGNALTFGGPEPRIAVRTERQGAGWSIAVSDQGAGIAPEYQERIFLLFQRLGSLDEALGNGLGLPLCRKIAELHGGTLTVESAPGEGSTFRCWLPDAGPLPGAADA

SEQ ID NO: 2 - protein, 504 aa, Deinococcus radiodurans
MSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFAFESAPNLRALAEVATQTVRELTGFDRVMLYKFAPDATGEVIAEARREGLHAFLGHRFPASDIPAQARALYTRHLLRLTADTRAATVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWLAGADLAPSAAGLLAISVGEGWSECLVWLRPELRLEVAWGGATPDQAKDDLGPRHSFDTYLEEKRGYAEPWHPGEIEEAQDLRDTLTGALGE

SEQ ID NO: 3 - protein, 750 aa, synthetic construct
KGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKEFSAGSAGSAGTGMSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFAFESAPNLRALAEVATQTVRELTGFDRVMLYKFAPDATGEVIAEARREGLHAFLGHRFPASDIPAQARALYTRHLLRLTADTRAATVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWLAGADLAPSAAGLLAISVGEGWSECLVWLRPELRLEVAWGGATPDQAKDDLGPRHSFDTYLEEKRGYAEPWHPGEIEEAQDLRDTLTGALGE

SEQ ID NO: 4 - protein, 23 aa, synthetic construct
IATGMVGALLLLLVVALGIGLFM

SEQ ID NO: 5 - protein, 20 aa, synthetic construct
SIVSAVVGILLVVVLGVVFG

SEQ ID NO: 6 - protein, 27 aa, synthetic construct
YFYFIATGMVGALLLLLVVALGIGLFM

SEQ ID NO: 7 - protein, 24 aa, synthetic construct
YFYFSIVSAVVGILLVVVLGVVFG

SEQ ID NO: 8 - protein, 542 aa, synthetic construct
RRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 9 - protein, 603 aa, synthetic construct
SIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 10 - protein, 425 aa, synthetic construct
KMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 11 - protein, 357 aa, synthetic construct
NKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 12 - protein, 368 aa, synthetic construct
KLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 13 - protein, 431 aa, synthetic construct
KYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 14 - protein, 435 aa, synthetic construct
KKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 15 - protein, 403 aa, synthetic construct
RKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 16 - protein, 22 aa, synthetic construct
METDTLLLWVLLLWVPGSTGDS

SEQ ID NO: 17 - protein, 8 aa, synthetic construct
RSRFVKKD

SEQ ID NO: 18 - protein, 8 aa, synthetic construct
SYLANEIL

SEQ ID NO: 19 - protein, 10 aa, synthetic construct
FCYENEVALS

SEQ ID NO: 20 - protein, 1413 aa, synthetic construct
METDTLLLWVLLLWVPGSTGDSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKEFSAGSAGSAGTGMSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFAFESAPNLRALAEVATQTVRELTGFDRVMLYKFAPDATGEVIAEARREGLHAFLGHRFPASDIPAQARALYTRHLLRLTADTRAATVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWLAGADLAPSAAGLLAISVGEGWSECLVWLRPELRLEVAWGGATPDQAKDDLGPRHSFDTYLEEKRGYAEPWHPGEIEEAQDLRDTLTGALGELEIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGASRGGGGSGGGGSGGGGSGGGGSRSRFVKKDSAGSAGSAGSAGSYLANEILWGSAGSAGSAGSAGFCYENEVALS

SEQ ID NO: 21 - protein, 1451 aa, synthetic construct
METDTLLLWVLLLWVPGSTGDSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKEFSAGSAGSAGTGMSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFAFESAPNLRALAEVATQTVRELTGFDRVMLYKFAPDATGEVIAEARREGLHAFLGHRFPASDIPAQARALYTRHLLRLTADTRAATVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWLAGADLAPSAAGLLAISVGEGWSECLVWLRPELRLEVAWGGATPDQAKDDLGPRHSFDTYLEEKRGYAEPWHPGEIEEAQDLRDTLTGALGELESIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPVSRGGGGSGGGGSGGGGSGGGGSRSRFVKKDSAGSAGSAGSAGSYLANEILWGSAGSAGSAGSAGFCYENEVALS

SEQ ID NO: 22 - protein, 1297 aa, synthetic construct
METDTLLLWVLLLWVPGSTGDSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKEFSAGSAGSAGTGMSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFAFESAPNLRALAEVATQTVRELTGFDRVMLYKFAPDATGEVIAEARREGLHAFLGHRFPASDIPAQARALYTRHLLRLTADTRAATVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWLAGADLAPSAAGLLAISVGEGWSECLVWLRPELRLEVAWGGATPDQAKDDLGPRHSFDTYLEEKRGYAEPWHPGEIEEAQDLRDTLTGALGEYFYFSIVSAVVGILLVVVLGVVFGSRKMKSGTKKSDFHSQMAVHKLAKSIPLRRQVTVSADSSASMNSGVLLVRPSRLSSSGTPMLAGVSEYELPEDLRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRRSRGGGGSGGGGSGGGGSGGGGSRSRFVKKDSAGSAGSAGSAGSYLANEILWGSAGSAGSAGSAGFCYENEVALS

SEQ ID NO: 23 - protein, 1228 aa, synthetic construct
METDTLLLWVLLLWVPGSTGDSKGEEDNMAIIKEFMRFKHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKEFSAGSAGSAGTGMSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFAFESAPNLRALAEVATQTVRELTGFDRVMLYKFAPDATGEVIAEARREGLHAFLGHRFPASDIPAQARALYTRHLLRLTADTRAATVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWLAGADLAPSAAGLLAISVGEGWSECLVWLRPELRLEVAWGGATPDQAKDDLGPRHSFDTYLEEKRGYAEPWHPGEIEEAQDLRDTLTGALGEYFYFSIVSAVVGILLVVVLGVVFGSRNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLGSRGGGGSGGGGSGGGGSGGGGSRSRFVKKDSAGSAGSAGSAGSYLANEILWGSAGSAGSAGSAGFCYENEVALS

SEQ ID NO: 24 - protein, 1241 aa, synthetic construct
METDTLLLWVLLLWVPGSTGDSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKEFSAGSAGSAGTGMSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFAFESAPNLRALAEVATQTVRELTGFDRVMLYKFAPDATGEVIAEARREGLHAFLGHRFPASDIPAQARALYTRHLLRLTADTRAATVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWLAGADLAPSAAGLLAISVGEGWSECLVWLRPELRLEVAWGGATPDQAKDDLGPRHSFDTYLEEKRGYAEPWHPGEIEEAQDLRDTLTGALGELEIATGMVGALLLLLVVALGIGLFMSRKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILGSRGGGGSGGGGSGGGGSGGGGSRSRFVKKDSAGSAGSAGSAGSYLANEILWGSAGSAGSAGSAGFCYENEVALS

SEQ ID NO: 25 - protein, 1301 aa, synthetic construct
METDTLLLWVLLLWVPGSTGDSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKEFSAGSAGSAGTGMSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFAFESAPNLRALAEVATQTVRELTGFDRVMLYKFAPDATGEVIAEARREGLHAFLGHRFPASDIPAQARALYTRHLLRLTADTRAATVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWLAGADLAPSAAGLLAISVGEGWSECLVWLRPELRLEVAWGGATPDQAKDDLGPRHSFDTYLEEKRGYAEPWHPGEIEEAQDLRDTLTGALGELESIVSAVVGILLVVVLGVVFGSRKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDVSRGGGGSGGGGSGGGGSGGGGSRSRFVKKDSAGSAGSAGSAGSYLANEILWGSAGSAGSAGSAGFCYENEVALS

SEQ ID NO: 26 - protein, 1306 aa, synthetic construct
METDTLLLWVLLLWVPGSTGDSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKEFSAGSAGSAGTGMSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFAFESAPNLRALAEVATQTVRELTGFDRVMLYKFAPDATGEVIAEARREGLHAFLGHRFPASDIPAQARALYTRHLLRLTADTRAATVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWLAGADLAPSAAGLLAISVGEGWSECLVWLRPELRLEVAWGGATPDQAKDDLGPRHSFDTYLEEKRGYAEPWHPGEIEEAQDLRDTLTGALGEYFYFSIVSAVVGILLVVVLGVVFGSRKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETSRGGGGSGGGGSGGGGSGGGGSRSRFVKKDSAGSAGSAGSAGSYLANEILWGSAGSAGSAGSAGFCYENEVALS

SEQ ID NO: 27 - protein, 1273 aa, synthetic construct
METDTLLLWVLLLWVPGSTGDSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKEFSAGSAGSAGTGMSRDPLPFFPPLYLGGPEITTENCEREPIHIPGSIQPHGALLTADGHSGEVLQMSLNAATFLGQEPTVLRGQTLAALLPEQWPALQAALPPGCPDALQYRATLDWPAAGHLSLTVHRVGELLILEFEPTEAWDSTGPHALRNAMFAFESAPNLRALAEVATQTVRELTGFDRVMLYKFAPDATGEVIAEARREGLHAFLGHRFPASDIPAQARALYTRHLLRLTADTRAATVPLDPVLNPQTNAPTPLGGAVLRATSPMHMQYLRNMGVGSSLSVSVVVGGQLWGLIACHHQTPYVLPPDLRTTLEYLGRLLSLQVQVKEAADVAAFRQSLREHHARVALAAAHSLSPHDTLSDPALDLLGLMRAGGLILRFEGRWQTLGEVPPAPAVDALLAWLETQPGALVQTDALGQLWLAGADLAPSAAGLLAISVGEGWSECLVWLRPELRLEVAWGGATPDQAKDDLGPRHSFDTYLEEKRGYAEPWHPGEIEEAQDLRDTLTGALGELESIVSAVVGILLVVVLGVVFGSRRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPSSRGGGGSGGGGSGGGGSGGGGSRSRFVKKDSAGSAGSAGSAGSYLANEILWGSAGSAGSAGSAGFCYENEVALS

SEQ ID NO: 28 - DNA, 2268 bp, Deinococcus radiodurans
  source 1..2268: mol_type = other DNA
atgagccgggacccgttgcccttttttccaccgctttaccttggtggcccggaaattaccaccgagaactgcgagcgcgagccgattcatattcccggcagcatccagccgcacggcgccctgctcactgccgacgggcacagcggcgaggtgctccagatgagcctcaacgcggccacttttctgggacaggaacccacagtgctgcgcggacagaccctcgccgcactgctgcccgagcagtggcccgcgctgcaagcggccctgccccccggctgccccgacgccctgcaataccgcgcaacgctggactggcctgccgccgggcacctttcgctgacggtgcaccgggtcggcgagttgctgattctggaattcgagccgacggaggcctgggacagcaccgggccgcacgcgctgcgcaacgcgatgttcgcgctcgaaagtgcccccaacctgcgggcgctggccgaggtggcgacccagacggtccgcgagctgacgggctttgaccgggtgatgctctacaaatttgcccccgacgccaccggcgaagtgattgccgaggcccgccgtgaggggctgcacgcctttctgggccaccgttttcccgcgtcggacattccggcgcaggcccgcgcgctctacacccggcacctgctgcgcctgaccgccgacacccgcgccgccgccgtgccgctcgatcccgtcctcaacccgcagacgaatgcgcccaccccgctgggcggcgccgtgctgcgcgccacctcgcccatgcacatgcagtacctgcggaacatgggcgtcgggtcgagcctgtcggtgtcggtggtggtcggcggccagctctggggcctgatcgcctgccaccaccagacgccctacgtgttgccgcccgacctgcgaaccacgctcgaatacctgggccgcttgctgagcctgcaagttcaggtcaaggaagcggcggacgtggcggcctttcgccagagcctgcgggagcaccacgcgcgggtggccctcgcggcggcgcactcgctctcgccgcacgacaccctcagtgacccggcgcttgacctgctgggcctgatgcgggccgggggcctgattctgcgtttcgagggccgctggcagacgttgggtgaagtgccgcctgccccggcggtggacgcgctgctggcgtggctcgaaacccagccgggcgccctggtccagaccgacgcgctgggccaactgtggcccgccggcgccgatctcgcccccagcgcagcgggcctgctcgccatcagcgtgggcgagggctggtcggagtgcctcgtctggctgcggcccgaactgcggctggaggtcgcctggggcggggccactcctgaccaggcgaaagacgacctcgggccgcgccactcattcgacacctacctcgaagaaaaacgcggctacgccgagccctggcatcccggcgaaatcgaggaggcgcaggatctacgtgacacattgaccggggcgctgggcgagcgcctgagcgtgattcgtgacctcaaccgggcgctcacacagtcgaacgccgagtggcggcagtacggcttcgttatcagccaccacatgcaggagccggtgcggctcatctcgcagttcgccgagttgctgacgcgccagccccgcgcccaggacgggtctccggactctccgcagaccgagcgcatcaccggctttctgctgcgcgaaacgtcgcgcctgcgcagcctgacgcaagacctccacacctacaccgcgctgctctcggcaccgccgccggtgcgccgccccacgccgctgggccgcgtggtggacgatgtgctgcaagacctcgaaccccgcattgccgacaccggagcgagcatcgaggtggcgcccgagttgcccgtcatcgctgccgacgctggcctgctgcgcgacctgctgctgcatctgatcggcaacgcgctgacgtttggtggcccggagccgcgtattgccgtaaggaccgaacggcaaggcgcgggttggtctatcgcggtcagtgaccagggcgctggcatcgcgcccgagtatcaggaacgaatctttctgctgtttcagcggctcggttcgctcgatgaggcgctgggcaacggcctgggcctgccgctgtgccgcaagatcgccgaactgcatggcggcaccctgaccgtggagtccgcgccaggcgagggcagcaccttccgttgctggctgcccgatgctgggcctcttccgggagccgccgatgcctga

SEQ ID NO: 29 - DNA, 1512 bp, Deinococcus radiodurans
  source 1..1512: mol_type = other DNA
atgagccgggacccgttgcccttttttccaccgctttaccttggtggcccggaaattaccaccgagaactgcgagcgcgagccgattcatattcccggcagcatccagccgcacggcgccctgctcactgccgacgggcacagcggcgaggtgctccagatgagcctcaacgcggccacttttttgggacaggaacccacagtgctgcgcggacagaccctcgccgcactgctgcccgagcagtggcccgcgctgcaagcggccctgccccccggctgccccgacgccctgcaataccgcgcaacgctggactggcctgccgccgggcacctttcgctgacggtgcaccgggtcggcgagttgctgattctggagttcgagccgacggaggcctgggacagcaccgggccgcacgcgctgcgcaacgcgatgttcgcgttcgaaagtgcccccaacctgcgggcgctggccgaggtggcgacccagacggtccgcgagctgacgggctttgaccgggtgatgctctacaaatttgcccccgacgccaccggcgaagtgattgccgaggcccgccgtgaggggctgcacgcctttctgggccaccgttttcccgcgtcggacattccggcgcaggcccgcgcgctctacacccggcacctgctgcgcctgaccgccgacacccgcgccgccaccgtgccgctcgatcccgtcctcaacccgcagacgaatgcgcccaccccgctgggcggcgccgtgctgcgcgccacctcgcccatgcacatgcagtacctgcggaacatgggcgtcgggtcgagcctgtcggtgtcggtggtggtcggcggccagctctggggcctgatcgcctgccaccaccagacgccctacgtgttgccgcccgacctgcgaaccacgctcgaatacctgggccgcttgctgagcctgcaagttcaggtcaaggaagcggcggacgtggcggcctttcgccagagcctgcgggagcaccacgcgcgggtggccctcgcggcggcgcactcgctctcgccgcacgacaccctcagtgacccggcgcttgacctgctgggcctgatgcgggccgggggcctgattctgcgtttcgagggccgctggcagacgttgggtgaagtgccgcctgccccggcggtggacgcgctgctggcgtggctcgaaacccagccgggcgccctggtccagaccgacgcgctaggccaactgtggctcgccggcgccgatctcgcccccagcgcagcgggcctgctcgccatcagcgtgggcgagggctggtcggagtgcctcgtctggctgcggcccgaactgcggctggaggtcgcctggggcggggccactcctgaccaggcgaaagacgacctcgggccgcgccactcattcgacacctacctcgaagaaaaacgcggctacgccgagccctggcatcccggcgaaatcgaggaggcgcaggatctacgtgacacattgaccggggcgctgggcgag

SEQ ID NO: 30 - DNA, 2250 bp, synthetic construct
  source 1..2250: mol_type = other DNA
aagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggaattcagtgctggtagtgctggtagtgctggcaccggtatgagccgggacccgttgcccttttttccaccgctttaccttggtggcccggaaattaccaccgagaactgcgagcgcgagccgattcatattcccggcagcatccagccgcacggcgccctgctcactgccgacgggcacagcggcgaggtgctccagatgagcctcaacgcggccacttttttgggacaggaacccacagtgctgcgcggacagaccctcgccgcactgctgcccgagcagtggcccgcgctgcaagcggccctgccccccggctgccccgacgccctgcaataccgcgcaacgctggactggcctgccgccgggcacctttcgctgacggtgcaccgggtcggcgagttgctgattctggagttcgagccgacggaggcctgggacagcaccgggccgcacgcgctgcgcaacgcgatgttcgcgttcgaaagtgcccccaacctgcgggcgctggccgaggtggcgacccagacggtccgcgagctgacgggctttgaccgggtgatgctctacaaatttgcccccgacgccaccggcgaagtgattgccgaggcccgccgtgaggggctgcacgcctttctgggccaccgttttcccgcgtcggacattccggcgcaggcccgcgcgctctacacccggcacctgctgcgcctgaccgccgacacccgcgccgccaccgtgccgctcgatcccgtcctcaacccgcagacgaatgcgcccaccccgctgggcggcgccgtgctgcgcgccacctcgcccatgcacatgcagtacctgcggaacatgggcgtcgggtcgagcctgtcggtgtcggtggtggtcggcggccagctctggggcctgatcgcctgccaccaccagacgccctacgtgttgccgcccgacctgcgaaccacgctcgaatacctgggccgcttgctgagcctgcaagttcaggtcaaggaagcggcggacgtggcggcctttcgccagagcctgcgggagcaccacgcgcgggtggccctcgcggcggcgcactcgctctcgccgcacgacaccctcagtgacccggcgcttgacctgctgggcctgatgcgggccgggggcctgattctgcgtttcgagggccgctggcagacgttgggtgaagtgccgcctgccccggcggtggacgcgctgctggcgtggctcgaaacccagccgggcgccctggtccagaccgacgcgctaggccaactgtggctcgccggcgccgatctcgcccccagcgcagcgggcctgctcgccatcagcgtgggcgagggctggtcggagtgcctcgtctggctgcggcccgaactgcggctggaggtcgcctggggcggggccactcctgaccaggcgaaagacgacctcgggccgcgccactcattcgacacctacctcgaagaaaaacgcggctacgccgagccctggcatcccggcgaaatcgaggaggcgcaggatctacgtgacacattgaccggggcgctgggcgag

SEQ ID NO: 31 - DNA, 69 bp, synthetic construct
  source 1..69: mol_type = other DNA
atcgccactgggatggtgggggccctcctcttgctgctggtggtggccctggggatcggcctcttcatg

SEQ ID NO: 32 - DNA, 60 bp, synthetic construct
  source 1..60: mol_type = other DNA
tccatcgtctctgcggtggttggcattctgctggtcgtggtcttgggggtggtctttggg

SEQ ID NO: 33 - DNA, 81 bp, synthetic construct
  source 1..81: mol_type = other DNA
tacttttatttcatcgccactgggatggtgggggccctcctcttgctgctggtggtggccctggggatcggcctcttcatg

SEQ ID NO: 34 - DNA, 72 bp, synthetic construct
  source 1..72: mol_type = other DNA
tacttttatttctccatcgtctctgcggtggttggcattctgctggtcgtggtcttgggggtggtctttggg

SEQ ID NO: 35 - DNA, 1626 bp, synthetic construct
  source 1..1626: mol_type = other DNA
cgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcaactcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagca

SEQ ID NO: 36 - DNA, 1809 bp, synthetic construct
  source 1..1809: mol_type = other DNA
tccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtg

SEQ ID NO: 37 - DNA, 1275 bp, synthetic construct
  source 1..1275: mol_type = other DNA
aagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccttcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgc

SEQ ID NO: 38 - DNA, 1071 bp, synthetic construct
  source 1..1071: mol_type = other DNA
aacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccaacgtgaggctgagctgctcaccatgctgcagcaccagcacatcgtgcgcttcttcggcgtctgcaccgagggccgccccctgctcatggtcttcgagtatatgcggcacggggacctcaaccgcttcctccgatcccatggacccgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggc

SEQ ID NO: 39 - DNA, 1107 bp, synthetic construct
  source 1..1107: mol_type = other DNA
aagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggctag

SEQ ID NO: 40 - DNA, 1293 bp, synthetic construct
  source 1..1293: mol_type = other DNA
aaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtc

SEQ ID NO: 41 - DNA, 1305 bp, synthetic construct
  source 1..1305: mol_type = other DNA
aaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatca

SEQ ID NO: 42 - DNA, 1212 bp, synthetic construct
  source 1..1212: mol_type = other DNA
agaaagaggcagccagatgggccgctgggaccgctttacgcttcttcaaaccctgagtatctcagtgccagtgatgtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcacgtggtgcgcctcctgggagtggtgtccaagggccagcccacgctggtggtgatggagctgatggctcacggagacctgaagagctacctccgttctctgcggccagaggctgagaataatcctggccgccctccccctacccttcaagagatgattcagatggcggcagagattgctgacgggatggcctacctgaacgccaagaagtttgtgcatcgggacctggcagcgagaaactgcatggtcgcccatgattttactgtcaaaattggagactttggaatgaccagagacatctatgaaacggattactaccggaaagggggcaagggtctgctccctgtacggtggatggcaccggagtccctgaaggatggggtcttcaccacttcttctgacatgtggtcctttggcgtggtcctttgggaaatcaccagcttggcagaacagccttaccaaggcctgtctaatgaacaggtgttgaaatttgtcatggatggagggtatctggatcaacccgacaactgtccagagagagtcactgacctcatgcgcatgtgctggcaattcaaccccaagatgaggccaaccttcctggagattgtcaacctgctcaaggacgacctgcaccccagctttccagaggtgtcgttcttccacagcgaggagaacaaggctcccgagagtgaggagctggagatggagtttgaggacatggagaatgtgcccctggaccgttcctcgcactgtcagagggaggaggcggggggccgggatggagggtcctcgctgggtttcaagcggagctacgaggaacacatcccttacacacacatgaacggaggcaagaaaaacgggcggattctgaccttgcctcggtccaatccttcctaa

SEQ ID NO: 43 - DNA, 66 bp, synthetic construct
  source 1..66: mol_type = other DNA
atggagacagacacactcctgctatgggtactgctgctctgggttccaggttccactggtgacagc

SEQ ID NO: 44 - DNA, 24 bp, synthetic construct
  source 1..24: mol_type = other DNA
cgcagccgctttgtgaaaaaagat

SEQ ID NO: 45 - DNA, 27 bp, synthetic construct
  source 1..27: mol_type = other DNA
agctatctggcgaacgaaattctgtgg

SEQ ID NO: 46 - DNA, 30 bp, synthetic construct
  source 1..30: mol_type = other DNA
ttttgctatgaaaacgaagtggcgctgtca

SEQ ID NO: 47 - DNA, 4260 bp, synthetic construct
  source 1..4260: mol_type = other DNA
atggagacagacacactcctgctatgggtactgctgctctgggttccaggttccactggtgacagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggaattcagtgctggtagtgctggtagtgctggcaccggtatgagccgggacccgttgcccttttttccaccgctttaccttggtggcccggaaattaccaccgagaactgcgagcgcgagccgattcatattcccggcagcatccagccgcacggcgccctgctcactgccgacgggcacagcggcgaggtgctccagatgagcctcaacgcggccacttttttgggacaggaacccacagtgctgcgcggacagaccctcgccgcactgctgcccgagcagtggcccgcgctgcaagcggccctgccccccggctgccccgacgccctgcaataccgcgcaacgctggactggcctgccgccgggcacctttcgctgacggtgcaccgggtcggcgagttgctgattctggagttcgagccgacggaggcctgggacagcaccgggccgcacgcgctgcgcaacgcgatgttcgcgttcgaaagtgcccccaacctgcgggcgctggccgaggtggcgacccagacggtccgcgagctgacgggctttgaccgggtgatgctctacaaatttgcccccgacgccaccggcgaagtgattgccgaggcccgccgtgaggggctgcacgcctttctgggccaccgttttcccgcgtcggacattccggcgcaggcccgcgcgctctacacccggcacctgctgcgcctgaccgccgacacccgcgccgccaccgtgccgctcgatcccgtcctcaacccgcagacgaatgcgcccaccccgctgggcggcgccgtgctgcgcgccacctcgcccatgcacatgcagtacctgcggaacatgggcgtcgggtcgagcctgtcggtgtcggtggtggtcggcggccagctctggggcctgatcgcctgccaccaccagacgccctacgtgttgccgcccgacctgcgaaccacgctcgaatacctgggccgcttgctgagcctgcaagttcaggtcaaggaagcggcggacgtggcggcctttcgccagagcctgcgggagcaccacgcgcgggtggccctcgcggcggcgcactcgctctcgccgcacgacaccctcagtgacccggcgcttgacctgctgggcctgatgcgggccgggggcctgattctgcgtttcgagggccgctggcagacgttgggtgaagtgccgcctgccccggcggtggacgcgctgctggcgtggctcgaaacccagccgggcgccctggtccagaccgacgcgctaggccaactgtggctcgccggcgccgatctcgcccccagcgcagcgggcctgctcgccatcagcgtgggcgagggctggtcggagtgcctcgtctggctgcggcccgaactgcggctggaggtcgcctggggcggggccactcctgaccaggcgaaagacgacctcgggccgcgccactcattcgacacctacctcgaagaaaaacgcggctacgccgagccctggcatcccggcgaaatcgaggaggcgcaggatctacgtgacacattgaccggggcgctgggcgagctcgagatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcaactcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatctagaggcggtggtggcagcggaggaggggggagcggcggcggtggcagtggcggcggcggtagtcgcagccgctttgtgaaaaaagatagcgccggcagtgcgggtagtgcggggagcgccggtagctatctggcgaacgaaattctgtggggcagcgctggtagcgcaggaagcgccggcagtgcagggttttgctatgaaaacgaagtggcgctgtcataatcagccataccacatttg

SEQ ID NO: 48 - DNA, 4374 bp, synthetic construct
  source 1..4374: mol_type = other DNA
atggagacagacacactcctgctatgggtactgctgctctgggttccaggttccactggtgacagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggaattcagtgctggtagtgctggtagtgctggcaccggtatgagccgggacccgttgcccttttttccaccgctttaccttggtggcccggaaattaccaccgagaactgcgagcgcgagccgattcatattcccggcagcatccagccgcacggcgccctgctcactgccgacgggcacagcggcgaggtgctccagatgagcctcaacgcggccacttttttgggacaggaacccacagtgctgcgcggacagaccctcgccgcactgctgcccgagcagtggcccgcgctgcaagcggccctgccccccggctgccccgacgccctgcaataccgcgcaacgctggactggcctgccgccgggcacctttcgctgacggtgcaccgggtcggcgagttgctgattctggagttcgagccgacggaggcctgggacagcaccgggccgcacgcgctgcgcaacgcgatgttcgcgttcgaaagtgcccccaacctgcgggcgctggccgaggtggcgacccagacggtccgcgagctgacgggctttgaccgggtgatgctctacaaatttgcccccgacgccaccggcgaagtgattgccgaggcccgccgtgaggggctgcacgcctttctgggccaccgttttcccgcgtcggacattccggcgcaggcccgcgcgctctacacccggcacctgctgcgcctgaccgccgacacccgcgccgccaccgtgccgctcgatcccgtcctcaacccgcagacgaatgcgcccaccccgctgggcggcgccgtgctgcgcgccacctcgcccatgcacatgcagtacctgcggaacatgggcgtcgggtcgagcctgtcggtgtcggtggtggtcggcggccagctctggggcctgatcgcctgccaccaccagacgccctacgtgttgccgcccgacctgcgaaccacgctcgaatacctgggccgcttgctgagcctgcaagttcaggtcaaggaagcggcggacgtggcggcctttcgccagagcctgcgggagcaccacgcgcgggtggccctcgcggcggcgcactcgctctcgccgcacgacaccctcagtgacccggcgcttgacctgctgggcctgatgcgggccgggggcctgattctgcgtttcgagggccgctggcagacgttgggtgaagtgccgcctgccccggcggtggacgcgctgctggcgtggctcgaaacccagccgggcgccctggtccagaccgacgcgctaggccaactgtggctcgccggcgccgatctcgcccccagcgcagcgggcctgctcgccatcagcgtgggcgagggctggtcggagtgcctcgtctggctgcggcccgaactgcggctggaggtcgcctggggcggggccactcctgaccaggcgaaagacgacctcgggccgcgccactcattcgacacctacctcgaagaaaaacgcggctacgccgagccctggcatcccggcgaaatcgaggaggcgcaggatctacgtgacacattgaccggggcgctgggcgagctcgagtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtctagaggcggtggtggcagcggaggaggggggagcggcggcggtggcagtggcggcggcggtagtcgcagccgctttgtgaaaaaagatagcgccggcagtgcgggtagtgcggggagcgccggtagctatctggcgaacgaaattctgtggggcagcgctggtagcgcaggaagcgccggcagtgcagggttttgctatgaaaacgaagtggcgctgtcataatcagccataccacatttg

SEQ ID NO: 49 - DNA, 3912 bp, synthetic construct
  source 1..3912: mol_type = other DNA
atggagacagacacactcctgctatgggtactgctgctctgggttccaggttccactggtgacagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggaattcagtgctggtagtgctggtagtgctggcaccggtatgagccgggacccgttgcccttttttccaccgctttaccttggtggcccggaaattaccaccgagaactgcgagcgcgagccgattcatattcccggcagcatccagccgcacggcgccctgctcactgccgacgggcacagcggcgaggtgctccagatgagcctcaacgcggccacttttttgggacaggaacccacagtgctgcgcggacagaccctcgccgcactgctgcccgagcagtggcccgcgctgcaagcggccctgccccccggctgccccgacgccctgcaataccgcgcaacgctggactggcctgccgccgggcacctttcgctgacggtgcaccgggtcggcgagttgctgattctggagttcgagccgacggaggcctgggacagcaccgggccgcacgcgctgcgcaacgcgatgttcgcgttcgaaagtgcccccaacctgcgggcgctggccgaggtggcgacccagacggtccgcgagctgacgggctttgaccgggtgatgctctacaaatttgcccccgacgccaccggcgaagtgattgccgaggcccgccgtgaggggctgcacgcctttctgggccaccgttttcccgcgtcggacattccggcgcaggcccgcgcgctctacacccggcacctgctgcgcctgaccgccgacacccgcgccgccaccgtgccgctcgatcccgtcctcaacccgcagacgaatgcgcccaccccgctgggcggcgccgtgctgcgcgccacctcgcccatgcacatgcagtacctgcggaacatgggcgtcgggtcgagcctgtcggtgtcggtggtggtcggcggccagctctggggcctgatcgcctgccaccaccagacgccctacgtgttgccgcccgacctgcgaaccacgctcgaatacctgggccgcttgctgagcctgcaagttcaggtcaaggaagcggcggacgtggcggcctttcgccagagcctgcgggagcaccacgcgcgggtggccctcgcggcggcgcactcgctctcgccgcacgacaccctcagtgacccggcgcttgacctgctgggcctgatgcgggccgggggcctgattctgcgtttcgagggccgctggcagacgttgggtgaagtgccgcctgccccggcggtggacgcgctgctggcgtggctcgaaacccagccgggcgccctggtccagaccgacgcgctaggccaactgtggctcgccggcgccgatctcgcccccagcgcagcgggcctgctcgccatcagcgtgggcgagggctggtcggagtgcctcgtctggctgcggcccgaactgcggctggaggtcgcctggggcggggccactcctgaccaggcgaaagacgacctcgggccgcgccactcattcgacacctacctcgaagaaaaacgcggctacgccgagccctggcatcccggcgaaatcgaggaggcgcaggatctacgtgacacattgaccggggcgctgggcgagtacttttatttctccatcgtctctgcggtggttggcattctgctggtcgtggtcttgggggtggtctttgggtctagaaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccttcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctctagaggcggtggtggcagcggaggaggggggagcggcggcggtggcagtggcggcggcggtagtcgcagccgctttgtgaaaaaagatagcgccggcagtgcgggtagtgcggggagcgccggtagctatctggcgaacgaaattctgtggggcagcgctggtagcgcaggaagcgccggcagtgcagggttttgctatgaaaacgaagtggcgctgtcataatcagccataccacatttg

SEQ ID NO: 50 - DNA, 3708 bp, synthetic construct
  source 1..3708: mol_type = other DNA
atggagacagacacactcctgctatgggtactgctgctctgggttccaggttccactggtgacagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggaattcagtgctggtagtgctggtagtgctggcaccggtatgagccgggacccgttgcccttttttccaccgctttaccttggtggcccggaaattaccaccgagaactgcgagcgcgagccgattcatattcccggcagcatccagccgcacggcgccctgctcactgccgacgggcacagcggcgaggtgctccagatgagcctcaacgcggccacttttttgggacaggaacccacagtgctgcgcggacagaccctcgccgcactgctgcccgagcagtggcccgcgctgcaagcggccctgccccccggctgccccgacgccctgcaataccgcgcaacgctggactggcctgccgccgggcacctttcgctgacggtgcaccgggtcggcgagttgctgattctggagttcgagccgacggaggcctgggacagcaccgggccgcacgcgctgcgcaacgcgatgttcgcgttcgaaagtgcccccaacctgcgggcgctggccgaggtggcgacccagacggtccgcgagctgacgggctttgaccgggtgatgctctacaaatttgcccccgacgccaccggcgaagtgattgccgaggcccgccgtgaggggctgcacgcctttctgggccaccgttttcccgcgtcggacattccggcgcaggcccgcgcgctctacacccggcacctgctgcgcctgaccgccgacacccgcgccgccaccgtgccgctcgatcccgtcctcaacccgcagacgaatgcgcccaccccgctgggcggcgccgtgctgcgcgccacctcgcccatgcacatgcagtacctgcggaacatgggcgtcgggtcgagcctgtcggtgtcggtggtggtcggcggccagctctggggcctgatcgcctgccaccaccagacgccctacgtgttgccgcccgacctgcgaaccacgctcgaatacctgggccgcttgctgagcctgcaagttcaggtcaaggaagcggcggacgtggcggcctttcgccagagcctgcgggagcaccacgcgcgggtggccctcgcggcggcgcactcgctctcgccgcacgacaccctcagtgacccggcgcttgacctgctgggcctgatgcgggccgggggcctgattctgcgtttcgagggccgctggcagacgttgggtgaagtgccgcctgccccggcggtggacgcgctgctggcgtggctcgaaacccagccgggcgccctggtccagaccgacgcgctaggccaactgtggctcgccggcgccgatctcgcccccagcgcagcgggcctgctcgccatcagcgtgggcgagggctggtcggagtgcctcgtctggctgcggcccgaactgcggctggaggtcgcctggggcggggccactcctgaccaggcgaaagacgacctcgggccgcgccactcattcgacacctacctcgaagaaaaacgcggctacgccgagccctggcatcccggcgaaatcgaggaggcgcaggatctacgtgacacattgaccggggcgctgggcgagtacttttatttctccatcgtctctgcggtggttggcattctgctggtcgtggtcttgggggtggtctttgggtctagaaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccaacgtgaggctgagctgctcaccatgctgcagcaccagcacatcgtgcgcttcttcggcgtctgcaccgagggccgccccctgctcatggtcttcgagtatatgcggcacggggacctcaaccgcttcctccgatcccatggacccgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctctagaggcggtggtggcagcggaggaggggggagcggcggcggtggcagtggcggcggcggtagtcgcagccgctttgtgaaaaaagatagcgccggcagtgcgggtagtgcggggagcgccggtagctatctggcgaacgaaattctgtggggcagcgctggtagcgcaggaagcgccggcagtgcagggttttgctatgaaaacgaagtggcgctgtcataatcagccataccacatttg

SEQ ID NO: 51 - DNA, 3744 bp, synthetic construct
  source 1..3744: mol_type = other DNA
atggagacagacacactcctgctatgggtactgctgctctgggttccaggttccactggtgacagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggaattcagtgctggtagtgctggtagtgctggcaccggtatgagccgggacccgttgcccttttttccaccgctttaccttggtggcccggaaattaccaccgagaactgcgagcgcgagccgattcatattcccggcagcatccagccgcacggcgccctgctcactgccgacgggcacagcggcgaggtgctccagatgagcctcaacgcggccacttttttgggacaggaacccacagtgctgcgcggacagaccctcgccgcactgctgcccgagcagtggcccgcgctgcaagcggccctgccccccggctgccccgacgccctgcaataccgcgcaacgctggactggcctgccgccgggcacctttcgctgacggtgcaccgggtcggcgagttgctgattctggagttcgagccgacggaggcctgggacagcaccgggccgcacgcgctgcgcaacgcgatgttcgcgttcgaaagtgcccccaacctgcgggcgctggccgaggtggcgacccagacggtccgcgagctgacgggctttgaccgggtgatgctctacaaatttgcccccgacgccaccggcgaagtgattgccgaggcccgccgtgaggggctgcacgcctttctgggccaccgttttcccgcgtcggacattccggcgcaggcccgcgcgctctacacccggcacctgctgcgcctgaccgccgacacccgcgccgccaccgtgccgctcgatcccgtcctcaacccgcagacgaatgcgcccaccccgctgggcggcgccgtgctgcgcgccacctcgcccatgcacatgcagtacctgcggaacatgggcgtcgggtcgagcctgtcggtgtcggtggtggtcggcggccagctctggggcctgatcgcctgccaccaccagacgccctacgtgttgccgcccgacctgcgaaccacgctcgaatacctgggccgcttgctgagcctgcaagttcaggtcaaggaagcggcggacgtggcggcctttcgccagagcctgcgggagcaccacgcgcgggtggccctcgcggcggcgcactcgctctcgccgcacgacaccctcagtgacccggcgcttgacctgctgggcctgatgcgggccgggggcctgattctgcgtttcgagggccgctggcagacgttgggtgaagtgccgcctgccccggcggtggacgcgctgctggcgtggctcgaaacccagccgggcgccctggtccagaccgacgcgctaggccaactgtggctcgccggcgccgatctcgcccccagcgcagcgggcctgctcgccatcagcgtgggcgagggctggtcggagtgcctcgtctggctgcggcccgaactgcggctggaggtcgcctggggcggggccactcctgaccaggcgaaagacgacctcgggccgcgccactcattcgacacctacctcgaagaaaaacgcggctacgccgagccctggcatcccggcgaaatcgaggaggcgcaggatctacgtgacacattgaccggggcgctgggcgagctcgagatcgccactgggatggtgggggccctcctcttgctgctggtggtggccctggggatcggcctcttcatgtctagaaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggctctagaggcggtggtggcagcggaggaggggggagcggcggcggtggcagtggcggcggcggtagtcgcagccgctttgtgaaaaaagatagcgccggcagtgcgggtagtgcggggagcgccggtagctatctggcgaacgaaattctgtggggcagcgctggtagcgcaggaagcgccggcagtgcagggttttgctatgaaaacgaagtggcgctgtcataatcagccataccacatttg

SEQ ID NO: 52 - DNA, 3924 bp, synthetic construct
  source 1..3924: mol_type = other DNA
atggagacagacacactcctgctatgggtactgctgctctgggttccaggttccactggtgacagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggaattcagtgctggtagtgctggtagtgctggcaccggtatgagccgggacccgttgcccttttttccaccgctttaccttggtggcccggaaattaccaccgagaactgcgagcgcgagccgattcatattcccggcagcatccagccgcacggcgccctgctcactgccgacgggcacagcggcgaggtgctccagatgagcctcaacgcggccacttttttgggacaggaacccacagtgctgcgcggacagaccctcgccgcactgctgcccgagcagtggcccgcgctgcaagcggccctgccccccggctgccccgacgccctgcaataccgcgcaacgctggactggcctgccgccgggcacctttcgctgacggtgcaccgggtcggcgagttgctgattctggagttcgagccgacggaggcctgggacagcaccgggccgcacgcgctgcgcaacgcgatgttcgcgttcgaaagtgcccccaacctgcgggcgctggccgaggtggcgacccagacggtccgcgagctgacgggctttgaccgggtgatgctctacaaatttgcccccgacgccaccggcgaagtgattgccgaggcccgccgtgaggggctgcacgcctttctgggccaccgttttcccgcgtcggacattccggcgcaggcccgcgcgctctacacccggcacctgctgcgcctgaccgccgacacccgcgccgccaccgtgccgctcgatcccgtcctcaacccgcagacgaatgcgcccaccccgctgggcggcgccgtgctgcgcgccacctcgcccatgcacatgcagtacctgcggaacatgggcgtcgggtcgagcctgtcggtgtcggtggtggtcggcggccagctctggggcctgatcgcctgccaccaccagacgccctacgtgttgccgcccgacctgcgaaccacgctcgaatacctgggccgcttgctgagcctgcaagttcaggtcaaggaagcggcggacgtggcggcctttcgccagagcctgcgggagcaccacgcgcgggtggccctcgcggcggcgcactcgctctcgccgcacgacaccctcagtgacccggcgcttgacctgctgggcctgatgcgggccgggggcctgattctgcgtttcgagggccgctggcagacgttgggtgaagtgccgcctgccccggcggtggacgcgctgctggcgtggctcgaaacccagccgggcgccctggtccagaccgacgcgctaggccaactgtggctcgccggcgccgatctcgcccccagcgcagcgggcctgctcgccatcagcgtgggcgagggctggtcggagtgcctcgtctggctgcggcccgaactgcggctggaggtcgcctggggcggggccactcctgaccaggcgaaagacgacctcgggccgcgccactcattcgacacctacctcgaagaaaaacgcggctacgccgagccctggcatcccggcgaaatcgaggaggcgcaggatctacgtgacacattgaccggggcgctgggcgagctcgagtccatcgtctctgcggtggttggcattctgctggtcgtggtcttgggggtggtctttgggtctagaaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctctagaggcggtggtggcagcggaggaggggggagcggcggcggtggcagtggcggcggcggtagtcgcagccgctttgtgaaaaaagatagcgccggcagtgcgggtagtgcggggagcgccggtagctatctggcgaacgaaattctgtggggcagcgctggtagcgcaggaagcgccggcagtgcagggttttgctatgaaaacgaagtggcgctgtcataatcagccataccacatttg

SEQ ID NO: 53 - protein, 21 aa, synthetic construct
METDTLLLWVLLLWVPGSTGD

SEQ ID NO: 54 - protein, 12 aa, synthetic construct
ANSFCYENEVAL

SEQ ID NO: 55 - protein, 10 aa, synthetic construct
AEKMDIDTGR

SEQ ID NO: 56 - protein, 15 aa, synthetic construct
KLFYKAQRSIWGKKQ

SEQ ID NO: 57 - protein, 12 aa, synthetic construct
ANSFCYENEVAL

SEQ ID NO: 58 - protein, 20 aa, synthetic construct
KSRITSEGEYIPLDQIDINV

SEQ ID NO: 59 - protein, 18 aa, synthetic construct
RSRFVKKDGHCNVQFINV

SEQ ID NO: 60 - protein, 42 aa, synthetic construct
RSRFVKKDSAGSAGSAGSAGSYLANEILWGSAGSAGSAGSAG

SEQ ID NO: 61 - protein, 6 aa, synthetic construct
YFYFYF

SEQ ID NO: 62 - protein, 4 aa, synthetic construct
YFYF

SEQ ID NO: 63 - protein, 22 aa, synthetic construct
YFSIVSAWGILLVWLGWFGILI

SEQ ID NO: 64 - protein, 24 aa, synthetic construct
YFYFSIVSAWGILLVWLGWFGILI

SEQ ID NO: 65 - protein, 26 aa, synthetic construct
YFYFYFSIVSAWGILLVWLGWFGILI

(19) United States
(12) Patent Application Publication
(10) Pub. No.: US 2025/0051398 A1
(43) Pub. Date: Feb. 13, 2025

(54) FUSION CONSTRUCTS OPTICALLY CONTROLLABLE BY FAR RED LIGHT AND METHODS OF USE THEREOF

(71) Applicants: ALBERT EINSTEIN COLLEGE OF MEDICINE, Bronx, NY (US); University of Helsinki, Helsingin Yliopisto (FI)

(72) Inventors: Vladislav V. Verkhusha, Bronx, NY (US); Anna V. Leopold, Helsinki (FI)

(21) Appl. No.: 18/718,699

(22) PCT Filed: Dec. 13, 2022
(86) PCT No.: PCT/US2022/081405

§ 371 Date: Jun. 11, 2024

Related U.S. Application Data
(60) Provisional application No. 63/288,788, filed on Dec. 13, 2021.

(51) Int. Cl. C07K 14/195; C07K 14/71; C12N 13/00

(52) U.S. Cl. CPC C07K 14/195; C07K 14/71; C12N 13/00; C07K 2319/03; C07K 2319/60

(57) ABSTRACT

This disclosure provides a generalized approach for engineering receptor tyrosine kinases (RTKs) optically controlled with far-red light, named eDrRTKs, by targeting a bacterial phytochrome (e.g., DrBphP) to the cell surface and allowing its light-induced conformational changes to be transmitted across the plasma membrane via transmembrane helices to intracellular RTK domains. The ability to activate eDrRTKs with far-red light enabled cross-talk free spectral multiplexing with fluorescent probes operating in a shorter spectral range, allowing for all-optical assays, including non-invasive stimulation in the brain of a live animal. The disclosed engineering approach can be applied beyond RTKs to any membrane receptors, channels, surface antigens, or membrane antibodies that share high similarity with RTKs in mechanisms of their activation.

### CROSS-REFERENCE TO RELATED APPLICATIONS

This application claims the priority and benefit of U.S. Provisional Patent Application Ser. No. 63/288,788, filed on Dec. 13, 2021, the disclosure of which is hereby incorporated by reference. The application is incorporated herein by reference in their entirety.

### STATEMENT REGARDING FEDERALLY SPONSORED RESEARCH

This invention was made with government support under GM122567 awarded by National Institutes of Health. The government has certain rights in the invention.

### FIELD OF THE INVENTION

This disclosure relates generally to fusion constructs optically controllable by far red light and methods of use thereof.

### BACKGROUND OF THE INVENTION

Receptor tyrosine kinases (RTKs) are single-pass transmembrane receptors regulated by growth factors and hormones and involved in cell proliferation, migration, metabolism, and differentiation. Similar to other single-pass receptors, RTKs consist of an extracellular (ligand-binding) domain, a transmembrane domain, and a cytoplasmic domain that, in turn, is composed of the juxtamembrane domain and catalytic domain.

Activation of RTKs with their ligands in replacement therapy is considered a promising option for the treatment of neurodegeneration, wound healing, and diabetes. However, non-targeted action of injected RTK ligands can lead to undesirable effects or diminishing efficiency of therapy. As opposed to activation of RTKs with diffusible ligands, regulation of RTK activity with light should allow non-invasive, spatially and temporally precise, and reversible control of downstream signaling and can be used for basic research and, in perspective, as an alternative for replacement therapy.

The light-control of RTK signaling can be achieved with optically-controlled RTKs (opto-RTKs). However, the available opto-RTKs are activated with visible light that poorly penetrates animal tissues and, therefore, requires implantation of optical fibers and tethering animals (Leopold, A. V., et al. Chem Sci 11, 10019-10034 (2020); Piatkevich, K. D., et al. Chem Soc Rev 42, 3441-3452 (2013)). For non-invasive deep-tissue light-control and detection of cell signaling, genetically encoded probes operated in far-red (FR) and near-infrared (NIR) light are required. Recently, a cyanobacterial phytochrome 1 (Cph1) from Synechocystis was used to engineer the FR light-controllable TrkB. However, the functioning of Cph1 requires a phycocyanobilin (PCB) chromophore, which is not naturally present in mammalian cells and needs to be supplied exogenously.

Therefore, there remains a strong need for a novel strategy for light-induced gene transcription control.

### SUMMARY OF THE INVENTION

This disclosure addresses the need mentioned above in a number of aspects. In one aspect, this disclosure provides a polynucleotide encoding a chimeric polypeptide. The chimeric polypeptide comprises (a) an extracellular light-responsive polypeptide, (b) a transmembrane domain linked to the C-terminus of the light-responsive polypeptide, and (c) an intracellular domain of a receptor linked to the C-terminus of the transmembrane domain, wherein the light-responsive polypeptide, when associated with a chromophore, is capable of switching from a first state to a second state when exposed to illumination by a wavelength, and wherein the intracellular domain of the receptor is activated at the second state.

In some embodiments, the intracellular domain of the receptor dimerizes at the second state. In some embodiments, the intracellular domain of the receptor exists as an inactive dimer at the first state and exists as an active dimer at the second state.

In some embodiments, the light-responsive polypeptide comprises an N-terminal photosensory core module (PCM) of Deinococcus radiodurance bacteriophytochrome (DrBphP-PCM) or a variant thereof.

In some embodiments, the light-responsive polypeptide comprises an amino acid sequence having at least 90% sequence identity to the amino acid sequence of any one of SEQ ID NOs: 2-3 or comprises the amino acid sequence of any one of SEQ ID NOs: 2-3.

In some embodiments, the receptor is a receptor tyrosine kinase (RTK). In some embodiments, the receptor tyrosine kinase is selected from EGFR, HER2, FGFR1, TrkA, TrkB, cKIT, cMet, and Insulin receptor (IR1).

In some embodiments, the transmembrane domain comprises a transmembrane domain of the receptor tyrosine kinase or a variant thereof. In some embodiments, the transmembrane domain comprises a transmembrane domain of EGFR, HER2, or a variant thereof. In some embodiments, the transmembrane domain further comprises one or more repeats of Tyrosine (Y)-Phenylalanine (F).

In some embodiments, the transmembrane domain comprises an amino acid sequence having at least 90% sequence identity to the amino acid sequence of any one of SEQ ID NOs: 4-7 or comprises the amino acid sequence of any one of SEQ ID NOs: 4-7.

In some embodiments, the intracellular domain is a tyrosine kinase domain of a second receptor tyrosine kinase. In some embodiments, the second receptor tyrosine kinase is selected from EGFR, HER2, FGFR1, TrkA, TrkB, cKIT, cMet, and IR1.

In some embodiments, the intracellular domain comprises an amino acid sequence having at least 90% sequence identity to the amino acid sequence of any one of SEQ ID NOs: 8-15 or comprises the amino acid sequence of any one of SEQ ID NOs: 8-15.

In some embodiments, the chimeric polypeptide further comprises a signaling peptide linked to the N-terminus of the light-responsive polypeptide. In some embodiments, the signaling peptide comprises an Igκ signaling peptide.

In some embodiments, the signaling peptide comprises an amino acid sequence having at least 90% sequence identity to the amino acid sequence of SEQ ID NO: 16 or comprises the amino acid sequence of SEQ ID NO: 16

In some embodiments, the chimeric polypeptide further comprises a Golgi-export peptide linked to the C-terminus of the intracellular domain. In some embodiments, the Golgi-export peptide comprises an amino acid sequence having at least 90% sequence identity to the amino acid sequence of any one of SEQ ID NOs: 17-19 or comprises the amino acid sequence of any one of SEQ ID NOs: 17-19.

In some embodiments, the light-responsive polypeptide is linked to the transmembrane domain via a peptide linker. In some embodiments, the transmembrane domain is linked to the intracellular domain via a peptide linker.

In some embodiments, the chimeric polypeptide comprises an amino acid sequence having at least 90% sequence identity to the amino acid sequence of any one of SEQ ID NOs: 20-27 or comprises the amino acid sequence of any one of SEQ ID NOs: 20-27.

In some embodiments, the wavelength is in far-red or near-infrared spectrum. In some embodiments, the wavelength is from about 650 nm to about 900 nm. In some embodiments, the wavelength is from about 650 nm to about 700 nm. In some embodiments, the wavelength is from about 700 nm to about 780 nm.

In another aspect, this disclosure provides a polypeptide encoded by a polynucleotide disclosed herein, a vector comprising the polynucleotide, and a cell comprising the polynucleotide or the vector, as disclosed herein.

In another aspect, this disclosure also provides a composition comprising a polynucleotide, a polypeptide, a vector, or a cell, as disclosed herein. In some embodiments, the vector comprises a viral vector. In some embodiments, the viral vector comprises an adeno-associated viral vector, lentiviral vector or adenoviral vector. In some embodiments, the adeno-associated viral vector is selected from AAV1, AAV2, AAV3, AAV4, AAV5, AAV6, AAV7, AAV8, AAV9, AAV10, AAV11, AAV 12, AAV13, AAV rh74, and recombinant subtypes thereof.

Also within the scope of this disclosure is a kit comprising a polynucleotide, a polypeptide, a vector, a cell, or a composition, as disclosed herein.

In another aspect, this disclosure further provides a method for modulating (e.g., inhibiting, activating) an expression level of a gene in a cell. The method comprises: (a) introducing to the cell a polynucleotide or a vector, as disclosed herein; and exposing the cell to illumination by an activation wavelength to modulate the expression level of the gene; or (b) providing a polypeptide or a cell, as disclosed herein; and exposing the polypeptide or the cell to illumination by the activation wavelength to modulate the expression level of the gene.

In another aspect, this disclosure also provides a method for modulating (e.g., inhibiting, activating) an expression level of a gene in a subject. The method comprises: introducing to the subject a polynucleotide or a vector, as disclosed herein; and exposing the subject to illumination by an activation wavelength to modulate the expression level of the gene in the subject. In some embodiments, the subject is exposed to illumination at a site of the subject where modulation of the expression level of the gene is needed.

In some embodiments, the gene is regulated by a receptor tyrosine kinase.

In some embodiments, the activation wavelength is in far-red or near-infrared spectrum. In some embodiments, the wavelength is from about 650 nm to about 900 nm. In some embodiments, the wavelength is from about 650 nm to about 700 nm. In some embodiments, the wavelength is from about 700 nm to about 780 nm.

In another aspect, this disclosure additionally provides a method for identifying a modulator capable of modulating (e.g., inhibiting, activating) an activity or expression level of a receptor. The method comprises: (a) contacting the modulator with a cell disclosed herein; (b) illuminating the cell by a wavelength; (c) measuring the activity or expression level of the receptor in the cell and in a control cell that has not been contacted with the modulator; (d) comparing the activity or expression level of the receptor in the cell to the activity or expression level of the receptor in the control cell; and (e) identifying the modulator as having modulating activity for the receptor if a difference between the activity or expression level of the receptor in the cell and the activity or expression level of the receptor in the control cell is greater or less than a reference value.

In some embodiments, the receptor is a receptor tyrosine kinase. In some embodiments, the receptor tyrosine kinase is selected from EGFR, HER2, FGFR1, TrkA, TrkB, cKIT, cMet, and IR1. In some embodiments, the modulator is an inhibitor or activator.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1a, 1b, 1c, and 1d are a set of diagrams showing structural predeterminants of opto-RTK engineering. FIG. 1a shows existing hypotheses of RTK activation. Top: RTK dimerization: inactive RTKs exist as monomers and ligand binding causes their dimerization and consequent activation. Bottom: Rotational coupling: inactive RTKs exist as preformed dimers and ligand binding causes conformational changes resulting in RTK activation. FIG. 1b shows structural changes occurring in EGFR receptor. Left: EGFR receptor exists as a preformed inactive dimer. Right: EGF ligand binding causes conformational changes and EGFR activation. FIG. 1c shows light-induced conformational changes in DrBphP-PCM obligate dimer causing distance increase between C-termini of DrBphP-PCM protomers (Takala, H., et al. Biochemistry 53, 7076-7085 (2014)). FIG. 1d shows a schematic representation of the opto-RTK design (top) and mechanism of its activation (bottom). DrBphP-PCM targeted to the extracellular surface by Igκ signaling peptide (sp) is connected to cytoplasmic RTK domain (cytoRTK) via transmembrane (tmRTK) domain. In darkness or NIR light, opto-RTK remains inactive. FR light causes DrBphP-PCM conformational changes, which are transmitted to cytoplasmic RTK domains, causing their re-orientation and trans-phosphorylation.

FIGS. 2a, 2b, 2c, and 2d are a set of diagrams showing engineering and characterization of opto-EGFR and opto-HER2 prototypes. FIG. 2a (left) shows ligand binding causes RTK autophosphorylation, interaction with Grb2 and SOS, and results in activation of ERK1/2 pathway consisting of RAS, RAF, MEK, and ERK1/2 kinases. Consequently, ERK1/2 activation leads to immediate early gene (IEG) expression driven by transcription factor Elk-1. FIG. 2a (right) shows activation of chimeric opto-RTK with far-red light leads to activation of ERK1/2 pathway and induction of Elk-1 dependent IEG expression. FIG. 2b shows a scheme of luciferase reporter assay. Top: ERK1/2 is inactive, and Elk-1 fused to Gal4DBD is monomeric and inactive. Bottom: ERK1/2 is active, and activated ERK1/2 phosphorylates Elk-1. Phosphorylated Elk-1-Gal4DBD fusion dimerizes, binds to 5×UAS sequence, and drives luciferase reporter expression. FIGS. 2c and 2d show light-induced activation of Elk-1-dependent luciferase expression by opto-EGFR (FIG. 2c) and opto-HER2 (FIG. 2d) prototypes in PC6-3 cells. In the darkness, luciferase expression is suppressed. 660 nm FR light activates eDrRTKs and upregulates luciferase expression. Luciferase was detected after 24 h of illumination. 25 μM BV was added to the culture medium in all experiments. Error bars represent s.d., n=3 experiments.

FIG. 3 shows an example workflow of eDrRTK design, comprising the processes as set forth below. (1) Extracellular domain of EGFR was changed to the DrBphP-PCM from the full-length bacterial DrBphP, consisting of coiled-coiled (cc) linker and histidine kinase (HisK) domain. (2) Several N-terminal secretory signals (from Igκ, EGFR and secrecon) were compared, and (3) Igκ signaling peptide was selected. (4) Different C-terminal ER-export signals were attached to the relevant constructs. (5) Several Golgi export signals were combined with the C-terminal ANSFCYENEVAL ER export signal and, finally, the Golgi export signal of Kir2.1+ channel was chosen. (6) Final eDrEGFR construct was selected, and the EGFR protein parts were swapped with the corresponding HER2, FGFR1, TrkA, TrkB, cMet, IR1, and cKIT parts. (7) Transmembrane domains (tm) of all RTKs, except for HER2, were swapped with the transmembrane domain of HER2 or EGFR. (8) Performance of FGFR1, TrkA, cMet, cKIT fusions was improved by adding to N-terminus of HER2 transmembrane domain -YF- amino acid repeats. The final eDrRTK constructs are shown in the right column. The sequences shown are YFSIVSAWGILLVWLGWFGILI (SEQ ID NO: 63), YFYFSIVSAWGILLVWLGWFGILI (SEQ ID NO: 64), and YFYFYFSIVSAWGILLVWLGWFGILI (SEQ ID NO: 65).

FIGS. 4a, 4b, 4c, 4d, 4e, 4f, 4g, and 4f are a set of diagrams showing induction of Elk-1-dependent luciferase reporter expression by eDrRTKs: eDrEGFR (FIG. 4a), eDrHER2 (FIG. 4b), eDrTrkB (FIG. 4c), eDrIR1 (FIG. 4d), eDrTrkA (FIG. 4e), eDrMet (FIG. 4f), eDrFGFR1 (FIG. 4g), and eDrcKIT (FIG. 4h). In darkness, Elk-1-dependent luciferase expression is suppressed. 660 nm FR light activates eDrRTKs and upregulates luciferase expression. Luciferase expression was detected after 24 h FR illumination. 25 μM BV was added to culture medium in all experiments. Error bars represent s.d., n=3 experiments.

FIGS. 5a, 5b, 5c, 5d, 5e, 5f, 5g, and 5f are a set of diagrams showing phosphorylation of eDrRTKs and ERK1/2 upon short-term action of far-red light. Western blots of phosphorylated and total eDrRTKs and ERK1/2 in lysates of HEK293 cells transiently transfected with relevant constructs: eDrEGFR (FIG. 5a), eDrHER2 (FIG. 5b), eDrIR1 (FIG. 5c), eDrTrkA (FIG. 5d), eDrTrkB (FIG. 5e), and eDrFGFR1 (FIG. 5f). Lane intensities (LIs) of Western blots are normalized to the corresponding GAPDH LIs. HEK293 cells were grown in darkness and 24 h after transfection were activated for 0 (black columns), 1 or 10 min with FR 660 nm light. Quantification of LIs of mock-transfected control cells is shown in grey. 25 μM BV was added to the culture medium in all experiments. Error bars represent s.d., n=3 experiments.

FIGS. 6a, 6b, and 6c are a set of diagrams showing regulation of PLCγ signaling by eDrRTKs. FIG. 6a (top) shows that eDrRTK activates PLCγ. PLCγ catalyzes PIP2 hydrolysis and formation of IP3 and DAG. IP3 interacts with IP3R channels in ER after which they become permeable to Ca²⁺. In turn, Ca²⁺ interacts with ORAI channels in the plasma membrane and induces Ca²⁺ entry from the extracellular space. Bottom: Scheme of the plasmid encoding eDrRTK and GCaMP6m Ca²⁺ indicator via IRES2. FIG. 6b (bottom) shows the results of Western blots of phosphor-PLCγ, total PLCγ, and GAPDH in HEK293 cell lysates and quantification of lane intensities (LIs) of phospho-PLCγ normalized to GAPDH LIs. FIG. 6c shows representative HEK293 cells co-transfected with eDrRTKs and GCaMP6m imaged before, during, and after 25 s illumination with 660 nm FR light. The arrows indicate the start of illumination. 25 μM BV was added to the culture medium in all experiments. Scale bars, 10 μm. Error bars represent s.d., n=5 cells.

FIGS. 7a, 7b, 7c, 7d, 7e, 7f, 7g, and 7f. Activation of eDrTrkB in neuronal cells. FIG. 7a shows that mCherry-eDrTrkB activates ERK1/2 signaling, which leads to the expression of cFos. FIG. 7b shows immunostaining of ERK1/2 translocation to the nucleus in primary rat neurons upon FR illumination. Neurons were fixed and stained for ERK1/2 (Alexa488) and chromatin (Hoechst). FIG. 7c shows quantification of ERK1/2 intensity in nucleus normalized to mCherry-eDrTrkB expression in the same neuron in darkness and upon FR illumination. FIGS. 7d and 7e show phosphorylation of eDrTrkB (FIG. 7d) and downstream ERK1/2 (FIG. 7c) in neuroblastoma N2a cells upon FR illumination analyzed by Western blot of cell lysate. The experiment and its analysis were performed as in FIG. 5. FIG. 7f shows induction of cFos expression in neuroblastoma N2a cells analyzed by Western blot of cell lysate after 0, 1, 12, and 24 h of FR illumination. 5 μM BV was added to the culture medium in all experiments. Error bars represent s.d., n=3 experiments.

FIGS. 8a, 8b, and 8c are a set of diagrams showing amounts of NREM sleep increased in mice expressing eDrTrkB in cerebral cortex following exposure to FR light. FIG. 8a is a schematic diagram depicting the AAV9 injection site and placement of the LED, EEG electrodes, and EMG wires in mice. AAV9s encoding mCherry-eDrTrkB and shMBVR-HO1-Fd-Fnr were unilaterally injected into the somatosensory cortex. EEG screw electrodes implanted above the frontal cortex and EMG electrodes in the nuchal muscles were used to assess behavioral state. An LED light source (FR: 629 nm or NIR: 810 nm) was fixed over the parietal cortex and was used for light stimulation. FIG. 9b is a diagram illustrating the optogenetic stimulation protocol. The light stimulation was applied to illuminate the somatosensory cortex for 30 s followed by 180 s of no stimulation, and this cycle was repeated for 24 h beginning at ZT8 while the sleep-wake recording continued. FIG. 8c shows unilateral optogenetic stimulation of AAV-transfected cortical cells expressing mCherry-eDrTrkB differentially affected sleep-wake behavior. The comparisons were made between the data obtained during the 24 h period of light stimulation and the preceding 24 h baseline period (blue line). LED light at 629 nm but not at 810 nm caused a decrease in the overall amount of wake and an increase in NREM sleep during the first 6 h (ZT8-14) compared to the baseline. REM sleep was not significantly affected. Mean±SEM for all the mice in each condition are given. Experiments included 629 nm light stimulation (N=7) and 810 nm light stimulation (baseline, N=5).

### DETAILED DESCRIPTION OF THE INVENTION

This disclosure provides a generalized approach for engineering receptor tyrosine kinases (RTKs) optically controlled with far-red light, named eDrRTKs, by targeting a bacterial phytochrome (e.g., DrBphP) to the cell surface and allowing its light-induced conformational changes to be transmitted across the plasma membrane via transmembrane helices to intracellular RTK domains. The ability to activate eDrRTKs with far-red light enabled cross-talk free spectral multiplexing with fluorescent probes operating in a shorter spectral range, allowing for all-optical assays, including non-invasive stimulation in the brain of a live animal. The disclosed engineering approach can be applied beyond RTKs to any membrane receptors (e.g., immune receptors, such as Toll-like receptors, Interleukin receptors, CD3ζ part of TCR receptor), channels, surface antigens, or membrane antibodies that share high similarity with RTKs in mechanisms of their activation.

### A. Fusion Constructs Optically Controllable by Far Red Light

a. Polynucleotides

In one aspect, this disclosure provides a polynucleotide encoding a chimeric polypeptide. The chimeric polypeptide comprises (a) an extracellular light-responsive polypeptide, (b) a transmembrane domain linked to the C-terminus of the light-responsive polypeptide, and (c) an intracellular domain (e.g., cytoplasmic domain) of a receptor linked to the C-terminus of the transmembrane domain, wherein the light-responsive polypeptide, when associated with a chromophore, is capable of switching from a first state to a second state when exposed to illumination by a wavelength, and wherein the intracellular domain of the receptor is activated at the second state.

In some embodiments, the intracellular domain of the receptor dimerizes at the second state. In some embodiments, the intracellular domain of the receptor exists as an inactive dimer at the first state and exists as an active dimer at the second state.

In some embodiments, the light-responsive polypeptide comprises an N-terminal photosensory core module (PCM) of Deinococcus radiodurance bacteriophytochrome (DrBphP-PCM) or a variant thereof.

In some embodiments, the light-responsive polypeptide comprises an amino acid sequence having at least 80% (e.g., 80%, 85%, 90%, 91%, 92%, 93%, 94%, 95%, 96%, 97%, 98%, 99%) sequence identity to the amino acid sequence of any one of SEQ ID NOs: 2-3 or comprises the amino acid sequence of any one of SEQ ID NOs: 2-3 (see Table 1).

The terms “light-responsive” and “light-activated” are used herein interchangeably. The terms “light-responsive polypeptide,” “light-responsive protein,” “light-activated protein,” and “light-activated protein” mean a polypeptide or protein that undergoes a conformational change when exposed to light of an activating wavelength.

As used herein, the term “chimeric protein” or “chimeric polypeptide” refers to a recombinant fusion protein, e.g., a single polypeptide having the extracellular domains described herein and, optionally, a linker. For example, in some embodiments, the chimeric protein is translated as a single peptide chain in a cell. In some embodiments, a chimeric protein refers to a recombinant protein of multiple polypeptides, e.g., multiple domains described herein, that are linked to yield a single unit, e.g., in vitro (e.g., with one or more synthetic linkers described herein).

As used herein, the term “extracellular” refers to the protein portion extended from cell surface. “Extracellular domain,” as used herein, refers broadly to the portion of a protein that extends from the surface of a cell. In some embodiments, an extracellular domain refers to a portion of a transmembrane protein that is capable of interacting with the extracellular environment. In some embodiments, an extracellular domain refers to a portion of a transmembrane protein that is sufficient to bind to a ligand or receptor and effectively transmit a signal to a cell. In some embodiments, an extracellular domain is the entire amino acid sequence of a transmembrane protein which is external of a cell or the cell membrane. In some embodiments, an extracellular domain is the portion of an amino acid sequence of a transmembrane protein that is external of a cell or the cell membrane and is needed for signal transduction and/or ligand binding as may be assayed using methods known in the art (e.g., in vitro ligand binding and/or cellular activation assays).

“Transmembrane domain,” as used herein, refers broadly to an amino acid sequence (e.g., with about 15 to 50 amino acid residues in length) which spans the plasma membrane. In some embodiments, a transmembrane domain includes about at least 20, 25, 30, 35, 40, or 45 amino acid residues and spans the plasma membrane. Transmembrane domains are rich in hydrophobic residues, and typically have an alpha-helical structure. In an embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta, et al. (1996) Annu. Rev. Neurosci. 19:235-263.

As used herein, the term “intracellular domain,” “intracellular signaling domain,” or “cytoplasmic domain” refers to the intracellular portion of a molecule. In some embodiments, an intracellular domain transmits a signal to an effector function and causes a cell to perform a specific function, e.g., activation, phosphorylation, cytokine production, etc. The term intracellular domain is meant to include any truncated portion of the intracellular domain sufficient to transduce an effector function signal.

The terms “chromophore,” “photoactivating agent,” and “photoactivator” are used herein interchangeably. A chromophore means a chemical compound which, when contacted by light irradiation, is capable of absorbing the light. The chromophore readily undergoes photoexcitation and can then transfer its energy to other molecules or emit it as light. Phytochromes are photosensory receptors found in plants, fungi, bacteria and cyanobacteria that absorb light in the red and far-red part of spectrum and utilize linear tetrapyrrole bilins, such as biliverdin IXa (BV), phycocyanobilin or phytochromobilin, as chromophores. Bacterial phytochromes, also termed bacteriophytochrome photoreceptors (BphPs), i.e., D. radiodurance bacteriophytochrome, use BV as a chromophore.

TABLE 1
Representative Sequences

| SEQ | | |
|---|---|---|
| ID | | OTHER |
| NO | SEQUENCES | INFORMATION |
| 1 | MSRDPLPFFPPLYLGGPEITTENCE | DrBphP |
| | REPIHIPGSIQPHGALLTADGHSGE | |
| | VLQMSLNAATFLGQEPTVLRGQTLA | |
| | ALLPEQWPALQAALPPGCPDALQYR | |
| | ATLDWPAAGHLSLTVHRVGELLILE | |
| | FEPTEAWDSTGPHALRNAMFALESA | |
| | PNLRALAEVATQTVRELTGFDRVML | |
| | YKFAPDATGEVIAEARREGLHAFLG | |
| | HRFPASDIPAQARALYTRHLLRLTA | |
| | DTRAAAVPLDPVLNPQTNAPTPLGG | |
| | AVLRATSPMHMQYLRNMGVGSSLSV | |
| | SVVVGGQLWGLIACHHQTPYVLPPD | |
| | LRTTLEYLGRLLSLQVQVKEAADVA | |
| | AFRQSLREHHARVALAAAHSLSPHD | |
| | TLSDPALDLLGLMRAGGLILRFEGR | |
| | WQTLGEVPPAPAVDALLAWLETQPG | |
| | ALVQTDALGQLWPAGADLAPSAAGL | |
| | LAISVGEGWSECLVWLRPELRLEVA | |
| | WGGATPDQAKDDLGPRHSFDTYLEE | |
| | KRGYAEPWHPGEIEEAQDLRDTLTG | |
| | ALGERLSVIRDLNRALTQSNAEWRQ | |
| | YGFVISHHMQEPVRLISQFAELLTR | |
| | QPRAQDGSPDSPQTERITGFLLRET | |
| | SRLRSLTQDLHTYTALLSAPPPVRR | |
| | PTPLGRVVDDVLQDLEPRIADTGAS | |
| | IEVAPELPVIAADAGLLRDLLLHLI | |
| | GNALTFGGPEPRIAVRTERQGAGWS | |
| | IAVSDQGAGIAPEYQERIFLLFQRL | |
| | GSLDEALGNGLGLPLCRKIAELHGG | |
| | TLTVESAPGEGSTFRCWLPDAGPLP | |
| | GAADA | |
| 2 | MSRDPLPFFPPLYLGGPEITTENCE | DrBphP-PCM |
| | REPIHIPGSIQPHGALLTADGHSGE | |
| | VLQMSLNAATFLGQEPTVLRGQTLA | |
| | ALLPEQWPALQAALPPGCPDALQYR | |
| | ATLDWPAAGHLSLTVHRVGELLILE | |
| | FEPTEAWDSTGPHALRNAMFAFESA | |
| | PNLRALAEVATQTVRELTGFDRVML | |
| | YKFAPDATGEVIAEARREGLHAFLG | |
| | HRFPASDIPAQARALYTRHLLRLTA | |
| | DTRAATVPLDPVLNPQTNAPTPLGG | |
| | AVLRATSPMHMQYLRNMGVGSSLSV | |
| | SVVVGGQLWGLIACHHQTPYVLPPD | |
| | LRTTLEYLGRLLSLQVQVKEAADVA | |
| | AFRQSLREHHARVALAAAHSLSPHD | |
| | TLSDPALDLLGLMRAGGLILRFEGR | |
| | WQTLGEVPPAPAVDALLAWLETQPG | |
| | ALVQTDALGQLWLAGADLAPSAAGL | |
| | LAISVGEGWSECLVWLRPELRLEVA | |
| | WGGATPDQAKDDLGPRHSFDTYLEE | |
| | KRGYAEPWHPGEIEEAQDLRDTLTG | |
| | ALGE | |
| 3 | KGEEDNMAIIKEFMRFKVHMEGSVN | DrBphP-PCM |
| | GHEFEIEGEGEGRPYEGTQTAKLKV | with N- |
| | TKGGPLPFAWDILSPQFMYGSKAYV | terminal |
| | KHPADIPDYLKLSFPEGFKWERVMN | mCherry |
| | FEDGGVVTVTQDSSLQDGEFIYKVK | fluorescent |
| | LRGTNFPSDGPVMQKKTMGWEASSE | protein |
| | RMYPEDGALKGEIKQRLKLKDGGHY | |
| | DAEVKTTYKAKKPVQLPGAYNVNIK | |
| | LDITSHNEDYTIVEQYERAEGRHST | |
| | GGMDELYKEFSAGSAGSAGTGMSRD | |
| | PLPFFPPLYLGGPEITTENCEREPI | |
| | HIPGSIQPHGALLTADGHSGEVLQM | |
| | SLNAATFLGQEPTVLRGQTLAALLP | |
| | EQWPALQAALPPGCPDALQYRATLD | |
| | WPAAGHLSLTVHRVGELLILEFEPT | |
| | EAWDSTGPHALRNAMFAFESAPNLR | |
| | ALAEVATQTVRELTGFDRVMLYKFA | |
| | PDATGEVIAEARREGLHAFLGHRFP | |
| | ASDIPAQARALYTRHLLRLTADTRA | |
| | ATVPLDPVLNPQTNAPTPLGGAVLR | |
| | ATSPMHMQYLRNMGVGSSLSVSVVV | |
| | GGQLWGLIACHHQTPYVLPPDLRTT | |
| | LEYLGRLLSLQVQVKEAADVAAFRQ | |
| | SLREHHARVALAAAHSLSPHDTLSD | |
| | PALDLLGLMRAGGLILRFEGRWQTL | |
| | GEVPPAPAVDALLAWLETQPGALVQ | |
| | TDALGQLWLAGADLAPSAAGLLAIS | |
| | VGEGWSECLVWLRPELRLEVAWGGA | |
| | TPDQAKDDLGPRHSFDTYLEEKRGY | |
| | AEPWHPGEIEEAQDLRDTLTGALGE | |
| 4 | IATGMVGALLLLLVVALGIGLFM | Transmembrane |
| | | domain |
| | | (EGFR) |
| 5 | SIVSAVVGILLVVVLGVVFG | Transmembrane |
| | | domain |
| | | (HER2) |
| 6 | YFYFIATGMVGALLLLLVVALGIGL | Transmembrane |
| | FM | domain |
| | | (EGFR) with |
| | | YF repeats |
| 7 | YFYFSIVSAVVGILLVVVLGVVFG | Transmembrane |
| | | domain |
| | | (HER2) with |
| | | YF repeats |
| 8 | RRRHIVRKRTLRRLLQERELVEPLT | Intracellular |
| | PSGEAPNQALLRILKETEFKKIKVL | domain |
| | GSGAFGTVYKGLWIPEGEKVKIPVA | (EGFR) |
| | IKELREATSPKANKEILDEAYVMAS | |
| | VDNPHVCRLLGICLTSTVQLITQLM | |
| | PFGCLLDYVREHKDNIGSQYLLNWC | |
| | VQIAKGMNYLEDRRLVHRDLAARNV | |
| | LVKTPQHVKITDFGLAKLLGAEEKE | |
| | YHAEGGKVPIKWMALESILHRIYTH | |
| | QSDVWSYGVTVWELMTFGSKPYDGI | |
| | PASEISSILEKGERLPQPPICTIDV | |
| | YMIMVKCWMIDADSRPKFRELIIEF | |
| | SKMARDPQRYLVIQGDERMHLPSPT | |
| | DSNFYRALMDEEDMDDVVDADEYLI | |
| | PQQGFFSSPSTSRTPLLSSLSATSN | |
| | NSTVACIDRNGLQSCPIKEDSFLQR | |
| | YSSDPTGALTEDSIDDTFLPVPEYI | |
| | NQSVPKRPAGSVQNPVYHNQPLNPA | |
| | PSRDPHYQDPHSTAVGNPEYLNTVQ | |
| | PTCVNSTFDSPAHWAQKGSHQISLD | |
| | NPDYQQDFFPKEAKPNGIFKGSTAE | |
| | NAEYLRVAPQSSEFIGA | |
| 9 | SIISAVVGILLVVVLGVVFGILIKR | Intracellular |
| | RQQKIRKYTMRRLLQETELVEPLTP | domain |
| | SGAMPNQAQMRILKETELRKVKVLG | (HER2) |
| | SGAFGTVYKGIWIPDGENVKIPVAI | |
| | KVLRENTSPKANKEILDEAYVMAGV | |
| | GSPYVSRLLGICLTSTVQLVTQLMP | |
| | YGCLLDHVRENRGRLGSQDLLNWCM | |
| | QIAKGMSYLEDVRLVHRDLAARNVL | |
| | VKSPNHVKITDFGLARLLDIDETEY | |
| | HADGGKVPIKWMALESILRRRFTHQ | |
| | SDVWSYGVTVWELMTFGAKPYDGIP | |
| | AREIPDLLEKGERLPQPPICTIDVY | |
| | MIMVKCWMIDSECRPRFRELVSEFS | |
| | RMARDPQRFVVIQNEDLGPASPLDS | |
| | TFYRSLLEDDDMGDLVDAEEYLVPQ | |
| | QGFFCPDPAPGAGGMVHHRHRSSST | |
| | RSGGGDLTLGLEPSEEEAPRSPLAP | |
| | SEGAGSDVFDGDLGMGAAKGLQSLP | |
| | THDPSPLQRYSEDPTVPLPSETDGY | |
| | VAPLTCSPQPEYVNQPDVRPQPPSP | |
| | REGPLPAARPAGATLERPKTLSPGK | |
| | NGVVKDVFAFGGAVENPEYLTPQGG | |
| | AAPQPHPPPAFSPAFDNLYYWDQDP | |
| | PERGAPPSTFKGTPTAENPEYLGLD | |
| | VPV | |
| 10 | KMKSGTKKSDFHSQMAVHKLAKSIP | Intracellular |
| | LRRQVTVSADSSASMNSGVLLVRPS | domain |
| | RLSSSGTPMLAGVSEYELPEDPRWE | (FGFR1) |
| | LPRDRLVLGKPLGEGCFGQVVLAEA | |
| | IGLDKDKPNRVTKVAVKMLKSDATE | |
| | KDLSDLISEMEMMKMIGKHKNIINL | |
| | LGACTQDGPLYVIVEYASKGNLREY | |
| | LQARRPPGLEYCYNPSHNPEEQLSS | |
| | KDLVSCAYQVARGMEYLASKKCIHR | |
| | DLAARNVLVTEDNVMKIADFGLARD | |
| | IHHIDYYKKTTNGRLPVKWMAPEAL | |
| | FDRIYTHQSDVWSFGVLLWEIFTLG | |
| | GSPYPGVPVEELFKLLKEGHRMDKP | |
| | SNCTNELYMMMRDCWHAVPSQRPTF | |
| | KQLVEDLDRIVALTSNQEYLDLSMP | |
| | LDQYSPSFPDTRSSTCSSGEDSVFS | |
| | HEPLPEEPCLPRHPAQLANGGLKRR | |
| 11 | NKCGRRNKFGINRPAVLAPEDGLAM | Intracellular |
| | SLHFMTLGGSSLSPTEGKGSGLQGH | domain (TrkA) |
| | IIENPQYFSDACVHHIKRRDIVLKW | |
| | ELGEGAFGKVFLAECHNLLPEQDKM | |
| | LVAVKALKEASESARQDFQREAELL | |
| | TMLQHQHIVRFFGVCTEGRPLLMVF | |
| | EYMRHGDLNRFLRSHGPDAKLLAGG | |
| | EDVAPGPLGLGQLLAVASQVAAGMV | |
| | YLAGLHFVHRDLATRNCLVGQGLVV | |
| | KIGDFGMSRDIYSTDYYRVGGRTML | |
| | PIRWMPPESILYRKFTTESDVWSFG | |
| | VVLWEIFTYGKQPWYQLSNTEAIDC | |
| | ITQGRELERPRACPPEVYAIMRGCW | |
| | QREPQQRHSIKDVHARLQALAQAPP | |
| | VYLDVLG | |
| 12 | KLARHSKFGMKGPASVISNDDDSAS | Intracellular |
| | PLHHISNGSNTPSSSEGGPDAVIIG | domain (TrkB) |
| | MTKIPVIENPQYFGITNSQLKPDTF | |
| | VQHIKRHNIVLKRELGEGAFGKVFL | |
| | AECYNLCPEQDKILVAVKTLKDASD | |
| | NARKDFHREAELLTNLQHEHIVKFY | |
| | GVCVEGDPLIMVFEYMKHGDLNKFL | |
| | RAHGPDAVLMAEGNPPTELTQSQML | |
| | HIAQQIAAGMVYLASQHFVHRDLAT | |
| | RNCLVGENLLVKIGDFGMSRDVYST | |
| | DYYRVGGHTMLPIRWMPPESIMYRK | |
| | FTTESDVWSLGVVLWEIFTYGKQPW | |
| | YQLSNNEVIECITQGRVLQRPRTCP | |
| | QEVYELMLGCWQREPHMRKNIKGIH | |
| | TLLQNLAKASPVYLDILG | |
| 13 | KYLQKPMYEVQWKVVEEINGNNYVY | Intracellular |
| | IDPTQLPYDHKWEFPRNRLSFGKTL | domain (cKIT) |
| | GAGAFGKVVEATAYGLIKSDAAMTV | |
| | AVKMLKPSAHLTEREALMSELKVLS | |
| | YLGNHMNIVNLLGACTIGGPTLVIT | |
| | EYCCYGDLLNFLRRKRDSFICSKQE | |
| | DHAEAALYKNLLHSKESSCSDSTNE | |
| | YMDMKPGVSYVVPTKADKRRSVRIG | |
| | SYIERDVTPAIMEDDELALDLEDLL | |
| | SFSYQVAKGMAFLASKNCIHRDLAA | |
| | RNILLTHGRITKICDFGLARDIKND | |
| | SNYVVKGNARLPVKWMAPESIFNCV | |
| | YTFESDVWSYGIFLWELFSLGSSPY | |
| | PGMPVDSKFYKMIKEGFRMLSPEHA | |
| | PAEMYDIMKTCWDADPLKRPTFKQI | |
| | VQLIEKQISESTNHIYSNLANCSPN | |
| | RQKPVVDHSVRINSVGSTASSSQPL | |
| | LVHDDV | |
| 14 | KKRKQIKDLGSELVRYDARVHTPHL | Intracellular |
| | DRLVSARSVSPTTEMVSNESVDYRA | domain (cMet) |
| | TFPEDQFPNSSQNGSCRQVQYPLTD | |
| | MSPILTSGDSDISSPLLQNTVHIDL | |
| | SALNPELVQAVQHVVIGPSSLIVHE | |
| | NEVIGRGHFGCVYHGTLLDNDGKKI | |
| | HCAVKSLNRITDIGEVSQFLTEGII | |
| | MKDFSHPNVLSLLGICLRSEGSPLV | |
| | VLPYMKHGDLRNFIRNETHNPTVKD | |
| | LIGFGLQVAKGMKYLASKKFVHRDL | |
| | AARNCMLDEKFTVKVADFGLARDMY | |
| | DKEYYSVHNKTGAKLPVKWMALESL | |
| | QTQKFTTKSDVWSFGVLLWELMTRG | |
| | APPYPDVNTFDITVYLLQGRRLLQP | |
| | EYCPDPLYEVMLKCWHPKAEMRPSF | |
| | SELVSRISAIFSTFIGEHYVHVNAT | |
| | YVNVKCVAPYPSLLSSEDNADDEVD | |
| | TRPASFWETS | |
| 15 | RKRQPDGPLGPLYASSNPEYLSASD | Intracellular |
| | VFPCSVYVPDEWEVSREKITLLREL | domain (IR1) |
| | GQGSFGMVYEGNARDIIKGEAETRV | |
| | AVKTVNESASLRERIEFLNEASVMK | |
| | GFTCHHVVRLLGVVSKGQPTLVVME | |
| | LMAHGDLKSYLRSLRPEAENNPGRP | |
| | PPTLQEMIQMAAEIADGMAYLNAKK | |
| | FVHRDLAARNCMVAHDFTVKIGDFG | |
| | MTRDIYETDYYRKGGKGLLPVRWMA | |
| | PESLKDGVFTTSSDMWSFGVVLWEI | |
| | TSLAEQPYQGLSNEQVLKFVMDGGY | |
| | LDQPDNCPERVTDLMRMCWQFNPKM | |
| | RPTFLEIVNLLKDDLHPSFPEVSFF | |
| | HSEENKAPESEELEMEFEDMENVPL | |
| | DRSSHCQREEAGGRDGGSSLGFKRS | |
| | YEEHIPYTHMNGGKKNGRILTLPRS | |
| | NPS | |
| 16 | METDTLLLWVLLLWVPGSTGDS | Signaling |
| | | peptide Ig κ |
| 17 | RSRFVKKD | Golgi-export |
| | | sequence |
| 18 | SYLANEIL | Golgi-export |
| | | sequence |
| 19 | FCYENEVALS | ER-export |
| | | sequence |
| 20 | METDTLLLWVLLLWVPGSTGDSKGE | Fusion |
| | EDNMAIIKEFMRFKVHMEGSVNGHE | construct |
| | FEIEGEGEGRPYEGTQTAKLKVTKG | (EGFR) |
| | GPLPFAWDILSPQFMYGSKAYVKHP | |
| | ADIPDYLKLSFPEGFKWERVMNFED | |
| | GGVVTVTQDSSLQDGEFIYKVKLRG | |
| | TNFPSDGPVMQKKTMGWEASSERMY | |
| | PEDGALKGEIKQRLKLKDGGHYDAE | |
| | VKTTYKAKKPVQLPGAYNVNIKLDI | |
| | TSHNEDYTIVEQYERAEGRHSTGGM | |
| | DELYKEFSAGSAGSAGTGMSRDPLP | |
| | FFPPLYLGGPEITTENCEREPIHIP | |
| | GSIQPHGALLTADGHSGEVLQMSLN | |
| | AATFLGQEPTVLRGQTLAALLPEQW | |
| | PALQAALPPGCPDALQYRATLDWPA | |
| | AGHLSLTVHRVGELLILEFEPTEAW | |
| | DSTGPHALRNAMFAFESAPNLRALA | |
| | EVATQTVRELTGFDRVMLYKFAPDA | |
| | TGEVIAEARREGLHAFLGHRFPASD | |
| | IPAQARALYTRHLLRLTADTRAATV | |
| | PLDPVLNPQTNAPTPLGGAVLRATS | |
| | PMHMQYLRNMGVGSSLSVSVVVGGQ | |
| | LWGLIACHHQTPYVLPPDLRTTLEY | |
| | LGRLLSLQVQVKEAADVAAFRQSLR | |
| | EHHARVALAAAHSLSPHDTLSDPAL | |
| | DLLGLMRAGGLILRFEGRWQTLGEV | |
| | PPAPAVDALLAWLETQPGALVQTDA | |
| | LGQLWLAGADLAPSAAGLLAISVGE | |
| | GWSECLVWLRPELRLEVAWGGATPD | |
| | QAKDDLGPRHSFDTYLEEKRGYAEP | |
| | WHPGEIEEAQDLRDTLTGALGELEI | |
| | ATGMVGALLLLLVVALGIGLFMRRR | |
| | HIVRKRTLRRLLQERELVEPLTPSG | |
| | EAPNQALLRILKETEFKKIKVLGSG | |
| | AFGTVYKGLWIPEGEKVKIPVAIKE | |
| | LREATSPKANKEILDEAYVMASVDN | |
| | PHVCRLLGICLTSTVQLITQLMPFG | |
| | CLLDYVREHKDNIGSQYLLNWCVQI | |
| | AKGMNYLEDRRLVHRDLAARNVLVK | |
| | TPQHVKITDFGLAKLLGAEEKEYHA | |
| | EGGKVPIKWMALESILHRIYTHQSD | |
| | VWSYGVTVWELMTFGSKPYDGIPAS | |
| | EISSILEKGERLPQPPICTIDVYMI | |
| | MVKCWMIDADSRPKFRELIIEFSKM | |
| | ARDPQRYLVIQGDERMHLPSPTDSN | |
| | FYRALMDEEDMDDVVDADEYLIPQQ | |
| | GFFSSPSTSRTPLLSSLSATSNNST | |
| | VACIDRNGLQSCPIKEDSFLQRYSS | |
| | DPTGALTEDSIDDTFLPVPEYINQS | |
| | VPKRPAGSVQNPVYHNQPLNPAPSR | |
| | DPHYQDPHSTAVGNPEYLNTVQPTC | |
| | VNSTFDSPAHWAQKGSHQISLDNPD | |
| | YQQDFFPKEAKPNGIFKGSTAENAE | |
| | YLRVAPQSSEFIGASRGGGGSGGGG | |
| | SGGGGSGGGGSRSRFVKKDSAGSAG | |
| | SAGSAGSYLANEILWGSAGSAGSAG | |
| | SAGFCYENEVALS | |
| 21 | METDTLLLWVLLLWVPGSTGDSKGE | Fusion |
| | EDNMAIIKEFMRFKVHMEGSVNGHE | construct |
| | FEIEGEGEGRPYEGTQTAKLKVTKG | (HER2) |
| | GPLPFAWDILSPQFMYGSKAYVKHP | |
| | ADIPDYLKLSFPEGFKWERVMNFED | |
| | GGVVTVTQDSSLQDGEFIYKVKLRG | |
| | TNFPSDGPVMQKKTMGWEASSERMY | |
| | PEDGALKGEIKQRLKLKDGGHYDAE | |
| | VKTTYKAKKPVQLPGAYNVNIKLDI | |
| | TSHNEDYTIVEQYERAEGRHSTGGM | |
| | DELYKEFSAGSAGSAGTGMSRDPLP | |
| | FFPPLYLGGPEITTENCEREPIHIP | |
| | GSIQPHGALLTADGHSGEVLQMSLN | |
| | AATFLGQEPTVLRGQTLAALLPEQW | |
| | PALQAALPPGCPDALQYRATLDWPA | |
| | AGHLSLTVHRVGELLILEFEPTEAW | |
| | DSTGPHALRNAMFAFESAPNLRALA | |
| | EVATQTVRELTGFDRVMLYKFAPDA | |
| | TGEVIAEARREGLHAFLGHRFPASD | |
| | IPAQARALYTRHLLRLTADTRAATV | |
| | PLDPVLNPQTNAPTPLGGAVLRATS | |
| | PMHMQYLRNMGVGSSLSVSVVVGGQ | |
| | LWGLIACHHQTPYVLPPDLRTTLEY | |
| | LGRLLSLQVQVKEAADVAAFRQSLR | |
| | EHHARVALAAAHSLSPHDTLSDPAL | |
| | DLLGLMRAGGLILRFEGRWQTLGEV | |
| | PPAPAVDALLAWLETQPGALVQTDA | |
| | LGQLWLAGADLAPSAAGLLAISVGE | |
| | GWSECLVWLRPELRLEVAWGGATPD | |
| | QAKDDLGPRHSFDTYLEEKRGYAEP | |
| | WHPGEIEEAQDLRDTLTGALGELES | |
| | IISAVVGILLVVVLGVVFGILIKRR | |
| | QQKIRKYTMRRLLQETELVEPLTPS | |
| | GAMPNQAQMRILKETELRKVKVLGS | |
| | GAFGTVYKGIWIPDGENVKIPVAIK | |
| | VLRENTSPKANKEILDEAYVMAGVG | |
| | SPYVSRLLGICLTSTVQLVTQLMPY | |
| | GCLLDHVRENRGRLGSQDLLNWCMQ | |
| | IAKGMSYLEDVRLVHRDLAARNVLV | |
| | KSPNHVKITDFGLARLLDIDETEYH | |
| | ADGGKVPIKWMALESILRRRFTHQS | |
| | DVWSYGVTVWELMTFGAKPYDGIPA | |
| | REIPDLLEKGERLPQPPICTIDVYM | |
| | IMVKCWMIDSECRPRFRELVSEFSR | |
| | MARDPQRFVVIQNEDLGPASPLDST | |
| | FYRSLLEDDDMGDLVDAEEYLVPQQ | |
| | GFFCPDPAPGAGGMVHHRHRSSSTR | |
| | SGGGDLTLGLEPSEEEAPRSPLAPS | |
| | EGAGSDVFDGDLGMGAAKGLQSLPT | |
| | HDPSPLQRYSEDPTVPLPSETDGYV | |
| | APLTCSPQPEYVNQPDVRPQPPSPR | |
| | EGPLPAARPAGATLERPKTLSPGKN | |
| | GVVKDVFAFGGAVENPEYLTPQGGA | |
| | APQPHPPPAFSPAFDNLYYWDQDPP | |
| | ERGAPPSTFKGTPTAENPEYLGLDV | |
| | PVSRGGGGSGGGGSGGGGSGGGGSR | |
| | SRFVKKDSAGSAGSAGSAGSYLANE | |
| | ILWGSAGSAGSAGSAGFCYENEVAL | |
| | S | |
| 22 | METDTLLLWVLLLWVPGSTGDSKGE | Fusion |
| | EDNMAIIKEFMRFKVHMEGSVNGHE | construct |
| | FEIEGEGEGRPYEGTQTAKLKVTKG | (FGFR1) |
| | GPLPFAWDILSPQFMYGSKAYVKHP | |
| | ADIPDYLKLSFPEGFKWERVMNFED | |
| | GGVVTVTQDSSLQDGEFIYKVKLRG | |
| | TNFPSDGPVMQKKTMGWEASSERMY | |
| | PEDGALKGEIKQRLKLKDGGHYDAE | |
| | VKTTYKAKKPVQLPGAYNVNIKLDI | |
| | TSHNEDYTIVEQYERAEGRHSTGGM | |
| | DELYKEFSAGSAGSAGTGMSRDPLP | |
| | FFPPLYLGGPEITTENCEREPIHIP | |
| | GSIQPHGALLTADGHSGEVLQMSLN | |
| | AATFLGQEPTVLRGQTLAALLPEQW | |
| | PALQAALPPGCPDALQYRATLDWPA | |
| | AGHLSLTVHRVGELLILEFEPTEAW | |
| | DSTGPHALRNAMFAFESAPNLRALA | |
| | EVATQTVRELTGFDRVMLYKFAPDA | |
| | TGEVIAEARREGLHAFLGHRFPASD | |
| | IPAQARALYTRHLLRLTADTRAATV | |
| | PLDPVLNPQTNAPTPLGGAVLRATS | |
| | PMHMQYLRNMGVGSSLSVSVVVGGQ | |
| | LWGLIACHHQTPYVLPPDLRTTLEY | |
| | LGRLLSLQVQVKEAADVAAFRQSLR | |
| | EHHARVALAAAHSLSPHDTLSDPAL | |
| | DLLGLMRAGGLILRFEGRWQTLGEV | |
| | PPAPAVDALLAWLETQPGALVQTDA | |
| | LGQLWLAGADLAPSAAGLLAISVGE | |
| | GWSECLVWLRPELRLEVAWGGATPD | |
| | QAKDDLGPRHSFDTYLEEKRGYAEP | |
| | WHPGEIEEAQDLRDTLTGALGEYFY | |
| | FSIVSAVVGILLVVVLGVVFGSRKM | |
| | KSGTKKSDFHSQMAVHKLAKSIPLR | |
| | RQVTVSADSSASMNSGVLLVRPSRL | |
| | SSSGTPMLAGVSEYELPEDLRWELP | |
| | RDRLVLGKPLGEGCFGQVVLAEAIG | |
| | LDKDKPNRVTKVAVKMLKSDATEKD | |
| | LSDLISEMEMMKMIGKHKNIINLLG | |
| | ACTQDGPLYVIVEYASKGNLREYLQ | |
| | ARRPPGLEYCYNPSHNPEEQLSSKD | |
| | LVSCAYQVARGMEYLASKKCIHRDL | |
| | AARNVLVTEDNVMKIADFGLARDIH | |
| | HIDYYKKTTNGRLPVKWMAPEALFD | |
| | RIYTHQSDVWSFGVLLWEIFTLGGS | |
| | PYPGVPVEELFKLLKEGHRMDKPSN | |
| | CTNELYMMMRDCWHAVPSQRPTFKQ | |
| | LVEDLDRIVALTSNQEYLDLSMPLD | |
| | QYSPSFPDTRSSTCSSGEDSVFSHE | |
| | PLPEEPCLPRHPAQLANGGLKRRSR | |
| | GGGGSGGGGSGGGGSGGGGSRSRFV | |
| | KKDSAGSAGSAGSAGSYLANEILWG | |
| | SAGSAGSAGSAGFCYENEVALS | |
| 23 | METDTLLLWVLLLWVPGSTGDSKGE | Fusion |
| | EDNMAIIKEFMRFKHMEGSVNGHEF | construct |
| | EIEGEGEGRPYEGTQTAKLKVTKGG | (TrkA) |
| | PLPFAWDILSPQFMYGSKAYVKHPA | |
| | DIPDYLKLSFPEGFKWERVMNFEDG | |
| | GVVTVTQDSSLQDGEFIYKVKLRGT | |
| | NFPSDGPVMQKKTMGWEASSERMYP | |
| | EDGALKGEIKQRLKLKDGGHYDAEV | |
| | KTTYKAKKPVQLPGAYNVNIKLDIT | |
| | SHNEDYTIVEQYERAEGRHSTGGMD | |
| | ELYKEFSAGSAGSAGTGMSRDPLPF | |
| | FPPLYLGGPEITTENCEREPIHIPG | |
| | SIQPHGALLTADGHSGEVLQMSLNA | |
| | ATFLGQEPTVLRGQTLAALLPEQWP | |
| | ALQAALPPGCPDALQYRATLDWPAA | |
| | GHLSLTVHRVGELLILEFEPTEAWD | |
| | STGPHALRNAMFAFESAPNLRALAE | |
| | VATQTVRELTGFDRVMLYKFAPDAT | |
| | GEVIAEARREGLHAFLGHRFPASDI | |
| | PAQARALYTRHLLRLTADTRAATVP | |
| | LDPVLNPQTNAPTPLGGAVLRATSP | |
| | MHMQYLRNMGVGSSLSVSVVVGGQL | |
| | WGLIACHHQTPYVLPPDLRTTLEYL | |
| | GRLLSLQVQVKEAADVAAFRQSLRE | |
| | HHARVALAAAHSLSPHDTLSDPALD | |
| | LLGLMRAGGLILRFEGRWQTLGEVP | |
| | PAPAVDALLAWLETQPGALVQTDAL | |
| | GQLWLAGADLAPSAAGLLAISVGEG | |
| | WSECLVWLRPELRLEVAWGGATPDQ | |
| | AKDDLGPRHSFDTYLEEKRGYAEPW | |
| | HPGEIEEAQDLRDTLTGALGEYFYF | |
| | SIVSAVVGILLVVVLGVVFGSRNKC | |
| | GRRNKFGINRPAVLAPEDGLAMSLH | |
| | FMTLGGSSLSPTEGKGSGLQGHIIE | |
| | NPQYFSDACVHHIKRRDIVLKWELG | |
| | EGAFGKVFLAECHNLLPEQDKMLVA | |
| | VKALKEASESARQDFQREAELLTML | |
| | QHQHIVRFFGVCTEGRPLLMVFEYM | |
| | RHGDLNRFLRSHGPDAKLLAGGEDV | |
| | APGPLGLGQLLAVASQVAAGMVYLA | |
| | GLHFVHRDLATRNCLVGQGLVVKIG | |
| | DFGMSRDIYSTDYYRVGGRTMLPIR | |
| | WMPPESILYRKFTTESDVWSFGVVL | |
| | WEIFTYGKQPWYQLSNTEAIDCITQ | |
| | GRELERPRACPPEVYAIMRGCWQRE | |
| | PQQRHSIKDVHARLQALAQAPPVYL | |
| | DVLGSRGGGGSGGGGSGGGGSGGGG | |
| | SRSRFVKKDSAGSAGSAGSAGSYLA | |
| | NEILWGSAGSAGSAGSAGFCYENEV | |
| | ALS | |
| 24 | METDTLLLWVLLLWVPGSTGDSKGE | Fusion |
| | EDNMAIIKEFMRFKVHMEGSVNGHE | construct |
| | FEIEGEGEGRPYEGTQTAKLKVTKG | (TrkB) |
| | GPLPFAWDILSPQFMYGSKAYVKHP | |
| | ADIPDYLKLSFPEGFKWERVMNFED | |
| | GGVVTVTQDSSLQDGEFIYKVKLRG | |
| | TNFPSDGPVMQKKTMGWEASSERMY | |
| | PEDGALKGEIKQRLKLKDGGHYDAE | |
| | VKTTYKAKKPVQLPGAYNVNIKLDI | |
| | TSHNEDYTIVEQYERAEGRHSTGGM | |
| | DELYKEFSAGSAGSAGTGMSRDPLP | |
| | FFPPLYLGGPEITTENCEREPIHIP | |
| | GSIQPHGALLTADGHSGEVLQMSLN | |
| | AATFLGQEPTVLRGQTLAALLPEQW | |
| | PALQAALPPGCPDALQYRATLDWPA | |
| | AGHLSLTVHRVGELLILEFEPTEAW | |
| | DSTGPHALRNAMFAFESAPNLRALA | |
| | EVATQTVRELTGFDRVMLYKFAPDA | |
| | TGEVIAEARREGLHAFLGHRFPASD | |
| | IPAQARALYTRHLLRLTADTRAATV | |
| | PLDPVLNPQTNAPTPLGGAVLRATS | |
| | PMHMQYLRNMGVGSSLSVSVVVGGQ | |
| | LWGLIACHHQTPYVLPPDLRTTLEY | |
| | LGRLLSLQVQVKEAADVAAFRQSLR | |
| | EHHARVALAAAHSLSPHDTLSDPAL | |
| | DLLGLMRAGGLILRFEGRWQTLGEV | |
| | PPAPAVDALLAWLETQPGALVQTDA | |
| | LGQLWLAGADLAPSAAGLLAISVGE | |
| | GWSECLVWLRPELRLEVAWGGATPD | |
| | QAKDDLGPRHSFDTYLEEKRGYAEP | |
| | WHPGEIEEAQDLRDTLTGALGELEI | |
| | ATGMVGALLLLLVVALGIGLFMSRK | |
| | LARHSKFGMKGPASVISNDDDSASP | |
| | LHHISNGSNTPSSSEGGPDAVIIGM | |
| | TKIPVIENPQYFGITNSQLKPDTFV | |
| | QHIKRHNIVLKRELGEGAFGKVFLA | |
| | ECYNLCPEQDKILVAVKTLKDASDN | |
| | ARKDFHREAELLTNLQHEHIVKFYG | |
| | VCVEGDPLIMVFEYMKHGDLNKFLR | |
| | AHGPDAVLMAEGNPPTELTQSQMLH | |
| | IAQQIAAGMVYLASQHFVHRDLATR | |
| | NCLVGENLLVKIGDFGMSRDVYSTD | |
| | YYRVGGHTMLPIRWMPPESIMYRKF | |
| | TTESDVWSLGVVLWEIFTYGKQPWY | |
| | QLSNNEVIECITQGRVLQRPRTCPQ | |
| | EVYELMLGCWQREPHMRKNIKGIHT | |
| | LLQNLAKASPVYLDILGSRGGGGSG | |
| | GGGSGGGGSGGGGSRSRFVKKDSAG | |
| | SAGSAGSAGSYLANEILWGSAGSAG | |
| | SAGSAGFCYENEVALS | |
| 25 | METDTLLLWVLLLWVPGSTGDSKGE | Fusion |
| | EDNMAIIKEFMRFKVHMEGSVNGHE | construct |
| | FEIEGEGEGRPYEGTQTAKLKVTKG | (cKIT) |
| | GPLPFAWDILSPQFMYGSKAYVKHP | |
| | ADIPDYLKLSFPEGFKWERVMNFED | |
| | GGVVTVTQDSSLQDGEFIYKVKLRG | |
| | TNFPSDGPVMQKKTMGWEASSERMY | |
| | PEDGALKGEIKQRLKLKDGGHYDAE | |
| | VKTTYKAKKPVQLPGAYNVNIKLDI | |
| | TSHNEDYTIVEQYERAEGRHSTGGM | |
| | DELYKEFSAGSAGSAGTGMSRDPLP | |
| | FFPPLYLGGPEITTENCEREPIHIP | |
| | GSIQPHGALLTADGHSGEVLQMSLN | |
| | AATFLGQEPTVLRGQTLAALLPEQW | |
| | PALQAALPPGCPDALQYRATLDWPA | |
| | AGHLSLTVHRVGELLILEFEPTEAW | |
| | DSTGPHALRNAMFAFESAPNLRALA | |
| | EVATQTVRELTGFDRVMLYKFAPDA | |
| | TGEVIAEARREGLHAFLGHRFPASD | |
| | IPAQARALYTRHLLRLTADTRAATV | |
| | PLDPVLNPQTNAPTPLGGAVLRATS | |
| | PMHMQYLRNMGVGSSLSVSVVVGGQ | |
| | LWGLIACHHQTPYVLPPDLRTTLEY | |
| | LGRLLSLQVQVKEAADVAAFRQSLR | |
| | EHHARVALAAAHSLSPHDTLSDPAL | |
| | DLLGLMRAGGLILRFEGRWQTLGEV | |
| | PPAPAVDALLAWLETQPGALVQTDA | |
| | LGQLWLAGADLAPSAAGLLAISVGE | |
| | GWSECLVWLRPELRLEVAWGGATPD | |
| | QAKDDLGPRHSFDTYLEEKRGYAEP | |
| | WHPGEIEEAQDLRDTLTGALGELES | |
| | IVSAVVGILLVVVLGVVFGSRKYLQ | |
| | KPMYEVQWKVVEEINGNNYVYIDPT | |
| | QLPYDHKWEFPRNRLSFGKTLGAGA | |
| | FGKVVEATAYGLIKSDAAMTVAVKM | |
| | LKPSAHLTEREALMSELKVLSYLGN | |
| | HMNIVNLLGACTIGGPTLVITEYCC | |
| | YGDLLNFLRRKRDSFICSKQEDHAE | |
| | AALYKNLLHSKESSCSDSTNEYMDM | |
| | KPGVSYVVPTKADKRRSVRIGSYIE | |
| | RDVTPAIMEDDELALDLEDLLSFSY | |
| | QVAKGMAFLASKNCIHRDLAARNIL | |
| | LTHGRITKICDFGLARDIKNDSNYV | |
| | VKGNARLPVKWMAPESIFNCVYTFE | |
| | SDVWSYGIFLWELFSLGSSPYPGMP | |
| | VDSKFYKMIKEGFRMLSPEHAPAEM | |
| | YDIMKTCWDADPLKRPTFKQIVQLI | |
| | EKQISESTNHIYSNLANCSPNRQKP | |
| | VVDHSVRINSVGSTASSSQPLLVHD | |
| | DVSRGGGGSGGGGSGGGGSGGGGSR | |
| | SRFVKKDSAGSAGSAGSAGSYLANE | |
| | ILWGSAGSAGSAGSAGFCYENEVAL | |
| | S | |
| 26 | METDTLLLWVLLLWVPGSTGDSKGE | Fusion |
| | EDNMAIIKEFMRFKVHMEGSVNGHE | construct |
| | FEIEGEGEGRPYEGTQTAKLKVTKG | (cMet) |
| | GPLPFAWDILSPQFMYGSKAYVKHP | |
| | ADIPDYLKLSFPEGFKWERVMNFED | |
| | GGVVTVTQDSSLQDGEFIYKVKLRG | |
| | TNFPSDGPVMQKKTMGWEASSERMY | |
| | PEDGALKGEIKQRLKLKDGGHYDAE | |
| | VKTTYKAKKPVQLPGAYNVNIKLDI | |
| | TSHNEDYTIVEQYERAEGRHSTGGM | |
| | DELYKEFSAGSAGSAGTGMSRDPLP | |
| | FFPPLYLGGPEITTENCEREPIHIP | |
| | GSIQPHGALLTADGHSGEVLQMSLN | |
| | AATFLGQEPTVLRGQTLAALLPEQW | |
| | PALQAALPPGCPDALQYRATLDWPA | |
| | AGHLSLTVHRVGELLILEFEPTEAW | |
| | DSTGPHALRNAMFAFESAPNLRALA | |
| | EVATQTVRELTGFDRVMLYKFAPDA | |
| | TGEVIAEARREGLHAFLGHRFPASD | |
| | IPAQARALYTRHLLRLTADTRAATV | |
| | PLDPVLNPQTNAPTPLGGAVLRATS | |
| | PMHMQYLRNMGVGSSLSVSVVVGGQ | |
| | LWGLIACHHQTPYVLPPDLRTTLEY | |
| | LGRLLSLQVQVKEAADVAAFRQSLR | |
| | EHHARVALAAAHSLSPHDTLSDPAL | |
| | DLLGLMRAGGLILRFEGRWQTLGEV | |
| | PPAPAVDALLAWLETQPGALVQTDA | |
| | LGQLWLAGADLAPSAAGLLAISVGE | |
| | GWSECLVWLRPELRLEVAWGGATPD | |
| | QAKDDLGPRHSFDTYLEEKRGYAEP | |
| | WHPGEIEEAQDLRDTLTGALGEYFY | |
| | FSIVSAVVGILLVVVLGVVFGSRKK | |
| | RKQIKDLGSELVRYDARVHTPHLDR | |
| | LVSARSVSPTTEMVSNESVDYRATF | |
| | PEDQFPNSSQNGSCRQVQYPLTDMS | |
| | PILTSGDSDISSPLLQNTVHIDLSA | |
| | LNPELVQAVQHVVIGPSSLIVHFNE | |
| | VIGRGHFGCVYHGTLLDNDGKKIHC | |
| | AVKSLNRITDIGEVSQFLTEGIIMK | |
| | DFSHPNVLSLLGICLRSEGSPLVVL | |
| | PYMKHGDLRNFIRNETHNPTVKDLI | |
| | GFGLQVAKGMKYLASKKFVHRDLAA | |
| | RNCMLDEKFTVKVADFGLARDMYDK | |
| | EYYSVHNKTGAKLPVKWMALESLQT | |
| | QKFTTKSDVWSFGVLLWELMTRGAP | |
| | PYPDVNTFDITVYLLQGRRLLQPEY | |
| | CPDPLYEVMLKCWHPKAEMRPSFSE | |
| | LVSRISAIFSTFIGEHYVHVNATYV | |
| | NVKCVAPYPSLLSSEDNADDEVDTR | |
| | PASFWETSRGGGGSGGGGSGGGGSG | |
| | GGGSRSRFVKKDSAGSAGSAGSAGS | |
| | YLANEILWGSAGSAGSAGSAGFCYE | |
| | NEVALS | |
| 27 | METDTLLLWVLLLWVPGSTGDSKGE | Fusion |
| | EDNMAIIKEFMRFKVHMEGSVNGHE | construct |
| | FEIEGEGEGRPYEGTQTAKLKVTKG | (IR1) |
| | GPLPFAWDILSPQFMYGSKAYVKHP | |
| | ADIPDYLKLSFPEGFKWERVMNFED | |
| | GGVVTVTQDSSLQDGEFIYKVKLRG | |
| | TNFPSDGPVMQKKTMGWEASSERMY | |
| | PEDGALKGEIKQRLKLKDGGHYDAE | |
| | VKTTYKAKKPVQLPGAYNVNIKLDI | |
| | TSHNEDYTIVEQYERAEGRHSTGGM | |
| | DELYKEFSAGSAGSAGTGMSRDPLP | |
| | FFPPLYLGGPEITTENCEREPIHIP | |
| | GSIQPHGALLTADGHSGEVLQMSLN | |
| | AATFLGQEPTVLRGQTLAALLPEQW | |
| | PALQAALPPGCPDALQYRATLDWPA | |
| | AGHLSLTVHRVGELLILEFEPTEAW | |
| | DSTGPHALRNAMFAFESAPNLRALA | |
| | EVATQTVRELTGFDRVMLYKFAPDA | |
| | TGEVIAEARREGLHAFLGHRFPASD | |
| | IPAQARALYTRHLLRLTADTRAATV | |
| | PLDPVLNPQTNAPTPLGGAVLRATS | |
| | PMHMQYLRNMGVGSSLSVSVVVGGQ | |
| | LWGLIACHHQTPYVLPPDLRTTLEY | |
| | LGRLLSLQVQVKEAADVAAFRQSLR | |
| | EHHARVALAAAHSLSPHDTLSDPAL | |
| | DLLGLMRAGGLILRFEGRWQTLGEV | |
| | PPAPAVDALLAWLETQPGALVQTDA | |
| | LGQLWLAGADLAPSAAGLLAISVGE | |
| | GWSECLVWLRPELRLEVAWGGATPD | |
| | QAKDDLGPRHSFDTYLEEKRGYAEP | |
| | WHPGEIEEAQDLRDTLTGALGELES | |
| | IVSAVVGILLVVVLGVVFGSRRKRQ | |
| | PDGPLGPLYASSNPEYLSASDVFPC | |
| | SVYVPDEWEVSREKITLLRELGQGS | |
| | FGMVYEGNARDIIKGEAETRVAVKT | |
| | VNESASLRERIEFLNEASVMKGFTC | |
| | HHVVRLLGVVSKGQPTLVVMELMAH | |
| | GDLKSYLRSLRPEAENNPGRPPPTL | |
| | QEMIQMAAEIADGMAYLNAKKFVHR | |
| | DLAARNCMVAHDFTVKIGDFGMTRD | |
| | IYETDYYRKGGKGLLPVRWMAPESL | |
| | KDGVFTTSSDMWSFGVVLWEITSLA | |
| | EQPYQGLSNEQVLKFVMDGGYLDQP | |
| | DNCPERVTDLMRMCWQFNPKMRPTF | |
| | LEIVNLLKDDLHPSFPEVSFFHSEE | |
| | NKAPESEELEMEFEDMENVPLDRSS | |
| | HCQREEAGGRDGGSSLGFKRSYEEH | |
| | IPYTHMNGGKKNGRILTLPRSNPSS | |
| | RGGGGSGGGGSGGGGSGGGGSRSRF | |
| | VKKDSAGSAGSAGSAGSYLANEILW | |
| | GSAGSAGSAGSAGFCYENEVALS | |
| 28 | ATGAGCCGGGACCCGTTGCCCTTTT | DrBphP |
| | TTCCACCGCTTTACCTTGGTGGCCC | |
| | GGAAATTACCACCGAGAACTGCGAG | |
| | CGCGAGCCGATTCATATTCCCGGCA | |
| | GCATCCAGCCGCACGGCGCCCTGCT | |
| | CACTGCCGACGGGCACAGCGGCGAG | |
| | GTGCTCCAGATGAGCCTCAACGCGG | |
| | CCACTTTTCTGGGACAGGAACCCAC | |
| | AGTGCTGCGCGGACAGACCCTCGCC | |
| | GCACTGCTGCCCGAGCAGTGGCCCG | |
| | CGCTGCAAGCGGCCCTGCCCCCCGG | |
| | CTGCCCCGACGCCCTGCAATACCGC | |
| | GCAACGCTGGACTGGCCTGCCGCCG | |
| | GGCACCTTTCGCTGACGGTGCACCG | |
| | GGTCGGCGAGTTGCTGATTCTGGAA | |
| | TTCGAGCCGACGGAGGCCTGGGACA | |
| | GCACCGGGCCGCACGCGCTGCGCAA | |
| | CGCGATGTTCGCGCTCGAAAGTGCC | |
| | CCCAACCTGCGGGCGCTGGCCGAGG | |
| | TGGCGACCCAGACGGTCCGCGAGCT | |
| | GACGGGCTTTGACCGGGTGATGCTC | |
| | TACAAATTTGCCCCCGACGCCACCG | |
| | GCGAAGTGATTGCCGAGGCCCGCCG | |
| | TGAGGGGCTGCACGCCTTTCTGGGC | |
| | CACCGTTTTCCCGCGTCGGACATTC | |
| | CGGCGCAGGCCCGCGCGCTCTACAC | |
| | CCGGCACCTGCTGCGCCTGACCGCC | |
| | GACACCCGCGCCGCCGCCGTGCCGC | |
| | TCGATCCCGTCCTCAACCCGCAGAC | |
| | GAATGCGCCCACCCCGCTGGGCGGC | |
| | GCCGTGCTGCGCGCCACCTCGCCCA | |
| | TGCACATGCAGTACCTGCGGAACAT | |
| | GGGCGTCGGGTCGAGCCTGTCGGTG | |
| | TCGGTGGTGGTCGGCGGCCAGCTCT | |
| | GGGGCCTGATCGCCTGCCACCACCA | |
| | GACGCCCTACGTGTTGCCGCCCGAC | |
| | CTGCGAACCACGCTCGAATACCTGG | |
| | GCCGCTTGCTGAGCCTGCAAGTTCA | |
| | GGTCAAGGAAGCGGCGGACGTGGCG | |
| | GCCTTTCGCCAGAGCCTGCGGGAGC | |
| | ACCACGCGCGGGTGGCCCTCGCGGC | |
| | GGCGCACTCGCTCTCGCCGCACGAC | |
| | ACCCTCAGTGACCCGGCGCTTGACC | |
| | TGCTGGGCCTGATGCGGGCCGGGGG | |
| | CCTGATTCTGCGTTTCGAGGGCCGC | |
| | TGGCAGACGTTGGGTGAAGTGCCGC | |
| | CTGCCCCGGCGGTGGACGCGCTGCT | |
| | GGCGTGGCTCGAAACCCAGCCGGGC | |
| | GCCCTGGTCCAGACCGACGCGCTGG | |
| | GCCAACTGTGGCCCGCCGGCGCCGA | |
| | TCTCGCCCCCAGCGCAGCGGGCCTG | |
| | CTCGCCATCAGCGTGGGCGAGGGCT | |
| | GGTCGGAGTGCCTCGTCTGGCTGCG | |
| | GCCCGAACTGCGGCTGGAGGTCGCC | |
| | TGGGGCGGGGCCACTCCTGACCAGG | |
| | CGAAAGACGACCTCGGGCCGCGCCA | |
| | CTCATTCGACACCTACCTCGAAGAA | |
| | AAACGCGGCTACGCCGAGCCCTGGC | |
| | ATCCCGGCGAAATCGAGGAGGCGCA | |
| | GGATCTACGTGACACATTGACCGGG | |
| | GCGCTGGGCGAGCGCCTGAGCGTGA | |
| | TTCGTGACCTCAACCGGGCGCTCAC | |
| | ACAGTCGAACGCCGAGTGGCGGCAG | |
| | TACGGCTTCGTTATCAGCCACCACA | |
| | TGCAGGAGCCGGTGCGGCTCATCTC | |
| | GCAGTTCGCCGAGTTGCTGACGCGC | |
| | CAGCCCCGCGCCCAGGACGGGTCTC | |
| | CGGACTCTCCGCAGACCGAGCGCAT | |
| | CACCGGCTTTCTGCTGCGCGAAACG | |
| | TCGCGCCTGCGCAGCCTGACGCAAG | |
| | ACCTCCACACCTACACCGCGCTGCT | |
| | CTCGGCACCGCCGCCGGTGCGCCGC | |
| | CCCACGCCGCTGGGCCGCGTGGTGG | |
| | ACGATGTGCTGCAAGACCTCGAACC | |
| | CCGCATTGCCGACACCGGAGCGAGC | |
| | ATCGAGGTGGCGCCCGAGTTGCCCG | |
| | TCATCGCTGCCGACGCTGGCCTGCT | |
| | GCGCGACCTGCTGCTGCATCTGATC | |
| | GGCAACGCGCTGACGTTTGGTGGCC | |
| | CGGAGCCGCGTATTGCCGTAAGGAC | |
| | CGAACGGCAAGGCGCGGGTTGGTCT | |
| | ATCGCGGTCAGTGACCAGGGCGCTG | |
| | GCATCGCGCCCGAGTATCAGGAACG | |
| | AATCTTTCTGCTGTTTCAGCGGCTC | |
| | GGTTCGCTCGATGAGGCGCTGGGCA | |
| | ACGGCCTGGGCCTGCCGCTGTGCCG | |
| | CAAGATCGCCGAACTGCATGGCGGC | |
| | ACCCTGACCGTGGAGTCCGCGCCAG | |
| | GCGAGGGCAGCACCTTCCGTTGCTG | |
| | GCTGCCCGATGCTGGGCCTCTTCCG | |
| | GGAGCCGCCGATGCCTGA | |
| 29 | ATGAGCCGGGACCCGTTGCCCTTTT | DrBphP-PCM |
| | TTCCACCGCTTTACCTTGGTGGCCC | |
| | GGAAATTACCACCGAGAACTGCGAG | |
| | CGCGAGCCGATTCATATTCCCGGCA | |
| | GCATCCAGCCGCACGGCGCCCTGCT | |
| | CACTGCCGACGGGCACAGCGGCGAG | |
| | GTGCTCCAGATGAGCCTCAACGCGG | |
| | CCACTTTTTTGGGACAGGAACCCAC | |
| | AGTGCTGCGCGGACAGACCCTCGCC | |
| | GCACTGCTGCCCGAGCAGTGGCCCG | |
| | CGCTGCAAGCGGCCCTGCCCCCCGG | |
| | CTGCCCCGACGCCCTGCAATACCGC | |
| | GCAACGCTGGACTGGCCTGCCGCCG | |
| | GGCACCTTTCGCTGACGGTGCACCG | |
| | GGTCGGCGAGTTGCTGATTCTGGAG | |
| | TTCGAGCCGACGGAGGCCTGGGACA | |
| | GCACCGGGCCGCACGCGCTGCGCAA | |
| | CGCGATGTTCGCGTTCGAAAGTGCC | |
| | CCCAACCTGCGGGCGCTGGCCGAGG | |
| | TGGCGACCCAGACGGTCCGCGAGCT | |
| | GACGGGCTTTGACCGGGTGATGCTC | |
| | TACAAATTTGCCCCCGACGCCACCG | |
| | GCGAAGTGATTGCCGAGGCCCGCCG | |
| | TGAGGGGCTGCACGCCTTTCTGGGC | |
| | CACCGTTTTCCCGCGTCGGACATTC | |
| | CGGCGCAGGCCCGCGCGCTCTACAC | |
| | CCGGCACCTGCTGCGCCTGACCGCC | |
| | GACACCCGCGCCGCCACCGTGCCGC | |
| | TCGATCCCGTCCTCAACCCGCAGAC | |
| | GAATGCGCCCACCCCGCTGGGCGGC | |
| | GCCGTGCTGCGCGCCACCTCGCCCA | |
| | TGCACATGCAGTACCTGCGGAACAT | |
| | GGGCGTCGGGTCGAGCCTGTCGGTG | |
| | TCGGTGGTGGTCGGCGGCCAGCTCT | |
| | GGGGCCTGATCGCCTGCCACCACCA | |
| | GACGCCCTACGTGTTGCCGCCCGAC | |
| | CTGCGAACCACGCTCGAATACCTGG | |
| | GCCGCTTGCTGAGCCTGCAAGTTCA | |
| | GGTCAAGGAAGCGGCGGACGTGGCG | |
| | GCCTTTCGCCAGAGCCTGCGGGAGC | |
| | ACCACGCGCGGGTGGCCCTCGCGGC | |
| | GGCGCACTCGCTCTCGCCGCACGAC | |
| | ACCCTCAGTGACCCGGCGCTTGACC | |
| | TGCTGGGCCTGATGCGGGCCGGGGG | |
| | CCTGATTCTGCGTTTCGAGGGCCGC | |
| | TGGCAGACGTTGGGTGAAGTGCCGC | |
| | CTGCCCCGGCGGTGGACGCGCTGCT | |
| | GGCGTGGCTCGAAACCCAGCCGGGC | |
| | GCCCTGGTCCAGACCGACGCGCTAG | |
| | GCCAACTGTGGCTCGCCGGCGCCGA | |
| | TCTCGCCCCCAGCGCAGCGGGCCTG | |
| | CTCGCCATCAGCGTGGGCGAGGGCT | |
| | GGTCGGAGTGCCTCGTCTGGCTGCG | |
| | GCCCGAACTGCGGCTGGAGGTCGCC | |
| | TGGGGCGGGGCCACTCCTGACCAGG | |
| | CGAAAGACGACCTCGGGCCGCGCCA | |
| | CTCATTCGACACCTACCTCGAAGAA | |
| | AAACGCGGCTACGCCGAGCCCTGGC | |
| | ATCCCGGCGAAATCGAGGAGGCGCA | |
| | GGATCTACGTGACACATTGACCGGG | |
| | GCGCTGGGCGAG | |
| 30 | AAGGGCGAGGAGGATAACATGGCCA | DrBphP-PCM |
| | TCATCAAGGAGTTCATGCGCTTCAA | variant? |
| | GGTGCACATGGAGGGCTCCGTGAAC | |
| | GGCCACGAGTTCGAGATCGAGGGCG | |
| | AGGGCGAGGGCCGCCCCTACGAGGG | |
| | CACCCAGACCGCCAAGCTGAAGGTG | |
| | ACCAAGGGTGGCCCCCTGCCCTTCG | |
| | CCTGGGACATCCTGTCCCCTCAGTT | |
| | CATGTACGGCTCCAAGGCCTACGTG | |
| | AAGCACCCCGCCGACATCCCCGACT | |
| | ACTTGAAGCTGTCCTTCCCCGAGGG | |
| | CTTCAAGTGGGAGCGCGTGATGAAC | |
| | TTCGAGGACGGCGGCGTGGTGACCG | |
| | TGACCCAGGACTCCTCCCTGCAGGA | |
| | CGGCGAGTTCATCTACAAGGTGAAG | |
| | CTGCGCGGCACCAACTTCCCCTCCG | |
| | ACGGCCCCGTAATGCAGAAGAAGAC | |
| | CATGGGCTGGGAGGCCTCCTCCGAG | |
| | CGGATGTACCCCGAGGACGGCGCCC | |
| | TGAAGGGCGAGATCAAGCAGAGGCT | |
| | GAAGCTGAAGGACGGCGGCCACTAC | |
| | GACGCTGAGGTCAAGACCACCTACA | |
| | AGGCCAAGAAGCCCGTGCAGCTGCC | |
| | CGGCGCCTACAACGTCAACATCAAG | |
| | TTGGACATCACCTCCCACAACGAGG | |
| | ACTACACCATCGTGGAACAGTACGA | |
| | ACGCGCCGAGGGCCGCCACTCCACC | |
| | GGCGGCATGGACGAGCTGTACAAGG | |
| | AATTCAGTGCTGGTAGTGCTGGTAG | |
| | TGCTGGCACCGGTATGAGCCGGGAC | |
| | CCGTTGCCCTTTTTTCCACCGCTTT | |
| | ACCTTGGTGGCCCGGAAATTACCAC | |
| | CGAGAACTGCGAGCGCGAGCCGATT | |
| | CATATTCCCGGCAGCATCCAGCCGC | |
| | ACGGCGCCCTGCTCACTGCCGACGG | |
| | GCACAGCGGCGAGGTGCTCCAGATG | |
| | AGCCTCAACGCGGCCACTTTTTTGG | |
| | GACAGGAACCCACAGTGCTGCGCGG | |
| | ACAGACCCTCGCCGCACTGCTGCCC | |
| | GAGCAGTGGCCCGCGCTGCAAGCGG | |
| | CCCTGCCCCCCGGCTGCCCCGACGC | |
| | CCTGCAATACCGCGCAACGCTGGAC | |
| | TGGCCTGCCGCCGGGCACCTTTCGC | |
| | TGACGGTGCACCGGGTCGGCGAGTT | |
| | GCTGATTCTGGAGTTCGAGCCGACG | |
| | GAGGCCTGGGACAGCACCGGGCCGC | |
| | ACGCGCTGCGCAACGCGATGTTCGC | |
| | GTTCGAAAGTGCCCCCAACCTGCGG | |
| | GCGCTGGCCGAGGTGGCGACCCAGA | |
| | CGGTCCGCGAGCTGACGGGCTTTGA | |
| | CCGGGTGATGCTCTACAAATTTGCC | |
| | CCCGACGCCACCGGCGAAGTGATTG | |
| | CCGAGGCCCGCCGTGAGGGGCTGCA | |
| | CGCCTTTCTGGGCCACCGTTTTCCC | |
| | GCGTCGGACATTCCGGCGCAGGCCC | |
| | GCGCGCTCTACACCCGGCACCTGCT | |
| | GCGCCTGACCGCCGACACCCGCGCC | |
| | GCCACCGTGCCGCTCGATCCCGTCC | |
| | TCAACCCGCAGACGAATGCGCCCAC | |
| | CCCGCTGGGCGGCGCCGTGCTGCGC | |
| | GCCACCTCGCCCATGCACATGCAGT | |
| | ACCTGCGGAACATGGGCGTCGGGTC | |
| | GAGCCTGTCGGTGTCGGTGGTGGTC | |
| | GGCGGCCAGCTCTGGGGCCTGATCG | |
| | CCTGCCACCACCAGACGCCCTACGT | |
| | GTTGCCGCCCGACCTGCGAACCACG | |
| | CTCGAATACCTGGGCCGCTTGCTGA | |
| | GCCTGCAAGTTCAGGTCAAGGAAGC | |
| | GGCGGACGTGGCGGCCTTTCGCCAG | |
| | AGCCTGCGGGAGCACCACGCGCGGG | |
| | TGGCCCTCGCGGCGGCGCACTCGCT | |
| | CTCGCCGCACGACACCCTCAGTGAC | |
| | CCGGCGCTTGACCTGCTGGGCCTGA | |
| | TGCGGGCCGGGGGCCTGATTCTGCG | |
| | TTTCGAGGGCCGCTGGCAGACGTTG | |
| | GGTGAAGTGCCGCCTGCCCCGGCGG | |
| | TGGACGCGCTGCTGGCGTGGCTCGA | |
| | AACCCAGCCGGGCGCCCTGGTCCAG | |
| | ACCGACGCGCTAGGCCAACTGTGGC | |
| | TCGCCGGCGCCGATCTCGCCCCCAG | |
| | CGCAGCGGGCCTGCTCGCCATCAGC | |
| | GTGGGCGAGGGCTGGTCGGAGTGCC | |
| | TCGTCTGGCTGCGGCCCGAACTGCG | |
| | GCTGGAGGTCGCCTGGGGCGGGGCC | |
| | ACTCCTGACCAGGCGAAAGACGACC | |
| | TCGGGCCGCGCCACTCATTCGACAC | |
| | CTACCTCGAAGAAAAACGCGGCTAC | |
| | GCCGAGCCCTGGCATCCCGGCGAAA | |
| | TCGAGGAGGCGCAGGATCTACGTGA | |
| | CACATTGACCGGGGCGCTGGGCGAG | |
| 31 | ATCGCCACTGGGATGGTGGGGGCCC | Transmembrane |
| | TCCTCTTGCTGCTGGTGGTGGCCCT | domain |
| | GGGGATCGGCCTCTTCATG | (EGFR) |
| 32 | TCCATCGTCTCTGCGGTGGTTGGCA | Transmembrane |
| | TTCTGCTGGTCGTGGTCTTGGGGGT | domain |
| | GGTCTTTGGG | (HER2) |
| 33 | TACTTTTATTTCATCGCCACTGGGA | Transmembrane |
| | TGGTGGGGGCCCTCCTCTTGCTGCT | domain |
| | GGTGGTGGCCCTGGGGATCGGCCTC | (EGFR) with |
| | TTCATG | YF repeats |
| 34 | TACTTTTATTTCTCCATCGTCTCTG | Transmembran |
| | CGGTGGTTGGCATTCTGCTGGTCGT | domaine |
| | GGTCTTGGGGGTGGTCTTTGGG | (HER2) with |
| | | YF repeats |
| | CGAAGGCGCCACATCGTTCGGAAGC | Intracellular |
| | GCACGCTGCGGAGGCTGCTGCAGGA | domain |
| | GAGGGAGCTTGTGGAGCCTCTTACA | (EGFR) |
| | CCCAGTGGAGAAGCTCCCAACCAAG | |
| | CTCTCTTGAGGATCTTGAAGGAAAC | |
| | TGAATTCAAAAAGATCAAAGTGCTG | |
| | GGCTCCGGTGCGTTCGGCACGGTGT | |
| | ATAAGGGACTCTGGATCCCAGAAGG | |
| | TGAGAAAGTTAAAATTCCCGTCGCT | |
| | ATCAAGGAATTAAGAGAAGCAACAT | |
| | CTCCGAAAGCCAACAAGGAAATCCT | |
| | CGATGAAGCCTACGTGATGGCCAGC | |
| | GTGGACAACCCCCACGTGTGCCGCC | |
| | TGCTGGGCATCTGCCTCACCTCCAC | |
| | CGTGCAACTCATCACGCAGCTCATG | |
| | CCCTTCGGCTGCCTCCTGGACTATG | |
| | TCCGGGAACACAAAGACAATATTGG | |
| | CTCCCAGTACCTGCTCAACTGGTGT | |
| | GTGCAGATCGCAAAGGGCATGAACT | |
| | ACTTGGAGGACCGTCGCTTGGTGCA | |
| | CCGCGACCTGGCAGCCAGGAACGTA | |
| | CTGGTGAAAACACCGCAGCATGTCA | |
| | AGATCACAGATTTTGGGCTGGCCAA | |
| | ACTGCTGGGTGCGGAAGAGAAAGAA | |
| | TACCATGCAGAAGGAGGCAAAGTGC | |
| | CTATCAAGTGGATGGCATTGGAATC | |
| | AATTTTACACAGAATCTATACCCAC | |
| | CAGAGTGATGTCTGGAGCTACGGGG | |
| | TGACCGTTTGGGAGTTGATGACCTT | |
| | TGGATCCAAGCCATATGACGGAATC | |
| | CCTGCCAGCGAGATCTCCTCCATCC | |
| | TGGAGAAAGGAGAACGCCTCCCTCA | |
| | GCCACCCATATGTACCATCGATGTC | |
| | TACATGATCATGGTCAAGTGCTGGA | |
| | TGATAGACGCAGATAGTCGCCCAAA | |
| | GTTCCGTGAGTTGATCATCGAATTC | |
| | TCCAAAATGGCCCGAGACCCCCAGC | |
| | GCTACCTTGTCATTCAGGGGGATGA | |
| | AAGAATGCATTTGCCAAGTCCTACA | |
| | GACTCCAACTTCTACCGTGCCCTGA | |
| | TGGATGAAGAAGACATGGACGACGT | |
| | GGTGGATGCCGACGAGTACCTCATC | |
| | CCACAGCAGGGCTTCTTCAGCAGCC | |
| | CCTCCACGTCACGGACTCCCCTCCT | |
| | GAGCTCTCTGAGTGCAACCAGCAAC | |
| | AATTCCACCGTGGCTTGCATTGATA | |
| | GAAATGGGCTGCAAAGCTGTCCCAT | |
| | CAAGGAAGACAGCTTCTTGCAGCGA | |
| | TACAGCTCAGACCCCACAGGCGCCT | |
| | TGACTGAGGACAGCATAGACGACAC | |
| | CTTCCTCCCAGTGCCTGAATACATA | |
| | AACCAGTCCGTTCCCAAAAGGCCCG | |
| | CTGGCTCTGTGCAGAATCCTGTCTA | |
| | TCACAATCAGCCTCTGAACCCCGCG | |
| | CCCAGCAGAGACCCACACTACCAGG | |
| | ACCCCCACAGCACTGCAGTGGGCAA | |
| | CCCCGAGTATCTCAACACTGTCCAG | |
| | CCCACCTGTGTCAACAGCACATTCG | |
| | ACAGCCCTGCCCACTGGGCCCAGAA | |
| | AGGCAGCCACCAAATTAGCCTGGAC | |
| | AACCCTGACTACCAGCAGGACTTCT | |
| | TTCCCAAGGAAGCCAAGCCAAATGG | |
| | CATCTTTAAGGGCTCCACAGCTGAA | |
| | AATGCAGAATACCTAAGGGTCGCGC | |
| | CACAAAGCAGTGAATTTATTGGAGC | |
| | A | |
| 35 | TCCATCATCTCTGCGGTGGTTGGCA | Intracellular |
| | TTCTGCTGGTCGTGGTCTTGGGGGT | domain |
| | GGTCTTTGGGATCCTCATCAAGCGA | (HER2) |
| | CGGCAGCAGAAGATCCGGAAGTACA | |
| | CGATGCGGAGACTGCTGCAGGAAAC | |
| | GGAGCTGGTGGAGCCGCTGACACCT | |
| | AGCGGAGCGATGCCCAACCAGGCGC | |
| | AGATGCGGATCCTGAAAGAGACGGA | |
| | GCTGAGGAAGGTGAAGGTGCTTGGA | |
| | TCTGGCGCTTTTGGCACAGTCTACA | |
| | AGGGCATCTGGATCCCTGATGGGGA | |
| | GAATGTGAAAATTCCAGTGGCCATC | |
| | AAAGTGTTGAGGGAAAACACATCCC | |
| | CCAAAGCCAACAAAGAAATCTTAGA | |
| | CGAAGCATACGTGATGGCTGGTGTG | |
| | GGCTCCCCATATGTCTCCCGCCTTC | |
| | TGGGCATCTGCCTGACATCCACGGT | |
| | GCAGCTGGTGACACAGCTTATGCCC | |
| | TATGGCTGCCTCTTAGACCATGTCC | |
| | GGGAAAACCGCGGACGCCTGGGCTC | |
| | CCAGGACCTGCTGAACTGGTGTATG | |
| | CAGATTGCCAAGGGGATGAGCTACC | |
| | TGGAGGATGTGCGGCTCGTACACAG | |
| | GGACTTGGCCGCTCGGAACGTGCTG | |
| | GTCAAGAGTCCCAACCATGTCAAAA | |
| | TTACAGACTTCGGGCTGGCTCGGCT | |
| | GCTGGACATTGACGAGACAGAGTAC | |
| | CATGCAGATGGGGGCAAGGTGCCCA | |
| | TCAAGTGGATGGCGCTGGAGTCCAT | |
| | TCTCCGCCGGCGGTTCACCCACCAG | |
| | AGTGATGTGTGGAGTTATGGTGTGA | |
| | CTGTGTGGGAGCTGATGACTTTTGG | |
| | GGCCAAACCTTACGATGGGATCCCA | |
| | GCCCGGGAGATCCCTGACCTGCTGG | |
| | AAAAGGGGGAGCGGCTGCCCCAGCC | |
| | CCCCATCTGCACCATTGATGTCTAC | |
| | ATGATCATGGTCAAATGTTGGATGA | |
| | TTGACTCTGAATGTCGGCCAAGATT | |
| | CCGGGAGTTGGTGTCTGAATTCTCC | |
| | CGCATGGCCAGGGACCCCCAGCGCT | |
| | TTGTGGTCATCCAGAATGAGGACTT | |
| | GGGCCCAGCCAGTCCCTTGGACAGC | |
| | ACCTTCTACCGCTCACTGCTGGAGG | |
| | ACGATGACATGGGGGACCTGGTGGA | |
| | TGCTGAGGAGTATCTGGTACCCCAG | |
| | CAGGGCTTCTTCTGTCCAGACCCTG | |
| | CCCCGGGCGCTGGGGGCATGGTCCA | |
| | CCACAGGCACCGCAGCTCATCTACC | |
| | AGGAGTGGCGGTGGGGACCTGACAC | |
| | TAGGGCTGGAGCCCTCTGAAGAGGA | |
| | GGCCCCCAGGTCTCCACTGGCACCC | |
| | TCCGAAGGGGCTGGCTCCGATGTAT | |
| | TTGATGGTGACCTGGGAATGGGGGC | |
| | AGCCAAGGGGCTGCAAAGCCTCCCC | |
| | ACACATGACCCCAGCCCTCTACAGC | |
| | GGTACAGTGAGGACCCCACAGTACC | |
| | CCTGCCCTCTGAGACTGATGGCTAC | |
| | GTTGCCCCCCTGACCTGCAGCCCCC | |
| | AGCCTGAATATGTGAACCAGCCAGA | |
| | TGTTCGGCCCCAGCCCCCTTCGCCC | |
| | CGAGAGGGCCCTCTGCCTGCTGCCC | |
| | GACCTGCTGGTGCCACTCTGGAAAG | |
| | GCCCAAGACTCTCTCCCCAGGGAAG | |
| | AATGGGGTCGTCAAAGACGTTTTTG | |
| | CCTTTGGGGGTGCCGTGGAGAACCC | |
| | CGAGTACTTGACACCCCAGGGAGGA | |
| | GCTGCCCCTCAGCCCCACCCTCCTC | |
| | CTGCCTTCAGCCCAGCCTTCGACAA | |
| | CCTCTATTACTGGGACCAGGACCCA | |
| | CCAGAGCGGGGGGCTCCACCCAGCA | |
| | CCTTCAAAGGGACACCTACGGCAGA | |
| | GAACCCAGAGTACCTGGGTCTGGAC | |
| | GTGCCAGTG | |
| 36 | AAGATGAAGAGTGGTACCAAGAAGA | Intracellular |
| | GTGACTTCCACAGCCAGATGGCTGT | domain |
| | GCACAAGCTGGCCAAGAGCATCCCT | (FGFR1) |
| | CTGCGCAGACAGGTAACAGTGTCTG | |
| | CTGACTCCAGTGCATCCATGAACTC | |
| | TGGGGTTCTTCTGGTTCGGCCATCA | |
| | CGGCTCTCCTCCAGTGGGACTCCCA | |
| | TGCTAGCAGGGGTCTCTGAGTATGA | |
| | GCTTCCCGAAGACCTTCGCTGGGAG | |
| | CTGCCTCGGGACAGACTGGTCTTAG | |
| | GCAAACCCCTGGGAGAGGGCTGCTT | |
| | TGGGCAGGTGGTGTTGGCAGAGGCT | |
| | ATCGGGCTGGACAAGGACAAACCCA | |
| | ACCGTGTGACCAAAGTGGCTGTGAA | |
| | GATGTTGAAGTCGGACGCAACAGAG | |
| | AAAGACTTGTCAGACCTGATCTCAG | |
| | AAATGGAGATGATGAAGATGATCGG | |
| | GAAGCATAAGAATATCATCAACCTG | |
| | CTGGGGGCCTGCACGCAGGATGGTC | |
| | CCTTGTATGTCATCGTGGAGTATGC | |
| | CTCCAAGGGCAACCTGCGGGAGTAC | |
| | CTGCAGGCCCGGAGGCCCCCAGGGC | |
| | TGGAATACTGCTACAACCCCAGCCA | |
| | CAACCCAGAGGAGCAGCTCTCCTCC | |
| | AAGGACCTGGTGTCCTGCGCCTACC | |
| | AGGTGGCCCGAGGCATGGAGTATCT | |
| | GGCCTCCAAGAAGTGCATACACCGA | |
| | GACCTGGCAGCCAGGAATGTCCTGG | |
| | TGACAGAGGACAATGTGATGAAGAT | |
| | AGCAGACTTTGGCCTCGCACGGGAC | |
| | ATTCACCACATCGACTACTATAAAA | |
| | AGACAACCAACGGCCGACTGCCTGT | |
| | GAAGTGGATGGCACCCGAGGCATTA | |
| | TTTGACCGGATCTACACCCACCAGA | |
| | GTGATGTGTGGTCTTTCGGGGTGCT | |
| | CCTGTGGGAGATCTTCACTCTGGGC | |
| | GGCTCCCCATACCCCGGTGTGCCTG | |
| | TGGAGGAACTTTTCAAGCTGCTGAA | |
| | GGAGGGTCACCGCATGGACAAGCCC | |
| | AGTAACTGCACCAACGAGCTGTACA | |
| | TGATGATGCGGGACTGCTGGCATGC | |
| | AGTGCCCTCACAGAGACCCACCTTC | |
| | AAGCAGCTGGTGGAAGACCTGGACC | |
| | GCATCGTGGCCTTGACCTCCAACCA | |
| | GGAGTACCTGGACCTGTCCATGCCC | |
| | CTGGACCAGTACTCCCCCAGCTTTC | |
| | CCGACACCCGGAGCTCTACGTGCTC | |
| | CTCAGGGGAGGATTCCGTCTTCTCT | |
| | CATGAGCCGCTGCCCGAGGAGCCCT | |
| | GCCTGCCCCGACACCCAGCCCAGCT | |
| | TGCCAATGGCGGACTCAAACGCCGC | |
| 37 | AACAAATGTGGACGGAGAAACAAGT | Intracellular |
| | TTGGGATCAACCGCCCGGCTGTGCT | domain (TrkA) |
| | GGCTCCAGAGGATGGGCTGGCCATG | |
| | TCCCTGCATTTCATGACATTGGGTG | |
| | GCAGCTCCCTGTCCCCCACCGAGGG | |
| | CAAAGGCTCTGGGCTCCAAGGCCAC | |
| | ATCATCGAGAACCCACAATACTTCA | |
| | GTGATGCCTGTGTTCACCACATCAA | |
| | GCGCCGGGACATCGTGCTCAAGTGG | |
| | GAGCTGGGGGAGGGCGCCTTTGGGA | |
| | AGGTCTTCCTTGCTGAGTGCCACAA | |
| | CCTCCTGCCTGAGCAGGACAAGATG | |
| | CTGGTGGCTGTCAAGGCACTGAAGG | |
| | AGGCGTCCGAGAGTGCTCGGCAGGA | |
| | CTTCCAACGTGAGGCTGAGCTGCTC | |
| | ACCATGCTGCAGCACCAGCACATCG | |
| | TGCGCTTCTTCGGCGTCTGCACCGA | |
| | GGGCCGCCCCCTGCTCATGGTCTTC | |
| | GAGTATATGCGGCACGGGGACCTCA | |
| | ACCGCTTCCTCCGATCCCATGGACC | |
| | CGATGCCAAGCTGCTGGCTGGTGGG | |
| | GAGGATGTGGCTCCAGGCCCCCTGG | |
| | GTCTGGGGCAGCTGCTGGCCGTGGC | |
| | TAGCCAGGTCGCTGCGGGGATGGTG | |
| | TACCTGGCGGGTCTGCATTTTGTGC | |
| | ACCGGGACCTGGCCACACGCAACTG | |
| | TCTAGTGGGCCAGGGACTGGTGGTC | |
| | AAGATTGGTGATTTTGGCATGAGCA | |
| | GGGATATCTACAGCACCGACTATTA | |
| | CCGTGTGGGAGGCCGCACCATGCTG | |
| | CCCATTCGCTGGATGCCGCCCGAGA | |
| | GCATCCTGTACCGTAAGTTCACCAC | |
| | CGAGAGCGACGTGTGGAGCTTCGGC | |
| | GTGGTGCTCTGGGAGATCTTCACCT | |
| | ACGGCAAGCAGCCCTGGTACCAGCT | |
| | CTCCAACACGGAGGCAATCGACTGC | |
| | ATCACGCAGGGACGTGAGTTGGAGC | |
| | GGCCACGTGCCTGCCCACCAGAGGT | |
| | CTACGCCATCATGCGGGGCTGCTGG | |
| | CAGCGGGAGCCCCAGCAACGCCACA | |
| | GCATCAAGGATGTGCACGCCCGGCT | |
| | GCAAGCCCTGGCCCAGGCACCTCCT | |
| | GTCTACCTGGATGTCCTGGGC | |
| | AAGTTGGCAAGACACTCCAAGTTTG | Intracellular |
| | GCATGAAAGGCCCAGCCTCCGTTAT | domain (TrkB) |
| | CAGCAATGATGATGACTCTGCCAGC | |
| | CCACTCCATCACATCTCCAATGGGA | |
| | GTAACACTCCATCTTCTTCGGAAGG | |
| | TGGCCCAGATGCTGTCATTATTGGA | |
| | ATGACCAAGATCCCTGTCATTGAAA | |
| | ATCCCCAGTACTTTGGCATCACCAA | |
| | CAGTCAGCTCAAGCCAGACACATTT | |
| | GTTCAGCACATCAAGCGACATAACA | |
| | TTGTTCTGAAAAGGGAGCTAGGCGA | |
| | AGGAGCCTTTGGAAAAGTGTTCCTA | |
| | GCTGAATGCTATAACCTCTGTCCTG | |
| | AGCAGGACAAGATCTTGGTGGCAGT | |
| | GAAGACCCTGAAGGATGCCAGTGAC | |
| | AATGCACGCAAGGACTTCCACCGTG | |
| | AGGCCGAGCTCCTGACCAACCTCCA | |
| | GCATGAGCACATCGTCAAGTTCTAT | |
| | GGCGTCTGCGTGGAGGGCGACCCCC | |
| | TCATCATGGTCTTTGAGTACATGAA | |
| | GCATGGGGACCTCAACAAGTTCCTC | |
| | AGGGCACACGGCCCTGATGCCGTGC | |
| | TGATGGCTGAGGGCAACCCGCCCAC | |
| | GGAACTGACGCAGTCGCAGATGCTG | |
| | CATATAGCCCAGCAGATCGCCGCGG | |
| | GCATGGTCTACCTGGCGTCCCAGCA | |
| | CTTCGTGCACCGCGATTTGGCCACC | |
| | AGGAACTGCCTGGTCGGGGAGAACT | |
| | TGCTGGTGAAAATCGGGGACTTTGG | |
| | GATGTCCCGGGACGTGTACAGCACT | |
| | GACTACTACAGGGTCGGTGGCCACA | |
| | CAATGCTGCCCATTCGCTGGATGCC | |
| | TCCAGAGAGCATCATGTACAGGAAA | |
| | TTCACGACGGAAAGCGACGTCTGGA | |
| | GCCTGGGGGTCGTGTTGTGGGAGAT | |
| | TTTCACCTATGGCAAACAGCCCTGG | |
| | TACCAGCTGTCAAACAATGAGGTGA | |
| | TAGAGTGTATCACTCAGGGCCGAGT | |
| | CCTGCAGCGACCCCGCACGTGCCCC | |
| | CAGGAGGTGTATGAGCTGATGCTGG | |
| | GGTGCTGGCAGCGAGAGCCCCACAT | |
| | GAGGAAGAACATCAAGGGCATCCAT | |
| | ACCCTCCTTCAGAACTTGGCCAAGG | |
| | CATCTCCGGTCTACCTGGACATTCT | |
| | AGGCTAG | |
| 38 | AAATATTTACAGAAACCCATGTATG | Intracellular |
| | AAGTACAGTGGAAGGTTGTTGAGGA | domain (cKIT) |
| | GATAAATGGAAACAATTATGTTTAC | |
| | ATAGACCCAACACAACTTCCTTATG | |
| | ATCACAAATGGGAGTTTCCCAGAAA | |
| | CAGGCTGAGTTTTGGGAAAACCCTG | |
| | GGTGCTGGAGCTTTCGGGAAGGTTG | |
| | TTGAGGCAACTGCTTATGGCTTAAT | |
| | TAAGTCAGATGCGGCCATGACTGTC | |
| | GCTGTAAAGATGCTCAAGCCGAGTG | |
| | CCCATTTGACAGAACGGGAAGCCCT | |
| | CATGTCTGAACTCAAAGTCCTGAGT | |
| | TACCTTGGTAATCACATGAATATTG | |
| | TGAATCTACTTGGAGCCTGCACCAT | |
| | TGGAGGGCCCACCCTGGTCATTACA | |
| | GAATATTGTTGCTATGGTGATCTTT | |
| | TGAATTTTTTGAGAAGAAAACGTGA | |
| | TTCATTTATTTGTTCAAAGCAGGAA | |
| | GATCATGCAGAAGCTGCACTTTATA | |
| | AGAATCTTCTGCATTCAAAGGAGTC | |
| | TTCCTGCAGCGATAGTACTAATGAG | |
| | TACATGGACATGAAACCTGGAGTTT | |
| | CTTATGTTGTCCCAACCAAGGCCGA | |
| | CAAAAGGAGATCTGTGAGAATAGGC | |
| | TCATACATAGAAAGAGATGTGACTC | |
| | CCGCCATCATGGAGGATGACGAGTT | |
| | GGCCCTAGACTTAGAAGACTTGCTG | |
| | AGCTTTTCTTACCAGGTGGCAAAGG | |
| | GCATGGCTTTCCTCGCCTCCAAGAA | |
| | TTGTATTCACAGAGACTTGGCAGCC | |
| | AGAAATATCCTCCTTACTCATGGTC | |
| | GGATCACAAAGATTTGTGATTTTGG | |
| | TCTAGCCAGAGACATCAAGAATGAT | |
| | TCTAATTATGTGGTTAAAGGAAACG | |
| | CTCGACTACCTGTGAAGTGGATGGC | |
| | ACCTGAAAGCATTTTCAACTGTGTA | |
| | TACACGTTTGAAAGTGACGTCTGGT | |
| | CCTATGGGATTTTTCTTTGGGAGCT | |
| | GTTCTCTTTAGGAAGCAGCCCCTAT | |
| | CCTGGAATGCCGGTCGATTCTAAGT | |
| | TCTACAAGATGATCAAGGAAGGCTT | |
| | CCGGATGCTCAGCCCTGAACACGCA | |
| | CCTGCTGAAATGTATGACATAATGA | |
| | AGACTTGCTGGGATGCAGATCCCCT | |
| | AAAAAGACCAACATTCAAGCAAATT | |
| | GTTCAGCTAATTGAGAAGCAGATTT | |
| | CAGAGAGCACCAATCATATTTACTC | |
| | CAACTTAGCAAACTGCAGCCCCAAC | |
| | CGACAGAAGCCCGTGGTAGACCATT | |
| | CTGTGCGGATCAATTCTGTCGGCAG | |
| | CACCGCTTCCTCCTCCCAGCCTCTG | |
| | CTTGTGCACGACGATGTC | |
| 39 | AAAAAGAGAAAGCAAATTAAAGATC | Intracellular |
| | TGGGCAGTGAATTAGTTCGCTACGA | domain (cMet) |
| | TGCAAGAGTACACACTCCTCATTTG | |
| | GATAGGCTTGTAAGTGCCCGAAGTG | |
| | TAAGCCCAACTACAGAAATGGTTTC | |
| | AAATGAATCTGTAGACTACCGAGCT | |
| | ACTTTTCCAGAAGATCAGTTTCCTA | |
| | ATTCATCTCAGAACGGTTCATGCCG | |
| | ACAAGTGCAGTATCCTCTGACAGAC | |
| | ATGTCCCCCATCCTAACTAGTGGGG | |
| | ACTCTGATATATCCAGTCCATTACT | |
| | GCAAAATACTGTCCACATTGACCTC | |
| | AGTGCTCTAAATCCAGAGCTGGTCC | |
| | AGGCAGTGCAGCATGTAGTGATTGG | |
| | GCCCAGTAGCCTGATTGTGCATTTC | |
| | AATGAAGTCATAGGAAGAGGGCATT | |
| | TTGGTTGTGTATATCATGGGACTTT | |
| | GTTGGACAATGATGGCAAGAAAATT | |
| | CACTGTGCTGTGAAATCCTTGAACA | |
| | GAATCACTGACATAGGAGAAGTTTC | |
| | CCAATTTCTGACCGAGGGAATCATC | |
| | ATGAAAGATTTTAGTCATCCCAATG | |
| | TCCTCTCGCTCCTGGGAATCTGCCT | |
| | GCGAAGTGAAGGGTCTCCGCTGGTG | |
| | GTCCTACCATACATGAAACATGGAG | |
| | ATCTTCGAAATTTCATTCGAAATGA | |
| | GACTCATAATCCAACTGTAAAAGAT | |
| | CTTATTGGCTTTGGTCTTCAAGTAG | |
| | CCAAAGGCATGAAATATCTTGCAAG | |
| | CAAAAAGTTTGTCCACAGAGACTTG | |
| | GCTGCAAGAAACTGTATGCTGGATG | |
| | AAAAATTCACAGTCAAGGTTGCTGA | |
| | TTTTGGTCTTGCCAGAGACATGTAT | |
| | GATAAAGAATACTATAGTGTACACA | |
| | ACAAAACAGGTGCAAAGCTGCCAGT | |
| | GAAGTGGATGGCTTTGGAAAGTCTG | |
| | CAAACTCAAAAGTTTACCACCAAGT | |
| | CAGATGTGTGGTCCTTTGGCGTGCT | |
| | CCTCTGGGAGCTGATGACAAGAGGA | |
| | GCCCCACCTTATCCTGACGTAAACA | |
| | CCTTTGATATAACTGTTTACTTGTT | |
| | GCAAGGGAGAAGACTCCTACAACCC | |
| | GAATACTGCCCAGACCCCTTATATG | |
| | AAGTAATGCTAAAATGCTGGCACCC | |
| | TAAAGCCGAAATGCGCCCATCCTTT | |
| | TCTGAACTGGTGTCCCGGATATCAG | |
| | CGATCTTCTCTACTTTCATTGGGGA | |
| | GCACTATGTCCATGTGAACGCTACT | |
| | TATGTGAACGTAAAATGTGTCGCTC | |
| | CGTATCCTTCTCTGTTGTCATCAGA | |
| | AGATAACGCTGATGATGAGGTGGAC | |
| | ACACGACCAGCCTCCTTCTGGGAGA | |
| | CATCA | |
| 40 | AGAAAGAGGCAGCCAGATGGGCCGC | Intracellular |
| | TGGGACCGCTTTACGCTTCTTCAAA | domain (IR1) |
| | CCCTGAGTATCTCAGTGCCAGTGAT | |
| | GTGTTTCCATGCTCTGTGTACGTGC | |
| | CGGACGAGTGGGAGGTGTCTCGAGA | |
| | GAAGATCACCCTCCTTCGAGAGCTG | |
| | GGGCAGGGCTCCTTCGGCATGGTGT | |
| | ATGAGGGCAATGCCAGGGACATCAT | |
| | CAAGGGTGAGGCAGAGACCCGCGTG | |
| | GCGGTGAAGACGGTCAACGAGTCAG | |
| | CCAGTCTCCGAGAGCGGATTGAGTT | |
| | CCTCAATGAGGCCTCGGTCATGAAG | |
| | GGCTTCACCTGCCATCACGTGGTGC | |
| | GCCTCCTGGGAGTGGTGTCCAAGGG | |
| | CCAGCCCACGCTGGTGGTGATGGAG | |
| | CTGATGGCTCACGGAGACCTGAAGA | |
| | GCTACCTCCGTTCTCTGCGGCCAGA | |
| | GGCTGAGAATAATCCTGGCCGCCCT | |
| | CCCCCTACCCTTCAAGAGATGATTC | |
| | AGATGGCGGCAGAGATTGCTGACGG | |
| | GATGGCCTACCTGAACGCCAAGAAG | |
| | TTTGTGCATCGGGACCTGGCAGCGA | |
| | GAAACTGCATGGTCGCCCATGATTT | |
| | TACTGTCAAAATTGGAGACTTTGGA | |
| | ATGACCAGAGACATCTATGAAACGG | |
| | ATTACTACCGGAAAGGGGGCAAGGG | |
| | TCTGCTCCCTGTACGGTGGATGGCA | |
| | CCGGAGTCCCTGAAGGATGGGGTCT | |
| | TCACCACTTCTTCTGACATGTGGTC | |
| | CTTTGGCGTGGTCCTTTGGGAAATC | |
| | ACCAGCTTGGCAGAACAGCCTTACC | |
| | AAGGCCTGTCTAATGAACAGGTGTT | |
| | GAAATTTGTCATGGATGGAGGGTAT | |
| | CTGGATCAACCCGACAACTGTCCAG | |
| | AGAGAGTCACTGACCTCATGCGCAT | |
| | GTGCTGGCAATTCAACCCCAAGATG | |
| | AGGCCAACCTTCCTGGAGATTGTCA | |
| | ACCTGCTCAAGGACGACCTGCACCC | |
| | CAGCTTTCCAGAGGTGTCGTTCTTC | |
| | CACAGCGAGGAGAACAAGGCTCCCG | |
| | AGAGTGAGGAGCTGGAGATGGAGTT | |
| | TGAGGACATGGAGAATGTGCCCCTG | |
| | GACCGTTCCTCGCACTGTCAGAGGG | |
| | AGGAGGCGGGGGGCCGGGATGGAGG | |
| | GTCCTCGCTGGGTTTCAAGCGGAGC | |
| | TACGAGGAACACATCCCTTACACAC | |
| | ACATGAACGGAGGCAAGAAAAACGG | |
| | GCGGATTCTGACCTTGCCTCGGTCC | |
| | AATCCTTCCTAA | |
| 41 | ATGGAGACAGACACACTCCTGCTAT | Signaling |
| | GGGTACTGCTGCTCTGGGTTCCAGG | peptide Ig κ |
| | TTCCACTGGTGACAGC | |
| 42 | CGCAGCCGCTTTGTGAAAAAAGAT | Golgi-export |
| | | sequence 1 |
| 43 | AGCTATCTGGCGAACGAAATTCTGT | Golgi-export |
| | GG | sequence 2 |
| 44 | TTTTGCTATGAAAACGAAGTGGCGC | ER-export |
| | TGTCA | sequence |
| 45 | ATGGAGACAGACACACTCCTGCTAT | Fusion |
| | GGGTACTGCTGCTCTGGGTTCCAGG | construct |
| | TTCCACTGGTGACAGCAAGGGCGAG | (EGFR) |
| | GAGGATAACATGGCCATCATCAAGG | |
| | AGTTCATGCGCTTCAAGGTGCACAT | |
| | GGAGGGCTCCGTGAACGGCCACGAG | |
| | TTCGAGATCGAGGGCGAGGGCGAGG | |
| | GCCGCCCCTACGAGGGCACCCAGAC | |
| | CGCCAAGCTGAAGGTGACCAAGGGT | |
| | GGCCCCCTGCCCTTCGCCTGGGACA | |
| | TCCTGTCCCCTCAGTTCATGTACGG | |
| | CTCCAAGGCCTACGTGAAGCACCCC | |
| | GCCGACATCCCCGACTACTTGAAGC | |
| | TGTCCTTCCCCGAGGGCTTCAAGTG | |
| | GGAGCGCGTGATGAACTTCGAGGAC | |
| | GGCGGCGTGGTGACCGTGACCCAGG | |
| | ACTCCTCCCTGCAGGACGGCGAGTT | |
| | CATCTACAAGGTGAAGCTGCGCGGC | |
| | ACCAACTTCCCCTCCGACGGCCCCG | |
| | TAATGCAGAAGAAGACCATGGGCTG | |
| | GGAGGCCTCCTCCGAGCGGATGTAC | |
| | CCCGAGGACGGCGCCCTGAAGGGCG | |
| | AGATCAAGCAGAGGCTGAAGCTGAA | |
| | GGACGGCGGCCACTACGACGCTGAG | |
| | GTCAAGACCACCTACAAGGCCAAGA | |
| | AGCCCGTGCAGCTGCCCGGCGCCTA | |
| | CAACGTCAACATCAAGTTGGACATC | |
| | ACCTCCCACAACGAGGACTACACCA | |
| | TCGTGGAACAGTACGAACGCGCCGA | |
| | GGGCCGCCACTCCACCGGCGGCATG | |
| | GACGAGCTGTACAAGGAATTCAGTG | |
| | CTGGTAGTGCTGGTAGTGCTGGCAC | |
| | CGGTATGAGCCGGGACCCGTTGCCC | |
| | TTTTTTCCACCGCTTTACCTTGGTG | |
| | GCCCGGAAATTACCACCGAGAACTG | |
| | CGAGCGCGAGCCGATTCATATTCCC | |
| | GGCAGCATCCAGCCGCACGGCGCCC | |
| | TGCTCACTGCCGACGGGCACAGCGG | |
| | CGAGGTGCTCCAGATGAGCCTCAAC | |
| | GCGGCCACTTTTTTGGGACAGGAAC | |
| | CCACAGTGCTGCGCGGACAGACCCT | |
| | CGCCGCACTGCTGCCCGAGCAGTGG | |
| | CCCGCGCTGCAAGCGGCCCTGCCCC | |
| | CCGGCTGCCCCGACGCCCTGCAATA | |
| | CCGCGCAACGCTGGACTGGCCTGCC | |
| | GCCGGGCACCTTTCGCTGACGGTGC | |
| | ACCGGGTCGGCGAGTTGCTGATTCT | |
| | GGAGTTCGAGCCGACGGAGGCCTGG | |
| | GACAGCACCGGGCCGCACGCGCTGC | |
| | GCAACGCGATGTTCGCGTTCGAAAG | |
| | TGCCCCCAACCTGCGGGCGCTGGCC | |
| | GAGGTGGCGACCCAGACGGTCCGCG | |
| | AGCTGACGGGCTTTGACCGGGTGAT | |
| | GCTCTACAAATTTGCCCCCGACGCC | |
| | ACCGGCGAAGTGATTGCCGAGGCCC | |
| | GCCGTGAGGGGCTGCACGCCTTTCT | |
| | GGGCCACCGTTTTCCCGCGTCGGAC | |
| | ATTCCGGCGCAGGCCCGCGCGCTCT | |
| | ACACCCGGCACCTGCTGCGCCTGAC | |
| | CGCCGACACCCGCGCCGCCACCGTG | |
| | CCGCTCGATCCCGTCCTCAACCCGC | |
| | AGACGAATGCGCCCACCCCGCTGGG | |
| | CGGCGCCGTGCTGCGCGCCACCTCG | |
| | CCCATGCACATGCAGTACCTGCGGA | |
| | ACATGGGCGTCGGGTCGAGCCTGTC | |
| | GGTGTCGGTGGTGGTCGGCGGCCAG | |
| | CTCTGGGGCCTGATCGCCTGCCACC | |
| | ACCAGACGCCCTACGTGTTGCCGCC | |
| | CGACCTGCGAACCACGCTCGAATAC | |
| | CTGGGCCGCTTGCTGAGCCTGCAAG | |
| | TTCAGGTCAAGGAAGCGGCGGACGT | |
| | GGCGGCCTTTCGCCAGAGCCTGCGG | |
| | GAGCACCACGCGCGGGTGGCCCTCG | |
| | CGGCGGCGCACTCGCTCTCGCCGCA | |
| | CGACACCCTCAGTGACCCGGCGCTT | |
| | GACCTGCTGGGCCTGATGCGGGCCG | |
| | GGGGCCTGATTCTGCGTTTCGAGGG | |
| | CCGCTGGCAGACGTTGGGTGAAGTG | |
| | CCGCCTGCCCCGGCGGTGGACGCGC | |
| | TGCTGGCGTGGCTCGAAACCCAGCC | |
| | GGGCGCCCTGGTCCAGACCGACGCG | |
| | CTAGGCCAACTGTGGCTCGCCGGCG | |
| | CCGATCTCGCCCCCAGCGCAGCGGG | |
| | CCTGCTCGCCATCAGCGTGGGCGAG | |
| | GGCTGGTCGGAGTGCCTCGTCTGGC | |
| | TGCGGCCCGAACTGCGGCTGGAGGT | |
| | CGCCTGGGGGGGGCCACTCCTGACC | |
| | AGGCGAAAGACGACCTCGGGCCGCG | |
| | CCACTCATTCGACACCTACCTCGAA | |
| | GAAAAACGCGGCTACGCCGAGCCCT | |
| | GGCATCCCGGCGAAATCGAGGAGGC | |
| | GCAGGATCTACGTGACACATTGACC | |
| | GGGGCGCTGGGCGAGCTCGAGATCG | |
| | CCACTGGGATGGTGGGGGCCCTCCT | |
| | CTTGCTGCTGGTGGTGGCCCTGGGG | |
| | ATCGGCCTCTTCATGCGAAGGCGCC | |
| | ACATCGTTCGGAAGCGCACGCTGCG | |
| | GAGGCTGCTGCAGGAGAGGGAGCTT | |
| | GTGGAGCCTCTTACACCCAGTGGAG | |
| | AAGCTCCCAACCAAGCTCTCTTGAG | |
| | GATCTTGAAGGAAACTGAATTCAAA | |
| | AAGATCAAAGTGCTGGGCTCCGGTG | |
| | CGTTCGGCACGGTGTATAAGGGACT | |
| | CTGGATCCCAGAAGGTGAGAAAGTT | |
| | AAAATTCCCGTCGCTATCAAGGAAT | |
| | TAAGAGAAGCAACATCTCCGAAAGC | |
| | CAACAAGGAAATCCTCGATGAAGCC | |
| | TACGTGATGGCCAGCGTGGACAACC | |
| | CCCACGTGTGCCGCCTGCTGGGCAT | |
| | CTGCCTCACCTCCACCGTGCAACTC | |
| | ATCACGCAGCTCATGCCCTTCGGCT | |
| | GCCTCCTGGACTATGTCCGGGAACA | |
| | CAAAGACAATATTGGCTCCCAGTAC | |
| | CTGCTCAACTGGTGTGTGCAGATCG | |
| | CAAAGGGCATGAACTACTTGGAGGA | |
| | CCGTCGCTTGGTGCACCGCGACCTG | |
| | GCAGCCAGGAACGTACTGGTGAAAA | |
| | CACCGCAGCATGTCAAGATCACAGA | |
| | TTTTGGGCTGGCCAAACTGCTGGGT | |
| | GCGGAAGAGAAAGAATACCATGCAG | |
| | AAGGAGGCAAAGTGCCTATCAAGTG | |
| | GATGGCATTGGAATCAATTTTACAC | |
| | AGAATCTATACCCACCAGAGTGATG | |
| | TCTGGAGCTACGGGGTGACCGTTTG | |
| | GGAGTTGATGACCTTTGGATCCAAG | |
| | CCATATGACGGAATCCCTGCCAGCG | |
| | AGATCTCCTCCATCCTGGAGAAAGG | |
| | AGAACGCCTCCCTCAGCCACCCATA | |
| | TGTACCATCGATGTCTACATGATCA | |
| | TGGTCAAGTGCTGGATGATAGACGC | |
| | AGATAGTCGCCCAAAGTTCCGTGAG | |
| | TTGATCATCGAATTCTCCAAAATGG | |
| | CCCGAGACCCCCAGCGCTACCTTGT | |
| | CATTCAGGGGGATGAAAGAATGCAT | |
| | TTGCCAAGTCCTACAGACTCCAACT | |
| | TCTACCGTGCCCTGATGGATGAAGA | |
| | AGACATGGACGACGTGGTGGATGCC | |
| | GACGAGTACCTCATCCCACAGCAGG | |
| | GCTTCTTCAGCAGCCCCTCCACGTC | |
| | ACGGACTCCCCTCCTGAGCTCTCTG | |
| | AGTGCAACCAGCAACAATTCCACCG | |
| | TGGCTTGCATTGATAGAAATGGGCT | |
| | GCAAAGCTGTCCCATCAAGGAAGAC | |
| | AGCTTCTTGCAGCGATACAGCTCAG | |
| | ACCCCACAGGCGCCTTGACTGAGGA | |
| | CAGCATAGACGACACCTTCCTCCCA | |
| | GTGCCTGAATACATAAACCAGTCCG | |
| | TTCCCAAAAGGCCCGCTGGCTCTGT | |
| | GCAGAATCCTGTCTATCACAATCAG | |
| | CCTCTGAACCCCGCGCCCAGCAGAG | |
| | ACCCACACTACCAGGACCCCCACAG | |
| | CACTGCAGTGGGCAACCCCGAGTAT | |
| | CTCAACACTGTCCAGCCCACCTGTG | |
| | TCAACAGCACATTCGACAGCCCTGC | |
| | CCACTGGGCCCAGAAAGGCAGCCAC | |
| | CAAATTAGCCTGGACAACCCTGACT | |
| | ACCAGCAGGACTTCTTTCCCAAGGA | |
| | AGCCAAGCCAAATGGCATCTTTAAG | |
| | GGCTCCACAGCTGAAAATGCAGAAT | |
| | ACCTAAGGGTCGCGCCACAAAGCAG | |
| | TGAATTTATTGGAGCATCTAGAGGC | |
| | GGTGGTGGCAGCGGAGGAGGGGGGA | |
| | GCGGCGGCGGTGGCAGTGGCGGCGG | |
| | CGGTAGTCGCAGCCGCTTTGTGAAA | |
| | AAAGATAGCGCCGGCAGTGCGGGTA | |
| | GTGCGGGGAGCGCCGGTAGCTATCT | |
| | GGCGAACGAAATTCTGTGGGGCAGC | |
| | GCTGGTAGCGCAGGAAGCGCCGGCA | |
| | GTGCAGGGTTTTGCTATGAAAACGA | |
| | AGTGGCGCTGTCATAATCAGCCATA | |
| | CCACATTTG | |
| 46 | ATGGAGACAGACACACTCCTGCTAT | Fusion |
| | GGGTACTGCTGCTCTGGGTTCCAGG | construct |
| | TTCCACTGGTGACAGCAAGGGCGAG | (HER2) |
| | GAGGATAACATGGCCATCATCAAGG | |
| | AGTTCATGCGCTTCAAGGTGCACAT | |
| | GGAGGGCTCCGTGAACGGCCACGAG | |
| | TTCGAGATCGAGGGCGAGGGCGAGG | |
| | GCCGCCCCTACGAGGGCACCCAGAC | |
| | CGCCAAGCTGAAGGTGACCAAGGGT | |
| | GGCCCCCTGCCCTTCGCCTGGGACA | |
| | TCCTGTCCCCTCAGTTCATGTACGG | |
| | CTCCAAGGCCTACGTGAAGCACCCC | |
| | GCCGACATCCCCGACTACTTGAAGC | |
| | TGTCCTTCCCCGAGGGCTTCAAGTG | |
| | GGAGCGCGTGATGAACTTCGAGGAC | |
| | GGCGGCGTGGTGACCGTGACCCAGG | |
| | ACTCCTCCCTGCAGGACGGCGAGTT | |
| | CATCTACAAGGTGAAGCTGCGCGGC | |
| | ACCAACTTCCCCTCCGACGGCCCCG | |
| | TAATGCAGAAGAAGACCATGGGCTG | |
| | GGAGGCCTCCTCCGAGCGGATGTAC | |
| | CCCGAGGACGGCGCCCTGAAGGGCG | |
| | AGATCAAGCAGAGGCTGAAGCTGAA | |
| | GGACGGCGGCCACTACGACGCTGAG | |
| | GTCAAGACCACCTACAAGGCCAAGA | |
| | AGCCCGTGCAGCTGCCCGGCGCCTA | |
| | CAACGTCAACATCAAGTTGGACATC | |
| | ACCTCCCACAACGAGGACTACACCA | |
| | TCGTGGAACAGTACGAACGCGCCGA | |
| | GGGCCGCCACTCCACCGGCGGCATG | |
| | GACGAGCTGTACAAGGAATTCAGTG | |
| | CTGGTAGTGCTGGTAGTGCTGGCAC | |
| | CGGTATGAGCCGGGACCCGTTGCCC | |
| | TTTTTTCCACCGCTTTACCTTGGTG | |
| | GCCCGGAAATTACCACCGAGAACTG | |
| | CGAGCGCGAGCCGATTCATATTCCC | |
| | GGCAGCATCCAGCCGCACGGCGCCC | |
| | TGCTCACTGCCGACGGGCACAGCGG | |
| | CGAGGTGCTCCAGATGAGCCTCAAC | |
| | GCGGCCACTTTTTTGGGACAGGAAC | |
| | CCACAGTGCTGCGCGGACAGACCCT | |
| | CGCCGCACTGCTGCCCGAGCAGTGG | |
| | CCCGCGCTGCAAGCGGCCCTGCCCC | |
| | CCGGCTGCCCCGACGCCCTGCAATA | |
| | CCGCGCAACGCTGGACTGGCCTGCC | |
| | GCCGGGCACCTTTCGCTGACGGTGC | |
| | ACCGGGTCGGCGAGTTGCTGATTCT | |
| | GGAGTTCGAGCCGACGGAGGCCTGG | |
| | GACAGCACCGGGCCGCACGCGCTGC | |
| | GCAACGCGATGTTCGCGTTCGAAAG | |
| | TGCCCCCAACCTGCGGGCGCTGGCC | |
| | GAGGTGGCGACCCAGACGGTCCGCG | |
| | AGCTGACGGGCTTTGACCGGGTGAT | |
| | GCTCTACAAATTTGCCCCCGACGCC | |
| | ACCGGCGAAGTGATTGCCGAGGCCC | |
| | GCCGTGAGGGGCTGCACGCCTTTCT | |
| | GGGCCACCGTTTTCCCGCGTCGGAC | |
| | ATTCCGGCGCAGGCCCGCGCGCTCT | |
| | ACACCCGGCACCTGCTGCGCCTGAC | |
| | CGCCGACACCCGCGCCGCCACCGTG | |
| | CCGCTCGATCCCGTCCTCAACCCGC | |
| | AGACGAATGCGCCCACCCCGCTGGG | |
| | CGGCGCCGTGCTGCGCGCCACCTCG | |
| | CCCATGCACATGCAGTACCTGCGGA | |
| | ACATGGGCGTCGGGTCGAGCCTGTC | |
| | GGTGTCGGTGGTGGTCGGCGGCCAG | |
| | CTCTGGGGCCTGATCGCCTGCCACC | |
| | ACCAGACGCCCTACGTGTTGCCGCC | |
| | CGACCTGCGAACCACGCTCGAATAC | |
| | CTGGGCCGCTTGCTGAGCCTGCAAG | |
| | TTCAGGTCAAGGAAGCGGCGGACGT | |
| | GGCGGCCTTTCGCCAGAGCCTGCGG | |
| | GAGCACCACGCGCGGGTGGCCCTCG | |
| | CGGCGGCGCACTCGCTCTCGCCGCA | |
| | CGACACCCTCAGTGACCCGGCGCTT | |
| | GACCTGCTGGGCCTGATGCGGGCCG | |
| | GGGGCCTGATTCTGCGTTTCGAGGG | |
| | CCGCTGGCAGACGTTGGGTGAAGTG | |
| | CCGCCTGCCCCGGCGGTGGACGCGC | |
| | TGCTGGCGTGGCTCGAAACCCAGCC | |
| | GGGCGCCCTGGTCCAGACCGACGCG | |
| | CTAGGCCAACTGTGGCTCGCCGGCG | |
| | CCGATCTCGCCCCCAGCGCAGCGGG | |
| | CCTGCTCGCCATCAGCGTGGGCGAG | |
| | GGCTGGTCGGAGTGCCTCGTCTGGC | |
| | TGCGGCCCGAACTGCGGCTGGAGGT | |
| | CGCCTGGGGGGGGGCCACTCCTGAC | |
| | CAGGCGAAAGACGACCTCGGGCCGC | |
| | GCCACTCATTCGACACCTACCTCGA | |
| | AGAAAAACGCGGCTACGCCGAGCCC | |
| | TGGCATCCCGGCGAAATCGAGGAGG | |
| | CGCAGGATCTACGTGACACATTGAC | |
| | CGGGGCGCTGGGCGAGCTCGAGTCC | |
| | ATCATCTCTGCGGTGGTTGGCATTC | |
| | TGCTGGTCGTGGTCTTGGGGGTGGT | |
| | CTTTGGGATCCTCATCAAGCGACGG | |
| | CAGCAGAAGATCCGGAAGTACACGA | |
| | TGCGGAGACTGCTGCAGGAAACGGA | |
| | GCTGGTGGAGCCGCTGACACCTAGC | |
| | GGAGCGATGCCCAACCAGGCGCAGA | |
| | TGCGGATCCTGAAAGAGACGGAGCT | |
| | GAGGAAGGTGAAGGTGCTTGGATCT | |
| | GGCGCTTTTGGCACAGTCTACAAGG | |
| | GCATCTGGATCCCTGATGGGGAGAA | |
| | TGTGAAAATTCCAGTGGCCATCAAA | |
| | GTGTTGAGGGAAAACACATCCCCCA | |
| | AAGCCAACAAAGAAATCTTAGACGA | |
| | AGCATACGTGATGGCTGGTGTGGGC | |
| | TCCCCATATGTCTCCCGCCTTCTGG | |
| | GCATCTGCCTGACATCCACGGTGCA | |
| | GCTGGTGACACAGCTTATGCCCTAT | |
| | GGCTGCCTCTTAGACCATGTCCGGG | |
| | AAAACCGCGGACGCCTGGGCTCCCA | |
| | GGACCTGCTGAACTGGTGTATGCAG | |
| | ATTGCCAAGGGGATGAGCTACCTGG | |
| | AGGATGTGCGGCTCGTACACAGGGA | |
| | CTTGGCCGCTCGGAACGTGCTGGTC | |
| | AAGAGTCCCAACCATGTCAAAATTA | |
| | CAGACTTCGGGCTGGCTCGGCTGCT | |
| | GGACATTGACGAGACAGAGTACCAT | |
| | GCAGATGGGGGCAAGGTGCCCATCA | |
| | AGTGGATGGCGCTGGAGTCCATTCT | |
| | CCGCCGGCGGTTCACCCACCAGAGT | |
| | GATGTGTGGAGTTATGGTGTGACTG | |
| | TGTGGGAGCTGATGACTTTTGGGGC | |
| | CAAACCTTACGATGGGATCCCAGCC | |
| | CGGGAGATCCCTGACCTGCTGGAAA | |
| | AGGGGGAGCGGCTGCCCCAGCCCCC | |
| | CATCTGCACCATTGATGTCTACATG | |
| | ATCATGGTCAAATGTTGGATGATTG | |
| | ACTCTGAATGTCGGCCAAGATTCCG | |
| | GGAGTTGGTGTCTGAATTCTCCCGC | |
| | ATGGCCAGGGACCCCCAGCGCTTTG | |
| | TGGTCATCCAGAATGAGGACTTGGG | |
| | CCCAGCCAGTCCCTTGGACAGCACC | |
| | TTCTACCGCTCACTGCTGGAGGACG | |
| | ATGACATGGGGGACCTGGTGGATGC | |
| | TGAGGAGTATCTGGTACCCCAGCAG | |
| | GGCTTCTTCTGTCCAGACCCTGCCC | |
| | CGGGCGCTGGGGGCATGGTCCACCA | |
| | CAGGCACCGCAGCTCATCTACCAGG | |
| | AGTGGCGGTGGGGACCTGACACTAG | |
| | GGCTGGAGCCCTCTGAAGAGGAGGC | |
| | CCCCAGGTCTCCACTGGCACCCTCC | |
| | GAAGGGGCTGGCTCCGATGTATTTG | |
| | ATGGTGACCTGGGAATGGGGGCAGC | |
| | CAAGGGGCTGCAAAGCCTCCCCACA | |
| | CATGACCCCAGCCCTCTACAGCGGT | |
| | ACAGTGAGGACCCCACAGTACCCCT | |
| | GCCCTCTGAGACTGATGGCTACGTT | |
| | GCCCCCCTGACCTGCAGCCCCCAGC | |
| | CTGAATATGTGAACCAGCCAGATGT | |
| | TCGGCCCCAGCCCCCTTCGCCCCGA | |
| | GAGGGCCCTCTGCCTGCTGCCCGAC | |
| | CTGCTGGTGCCACTCTGGAAAGGCC | |
| | CAAGACTCTCTCCCCAGGGAAGAAT | |
| | GGGGTCGTCAAAGACGTTTTTGCCT | |
| | TTGGGGGTGCCGTGGAGAACCCCGA | |
| | GTACTTGACACCCCAGGGAGGAGCT | |
| | GCCCCTCAGCCCCACCCTCCTCCTG | |
| | CCTTCAGCCCAGCCTTCGACAACCT | |
| | CTATTACTGGGACCAGGACCCACCA | |
| | GAGCGGGGGGCTCCACCCAGCACCT | |
| | TCAAAGGGACACCTACGGCAGAGAA | |
| | CCCAGAGTACCTGGGTCTGGACGTG | |
| | CCAGTGTCTAGAGGCGGTGGTGGCA | |
| | GCGGAGGAGGGGGGAGCGGCGGCGG | |
| | TGGCAGTGGCGGCGGCGGTAGTCGC | |
| | AGCCGCTTTGTGAAAAAAGATAGCG | |
| | CCGGCAGTGCGGGTAGTGCGGGGAG | |
| | CGCCGGTAGCTATCTGGCGAACGAA | |
| | ATTCTGTGGGGCAGCGCTGGTAGCG | |
| | CAGGAAGCGCCGGCAGTGCAGGGTT | |
| | TTGCTATGAAAACGAAGTGGCGCTG | |
| | TCATAATCAGCCATACCACATTTG | |
| 47 | ATGGAGACAGACACACTCCTGCTA | Fusion |
| | TGGGTACTGCTGCTCTGGGTTCCAG | construct |
| | GTTCCACTGGTGACAGCAAGGGCGA | (FGFR1) |
| | GGAGGATAACATGGCCATCATCAAG | |
| | GAGTTCATGCGCTTCAAGGTGCACA | |
| | TGGAGGGCTCCGTGAACGGCCACGA | |
| | GTTCGAGATCGAGGGCGAGGGCGAG | |
| | GGCCGCCCCTACGAGGGCACCCAGA | |
| | CCGCCAAGCTGAAGGTGACCAAGGG | |
| | TGGCCCCCTGCCCTTCGCCTGGGAC | |
| | ATCCTGTCCCCTCAGTTCATGTACG | |
| | GCTCCAAGGCCTACGTGAAGCACCC | |
| | CGCCGACATCCCCGACTACTTGAAG | |
| | CTGTCCTTCCCCGAGGGCTTCAAGT | |
| | GGGAGCGCGTGATGAACTTCGAGGA | |
| | CGGCGGCGTGGTGACCGTGACCCAG | |
| | GACTCCTCCCTGCAGGACGGCGAGT | |
| | TCATCTACAAGGTGAAGCTGCGCGG | |
| | CACCAACTTCCCCTCCGACGGCCCC | |
| | GTAATGCAGAAGAAGACCATGGGCT | |
| | GGGAGGCCTCCTCCGAGCGGATGTA | |
| | CCCCGAGGACGGCGCCCTGAAGGGC | |
| | GAGATCAAGCAGAGGCTGAAGCTGA | |
| | AGGACGGCGGCCACTACGACGCTGA | |
| | GGTCAAGACCACCTACAAGGCCAAG | |
| | AAGCCCGTGCAGCTGCCCGGCGCCT | |
| | ACAACGTCAACATCAAGTTGGACAT | |
| | CACCTCCCACAACGAGGACTACACC | |
| | ATCGTGGAACAGTACGAACGCGCCG | |
| | AGGGCCGCCACTCCACCGGCGGCAT | |
| | GGACGAGCTGTACAAGGAATTCAGT | |
| | GCTGGTAGTGCTGGTAGTGCTGGCA | |
| | CCGGTATGAGCCGGGACCCGTTGCC | |
| | CTTTTTTCCACCGCTTTACCTTGGT | |
| | GGCCCGGAAATTACCACCGAGAACT | |
| | GCGAGCGCGAGCCGATTCATATTCC | |
| | CGGCAGCATCCAGCCGCACGGCGCC | |
| | CTGCTCACTGCCGACGGGCACAGCG | |
| | GCGAGGTGCTCCAGATGAGCCTCAA | |
| | CGCGGCCACTTTTTTGGGACAGGAA | |
| | CCCACAGTGCTGCGCGGACAGACCC | |
| | TCGCCGCACTGCTGCCCGAGCAGTG | |
| | GCCCGCGCTGCAAGCGGCCCTGCCC | |
| | CCCGGCTGCCCCGACGCCCTGCAAT | |
| | ACCGCGCAACGCTGGACTGGCCTGC | |
| | CGCCGGGCACCTTTCGCTGACGGTG | |
| | CACCGGGTCGGCGAGTTGCTGATTC | |
| | TGGAGTTCGAGCCGACGGAGGCCTG | |
| | GGACAGCACCGGGCCGCACGCGCTG | |
| | CGCAACGCGATGTTCGCGTTCGAAA | |
| | GTGCCCCCAACCTGCGGGCGCTGGC | |
| | CGAGGTGGCGACCCAGACGGTCCGC | |
| | GAGCTGACGGGCTTTGACCGGGTGA | |
| | TGCTCTACAAATTTGCCCCCGACGC | |
| | CACCGGCGAAGTGATTGCCGAGGCC | |
| | CGCCGTGAGGGGCTGCACGCCTTTC | |
| | TGGGCCACCGTTTTCCCGCGTCGGA | |
| | CATTCCGGCGCAGGCCCGCGCGCTC | |
| | TACACCCGGCACCTGCTGCGCCTGA | |
| | CCGCCGACACCCGCGCCGCCACCGT | |
| | GCCGCTCGATCCCGTCCTCAACCCG | |
| | CAGACGAATGCGCCCACCCCGCTGG | |
| | GCGGCGCCGTGCTGCGCGCCACCTC | |
| | GCCCATGCACATGCAGTACCTGCGG | |
| | AACATGGGCGTCGGGTCGAGCCTGT | |
| | CGGTGTCGGTGGTGGTCGGCGGCCA | |
| | GCTCTGGGGCCTGATCGCCTGCCAC | |
| | CACCAGACGCCCTACGTGTTGCCGC | |
| | CCGACCTGCGAACCACGCTCGAATA | |
| | CCTGGGCCGCTTGCTGAGCCTGCAA | |
| | GTTCAGGTCAAGGAAGCGGCGGACG | |
| | TGGCGGCCTTTCGCCAGAGCCTGCG | |
| | GGAGCACCACGCGCGGGTGGCCCTC | |
| | GCGGCGGCGCACTCGCTCTCGCCGC | |
| | ACGACACCCTCAGTGACCCGGCGCT | |
| | TGACCTGCTGGGCCTGATGCGGGCC | |
| | GGGGGCCTGATTCTGCGTTTCGAGG | |
| | GCCGCTGGCAGACGTTGGGTGAAGT | |
| | GCCGCCTGCCCCGGCGGTGGACGCG | |
| | CTGCTGGCGTGGCTCGAAACCCAGC | |
| | CGGGCGCCCTGGTCCAGACCGACGC | |
| | GCTAGGCCAACTGTGGCTCGCCGGC | |
| | GCCGATCTCGCCCCCAGCGCAGCGG | |
| | GCCTGCTCGCCATCAGCGTGGGCGA | |
| | GGGCTGGTCGGAGTGCCTCGTCTGG | |
| | CTGCGGCCCGAACTGCGGCTGGAGG | |
| | TCGCCTGGGGGGGGGCCACTCCTGA | |
| | CCAGGCGAAAGACGACCTCGGGCCG | |
| | CGCCACTCATTCGACACCTACCTCG | |
| | AAGAAAAACGCGGCTACGCCGAGCC | |
| | CTGGCATCCCGGCGAAATCGAGGAG | |
| | GCGCAGGATCTACGTGACACATTGA | |
| | CCGGGGCGCTGGGCGAGTACTTTTA | |
| | TTTCTCCATCGTCTCTGCGGTGGTT | |
| | GGCATTCTGCTGGTCGTGGTCTTGG | |
| | GGGTGGTCTTTGGGTCTAGAAAGAT | |
| | GAAGAGTGGTACCAAGAAGAGTGAC | |
| | TTCCACAGCCAGATGGCTGTGCACA | |
| | AGCTGGCCAAGAGCATCCCTCTGCG | |
| | CAGACAGGTAACAGTGTCTGCTGAC | |
| | TCCAGTGCATCCATGAACTCTGGGG | |
| | TTCTTCTGGTTCGGCCATCACGGCT | |
| | CTCCTCCAGTGGGACTCCCATGCTA | |
| | GCAGGGGTCTCTGAGTATGAGCTTC | |
| | CCGAAGACCTTCGCTGGGAGCTGCC | |
| | TCGGGACAGACTGGTCTTAGGCAAA | |
| | CCCCTGGGAGAGGGCTGCTTTGGGC | |
| | AGGTGGTGTTGGCAGAGGCTATCGG | |
| | GCTGGACAAGGACAAACCCAACCGT | |
| | GTGACCAAAGTGGCTGTGAAGATGT | |
| | TGAAGTCGGACGCAACAGAGAAAGA | |
| | CTTGTCAGACCTGATCTCAGAAATG | |
| | GAGATGATGAAGATGATCGGGAAGC | |
| | ATAAGAATATCATCAACCTGCTGGG | |
| | GGCCTGCACGCAGGATGGTCCCTTG | |
| | TATGTCATCGTGGAGTATGCCTCCA | |
| | AGGGCAACCTGCGGGAGTACCTGCA | |
| | GGCCCGGAGGCCCCCAGGGCTGGAA | |
| | TACTGCTACAACCCCAGCCACAACC | |
| | CAGAGGAGCAGCTCTCCTCCAAGGA | |
| | CCTGGTGTCCTGCGCCTACCAGGTG | |
| | GCCCGAGGCATGGAGTATCTGGCCT | |
| | CCAAGAAGTGCATACACCGAGACCT | |
| | GGCAGCCAGGAATGTCCTGGTGACA | |
| | GAGGACAATGTGATGAAGATAGCAG | |
| | ACTTTGGCCTCGCACGGGACATTCA | |
| | CCACATCGACTACTATAAAAAGACA | |
| | ACCAACGGCCGACTGCCTGTGAAGT | |
| | GGATGGCACCCGAGGCATTATTTGA | |
| | CCGGATCTACACCCACCAGAGTGAT | |
| | GTGTGGTCTTTCGGGGTGCTCCTGT | |
| | GGGAGATCTTCACTCTGGGCGGCTC | |
| | CCCATACCCCGGTGTGCCTGTGGAG | |
| | GAACTTTTCAAGCTGCTGAAGGAGG | |
| | GTCACCGCATGGACAAGCCCAGTAA | |
| | CTGCACCAACGAGCTGTACATGATG | |
| | ATGCGGGACTGCTGGCATGCAGTGC | |
| | CCTCACAGAGACCCACCTTCAAGCA | |
| | GCTGGTGGAAGACCTGGACCGCATC | |
| | GTGGCCTTGACCTCCAACCAGGAGT | |
| | ACCTGGACCTGTCCATGCCCCTGGA | |
| | CCAGTACTCCCCCAGCTTTCCCGAC | |
| | ACCCGGAGCTCTACGTGCTCCTCAG | |
| | GGGAGGATTCCGTCTTCTCTCATGA | |
| | GCCGCTGCCCGAGGAGCCCTGCCTG | |
| | CCCCGACACCCAGCCCAGCTTGCCA | |
| | ATGGCGGACTCAAACGCCGCTCTAG | |
| | AGGCGGTGGTGGCAGCGGAGGAGGG | |
| | GGGAGCGGCGGCGGTGGCAGTGGCG | |
| | GCGGCGGTAGTCGCAGCCGCTTTGT | |
| | GAAAAAAGATAGCGCCGGCAGTGCG | |
| | GGTAGTGCGGGGAGCGCCGGTAGCT | |
| | ATCTGGCGAACGAAATTCTGTGGGG | |
| | CAGCGCTGGTAGCGCAGGAAGCGCC | |
| | GGCAGTGCAGGGTTTTGCTATGAAA | |
| | ACGAAGTGGCGCTGTCATAATCAGC | |
| | CATACCACATTTG | |
| 48 | ATGGAGACAGACACACTCCTGCTAT | Fusion |
| | GGGTACTGCTGCTCTGGGTTCCAGG | construct |
| | TTCCACTGGTGACAGCAAGGGCGAG | (TrkA) |
| | GAGGATAACATGGCCATCATCAAGG | |
| | AGTTCATGCGCTTCAAGGTGCACAT | |
| | GGAGGGCTCCGTGAACGGCCACGAG | |
| | TTCGAGATCGAGGGCGAGGGCGAGG | |
| | GCCGCCCCTACGAGGGCACCCAGAC | |
| | CGCCAAGCTGAAGGTGACCAAGGGT | |
| | GGCCCCCTGCCCTTCGCCTGGGACA | |
| | TCCTGTCCCCTCAGTTCATGTACGG | |
| | CTCCAAGGCCTACGTGAAGCACCCC | |
| | GCCGACATCCCCGACTACTTGAAGC | |
| | TGTCCTTCCCCGAGGGCTTCAAGTG | |
| | GGAGCGCGTGATGAACTTCGAGGAC | |
| | GGCGGCGTGGTGACCGTGACCCAGG | |
| | ACTCCTCCCTGCAGGACGGCGAGTT | |
| | CATCTACAAGGTGAAGCTGCGCGGC | |
| | ACCAACTTCCCCTCCGACGGCCCCG | |
| | TAATGCAGAAGAAGACCATGGGCTG | |
| | GGAGGCCTCCTCCGAGCGGATGTAC | |
| | CCCGAGGACGGCGCCCTGAAGGGCG | |
| | AGATCAAGCAGAGGCTGAAGCTGAA | |
| | GGACGGCGGCCACTACGACGCTGAG | |
| | GTCAAGACCACCTACAAGGCCAAGA | |
| | AGCCCGTGCAGCTGCCCGGCGCCTA | |
| | CAACGTCAACATCAAGTTGGACATC | |
| | ACCTCCCACAACGAGGACTACACCA | |
| | TCGTGGAACAGTACGAACGCGCCGA | |
| | GGGCCGCCACTCCACCGGCGGCATG | |
| | GACGAGCTGTACAAGGAATTCAGTG | |
| | CTGGTAGTGCTGGTAGTGCTGGCAC | |
| | CGGTATGAGCCGGGACCCGTTGCCC | |
| | TTTTTTCCACCGCTTTACCTTGGTG | |
| | GCCCGGAAATTACCACCGAGAACTG | |
| | CGAGCGCGAGCCGATTCATATTCCC | |
| | GGCAGCATCCAGCCGCACGGCGCCC | |
| | TGCTCACTGCCGACGGGCACAGCGG | |
| | CGAGGTGCTCCAGATGAGCCTCAAC | |
| | GCGGCCACTTTTTTGGGACAGGAAC | |
| | CCACAGTGCTGCGCGGACAGACCCT | |
| | CGCCGCACTGCTGCCCGAGCAGTGG | |
| | CCCGCGCTGCAAGCGGCCCTGCCCC | |
| | CCGGCTGCCCCGACGCCCTGCAATA | |
| | CCGCGCAACGCTGGACTGGCCTGCC | |
| | GCCGGGCACCTTTCGCTGACGGTGC | |
| | ACCGGGTCGGCGAGTTGCTGATTCT | |
| | GGAGTTCGAGCCGACGGAGGCCTGG | |
| | GACAGCACCGGGCCGCACGCGCTGC | |
| | GCAACGCGATGTTCGCGTTCGAAAG | |
| | TGCCCCCAACCTGCGGGCGCTGGCC | |
| | GAGGTGGCGACCCAGACGGTCCGCG | |
| | AGCTGACGGGCTTTGACCGGGTGAT | |
| | GCTCTACAAATTTGCCCCCGACGCC | |
| | ACCGGCGAAGTGATTGCCGAGGCCC | |
| | GCCGTGAGGGGCTGCACGCCTTTCT | |
| | GGGCCACCGTTTTCCCGCGTCGGAC | |
| | ATTCCGGCGCAGGCCCGCGCGCTCT | |
| | ACACCCGGCACCTGCTGCGCCTGAC | |
| | CGCCGACACCCGCGCCGCCACCGTG | |
| | CCGCTCGATCCCGTCCTCAACCCGC | |
| | AGACGAATGCGCCCACCCCGCTGGG | |
| | CGGCGCCGTGCTGCGCGCCACCTCG | |
| | CCCATGCACATGCAGTACCTGCGGA | |
| | ACATGGGCGTCGGGTCGAGCCTGTC | |
| | GGTGTCGGTGGTGGTCGGCGGCCAG | |
| | CTCTGGGGCCTGATCGCCTGCCACC | |
| | ACCAGACGCCCTACGTGTTGCCGCC | |
| | CGACCTGCGAACCACGCTCGAATAC | |
| | CTGGGCCGCTTGCTGAGCCTGCAAG | |
| | TTCAGGTCAAGGAAGCGGCGGACGT | |
| | GGCGGCCTTTCGCCAGAGCCTGCGG | |
| | GAGCACCACGCGCGGGTGGCCCTCG | |
| | CGGCGGCGCACTCGCTCTCGCCGCA | |
| | CGACACCCTCAGTGACCCGGCGCTT | |
| | GACCTGCTGGGCCTGATGCGGGCCG | |
| | GGGGCCTGATTCTGCGTTTCGAGGG | |
| | CCGCTGGCAGACGTTGGGTGAAGTG | |
| | CCGCCTGCCCCGGCGGTGGACGCGC | |
| | TGCTGGCGTGGCTCGAAACCCAGCC | |
| | GGGCGCCCTGGTCCAGACCGACGCG | |
| | CTAGGCCAACTGTGGCTCGCCGGCG | |
| | CCGATCTCGCCCCCAGCGCAGCGGG | |
| | CCTGCTCGCCATCAGCGTGGGCGAG | |
| | GGCTGGTCGGAGTGCCTCGTCTGGC | |
| | TGCGGCCCGAACTGCGGCTGGAGGT | |
| | CGCCTGGGGGGGGGCCACTCCTGAC | |
| | CAGGCGAAAGACGACCTCGGGCCGC | |
| | GCCACTCATTCGACACCTACCTCGA | |
| | AGAAAAACGCGGCTACGCCGAGCCC | |
| | TGGCATCCCGGCGAAATCGAGGAGG | |
| | CGCAGGATCTACGTGACACATTGAC | |
| | CGGGGCGCTGGGCGAGTACTTTTAT | |
| | TTCTCCATCGTCTCTGCGGTGGTTG | |
| | GCATTCTGCTGGTCGTGGTCTTGGG | |
| | GGTGGTCTTTGGGTCTAGAAACAAA | |
| | TGTGGACGGAGAAACAAGTTTGGGA | |
| | TCAACCGCCCGGCTGTGCTGGCTCC | |
| | AGAGGATGGGCTGGCCATGTCCCTG | |
| | CATTTCATGACATTGGGTGGCAGCT | |
| | CCCTGTCCCCCACCGAGGGCAAAGG | |
| | CTCTGGGCTCCAAGGCCACATCATC | |
| | GAGAACCCACAATACTTCAGTGATG | |
| | CCTGTGTTCACCACATCAAGCGCCG | |
| | GGACATCGTGCTCAAGTGGGAGCTG | |
| | GGGGAGGGCGCCTTTGGGAAGGTCT | |
| | TCCTTGCTGAGTGCCACAACCTCCT | |
| | GCCTGAGCAGGACAAGATGCTGGTG | |
| | GCTGTCAAGGCACTGAAGGAGGCGT | |
| | CCGAGAGTGCTCGGCAGGACTTCCA | |
| | ACGTGAGGCTGAGCTGCTCACCATG | |
| | CTGCAGCACCAGCACATCGTGCGCT | |
| | TCTTCGGCGTCTGCACCGAGGGCCG | |
| | CCCCCTGCTCATGGTCTTCGAGTAT | |
| | ATGCGGCACGGGGACCTCAACCGCT | |
| | TCCTCCGATCCCATGGACCCGATGC | |
| | CAAGCTGCTGGCTGGTGGGGAGGAT | |
| | GTGGCTCCAGGCCCCCTGGGTCTGG | |
| | GGCAGCTGCTGGCCGTGGCTAGCCA | |
| | GGTCGCTGCGGGGATGGTGTACCTG | |
| | GCGGGTCTGCATTTTGTGCACCGGG | |
| | ACCTGGCCACACGCAACTGTCTAGT | |
| | GGGCCAGGGACTGGTGGTCAAGATT | |
| | GGTGATTTTGGCATGAGCAGGGATA | |
| | TCTACAGCACCGACTATTACCGTGT | |
| | GGGAGGCCGCACCATGCTGCCCATT | |
| | CGCTGGATGCCGCCCGAGAGCATCC | |
| | TGTACCGTAAGTTCACCACCGAGAG | |
| | CGACGTGTGGAGCTTCGGCGTGGTG | |
| | CTCTGGGAGATCTTCACCTACGGCA | |
| | AGCAGCCCTGGTACCAGCTCTCCAA | |
| | CACGGAGGCAATCGACTGCATCACG | |
| | CAGGGACGTGAGTTGGAGCGGCCAC | |
| | GTGCCTGCCCACCAGAGGTCTACGC | |
| | CATCATGCGGGGCTGCTGGCAGCGG | |
| | GAGCCCCAGCAACGCCACAGCATCA | |
| | AGGATGTGCACGCCCGGCTGCAAGC | |
| | CCTGGCCCAGGCACCTCCTGTCTAC | |
| | CTGGATGTCCTGGGCTCTAGAGGCG | |
| | GTGGTGGCAGCGGAGGAGGGGGGAG | |
| | CGGCGGCGGTGGCAGTGGCGGCGGC | |
| | GGTAGTCGCAGCCGCTTTGTGAAAA | |
| | AAGATAGCGCCGGCAGTGCGGGTAG | |
| | TGCGGGGAGCGCCGGTAGCTATCTG | |
| | GCGAACGAAATTCTGTGGGGCAGCG | |
| | CTGGTAGCGCAGGAAGCGCCGGCAG | |
| | TGCAGGGTTTTGCTATGAAAACGAA | |
| | GTGGCGCTGTCATAATCAGCCATAC | |
| | CACATTTG | |
| 49 | ATGGAGACAGACACACTCCTGCTAT | Fusion |
| | GGGTACTGCTGCTCTGGGTTCCAGG | construct |
| | TTCCACTGGTGACAGCAAGGGCGAG | (TrkB) |
| | GAGGATAACATGGCCATCATCAAGG | |
| | AGTTCATGCGCTTCAAGGTGCACAT | |
| | GGAGGGCTCCGTGAACGGCCACGAG | |
| | TTCGAGATCGAGGGCGAGGGCGAGG | |
| | GCCGCCCCTACGAGGGCACCCAGAC | |
| | CGCCAAGCTGAAGGTGACCAAGGGT | |
| | GGCCCCCTGCCCTTCGCCTGGGACA | |
| | TCCTGTCCCCTCAGTTCATGTACGG | |
| | CTCCAAGGCCTACGTGAAGCACCCC | |
| | GCCGACATCCCCGACTACTTGAAGC | |
| | TGTCCTTCCCCGAGGGCTTCAAGTG | |
| | GGAGCGCGTGATGAACTTCGAGGAC | |
| | GGCGGCGTGGTGACCGTGACCCAGG | |
| | ACTCCTCCCTGCAGGACGGCGAGTT | |
| | CATCTACAAGGTGAAGCTGCGCGGC | |
| | ACCAACTTCCCCTCCGACGGCCCCG | |
| | TAATGCAGAAGAAGACCATGGGCTG | |
| | GGAGGCCTCCTCCGAGCGGATGTAC | |
| | CCCGAGGACGGCGCCCTGAAGGGCG | |
| | AGATCAAGCAGAGGCTGAAGCTGAA | |
| | GGACGGCGGCCACTACGACGCTGAG | |
| | GTCAAGACCACCTACAAGGCCAAGA | |
| | AGCCCGTGCAGCTGCCCGGCGCCTA | |
| | CAACGTCAACATCAAGTTGGACATC | |
| | ACCTCCCACAACGAGGACTACACCA | |
| | TCGTGGAACAGTACGAACGCGCCGA | |
| | GGGCCGCCACTCCACCGGCGGCATG | |
| | GACGAGCTGTACAAGGAATTCAGTG | |
| | CTGGTAGTGCTGGTAGTGCTGGCAC | |
| | CGGTATGAGCCGGGACCCGTTGCCC | |
| | TTTTTTCCACCGCTTTACCTTGGTG | |
| | GCCCGGAAATTACCACCGAGAACTG | |
| | CGAGCGCGAGCCGATTCATATTCCC | |
| | GGCAGCATCCAGCCGCACGGCGCCC | |
| | TGCTCACTGCCGACGGGCACAGCGG | |
| | CGAGGTGCTCCAGATGAGCCTCAAC | |
| | GCGGCCACTTTTTTGGGACAGGAAC | |
| | CCACAGTGCTGCGCGGACAGACCCT | |
| | CGCCGCACTGCTGCCCGAGCAGTGG | |
| | CCCGCGCTGCAAGCGGCCCTGCCCC | |
| | CCGGCTGCCCCGACGCCCTGCAATA | |
| | CCGCGCAACGCTGGACTGGCCTGCC | |
| | GCCGGGCACCTTTCGCTGACGGTGC | |
| | ACCGGGTCGGCGAGTTGCTGATTCT | |
| | GGAGTTCGAGCCGACGGAGGCCTGG | |
| | GACAGCACCGGGCCGCACGCGCTGC | |
| | GCAACGCGATGTGGGCATGGTCTAC | |
| | CTGGCGTCCCAGCACTTCGTGCACC | |
| | GCGATTTGGCCACCAGGAACTGCCT | |
| | GGTCGGGGAGAACTTGCTGGTGAAA | |
| | ATCGGGGACTTTGGGATGTCCCGGG | |
| | ACGTGTACAGCACTGACTACTACAG | |
| | GGTCGGTGGCCACACAATGCTGCCC | |
| | ATTCGCTGGATGCCTCCAGAGAGCA | |
| | TCATGTACAGGAAATTCACGACGGA | |
| | AAGCGACGTCTGGAGCCTGGGGGTC | |
| | GTGTTGTGGGAGATTTTCACCTATG | |
| | GCAAACAGCCCTGGTACCAGCTGTC | |
| | AAACAATGAGGTGATAGAGTGTATC | |
| | ACTCAGGGCCGAGTCCTGCAGCGAC | |
| | CCCGCACGTGCCCCCAGGAGGTGTA | |
| | TGAGCTGATGCTGGGGTGCTGGCAG | |
| | CGAGAGCCCCACATGAGGAAGAACA | |
| | TCAAGGGCATCCATACCCTCCTTCA | |
| | GAACTTGGCCAAGGCATCTCCGGTC | |
| | TACCTGGACATTCTAGGCTCTAGAG | |
| | GCGGTGGTGGCAGCGGAGGAGGGGG | |
| | GAGCGGCGGCGGTGGCAGTGGCGGC | |
| | GGCGGTAGTCGCAGCCGCTTTGTGA | |
| | AAAAAGATAGCGCCGGCAGTGCGGG | |
| | TAGTGCGGGGAGCGCCGGTAGCTAT | |
| | CTGGCGAACGAAATTCTGTGGGGCA | |
| | GCGCTGGTAGCGCAGGAAGCGCCGG | |
| | CAGTGCAGGGTTTTGCTATGAAAAC | |
| | GAAGTGGCGCTGTCATAATCAGCCA | |
| | TACCACATTTG | |
| 50 | ATGGAGACAGACACACTCCTGCTAT | Fusion |
| | GGGTACTGCTGCTCTGGGTTCCAGG | construct |
| | TTCCACTGGTGACAGCAAGGGCGAG | (cKIT) |
| | GAGGATAACATGGCCATCATCAAGG | |
| | AGTTCATGCGCTTCAAGGTGCACAT | |
| | GGAGGGCTCCGTGAACGGCCACGAG | |
| | TTCGAGATCGAGGGCGAGGGCGAGG | |
| | GCCGCCCCTACGAGGGCACCCAGAC | |
| | CGCCAAGCTGAAGGTGACCAAGGGT | |
| | GGCCCCCTGCCCTTCGCCTGGGACA | |
| | TCCTGTCCCCTCAGTTCATGTACGG | |
| | CTCCAAGGCCTACGTGAAGCACCCC | |
| | GCCGACATCCCCGACTACTTGAAGC | |
| | TGTCCTTCCCCGAGGGCTTCAAGTG | |
| | GGAGCGCGTGATGAACTTCGAGGAC | |
| | GGCGGCGTGGTGACCGTGACCCAGG | |
| | ACTCCTCCCTGCAGGACGGCGAGTT | |
| | CATCTACAAGGTGAAGCTGCGCGGC | |
| | ACCAACTTCCCCTCCGACGGCCCCG | |
| | TAATGCAGAAGAAGACCATGGGCTG | |
| | GGAGGCCTCCTCCGAGCGGATGTAC | |
| | CCCGAGGACGGCGCCCTGAAGGGCG | |
| | AGATCAAGCAGAGGCTGAAGCTGAA | |
| | GGACGGCGGCCACTACGACGCTGAG | |
| | GTCAAGACCACCTACAAGGCCAAGA | |
| | AGCCCGTGCAGCTGCCCGGCGCCTA | |
| | CAACGTCAACATCAAGTTGGACATC | |
| | ACCTCCCACAACGAGGACTACACCA | |
| | TCGTGGAACAGTACGAACGCGCCGA | |
| | GGGCCGCCACTCCACCGGCGGCATG | |
| | GACGAGCTGTACAAGGAATTCAGTG | |
| | CTGGTAGTGCTGGTAGTGCTGGCAC | |
| | CGGTATGAGCCGGGACCCGTTGCCC | |
| | TTTTTTCCACCGCTTTACCTTGGTG | |
| | GCCCGGAAATTACCACCGAGAACTG | |
| | CGAGCGCGAGCCGATTCATATTCCC | |
| | GGCAGCATCCAGCCGCACGGCGCCC | |
| | TGCTCACTGCCGACGGGCACAGCGG | |
| | CGAGGTGCTCCAGATGAGCCTCAAC | |
| | GCGGCCACTTTTTTGGGACAGGAAC | |
| | CCACAGTGCTGCGCGGACAGACCCT | |
| | CGCCGCACTGCTGCCCGATCGCGTT | |
| | CGAAAGTGCCCCCAACCTGCGGGCG | |
| | CTGGCCGAGGTGGCGACCCAGACGG | |
| | TCCGCGAGCTGACGGGCTTTGACCG | |
| | GGTGATGCTCTACAAATTTGCCCCC | |
| | GACGCCACCGGCGAAGTGATTGCCG | |
| | AGGCCCGCCGTGAGGGGCTGCACGC | |
| | CTTTCTGGGCCACCGTTTTCCCGCG | |
| | TCGGACATTCCGGCGCAGGCCCGCG | |
| | CGCTCTACACCCGGCACCTGCTGCG | |
| | CCTGACCGCCGACACCCGCGCCGCC | |
| | ACCGTGCCGCTCGATCCCGTCCTCA | |
| | ACCCGCAGACGAATGCGCCCACCCC | |
| | GCTGGGCGGCGCCGTGCTGCGCGCC | |
| | ACCTCGCCCATGCACATGCAGTACC | |
| | TGCGGAACATGGGCGTCGGGTCGAG | |
| | CCTGTCGGTGTCGGTGGTGGTCGGC | |
| | GGCCAGCTCTGGGGCCTGATCGCCT | |
| | GCCACCACCAGACGCCCTACGTGTT | |
| | GCCGCCCGACCTGCGAACCACGCTC | |
| | GAATACCTGGGCCGCTTGCTGAGCC | |
| | TGCAAGTTCAGGTCAAGGAAGCGGC | |
| | GGACGTGGCGGCCTTTCGCCAGAGC | |
| | CTGCGGGAGCACCACGCGCGGGTGG | |
| | CCCTCGCGGCGGCGCACTCGCTCTC | |
| | GCCGCACGACACCCTCAGTGACCCG | |
| | GCGCTTGACCTGCTGGGCCTGATGC | |
| | GGGCCGGGGGCCTGATTCTGCGTTT | |
| | CGAGGGCCGCTGGCAGACGTTGGGT | |
| | GAAGTGCCGCCTGCCCCGGCGGTGG | |
| | ACGCGCTGCTGGCGTGGCTCGAAAC | |
| | CCAGCCGGGCGCCCTGGTCCAGACC | |
| | GACGCGCTAGGCCAACTGTGGCTCG | |
| | CCGGCGCCGATCTCGCCCCCAGCGC | |
| | AGCGGGCCTGCTCGCCATCAGCGTG | |
| | GGCGAGGGCTGGTCGGAGTGCCTCG | |
| | TCTGGCTGCGGCCCGAACTGCGGCT | |
| | GGAGGTCGCCTGGGGCGGGGCCACT | |
| | CCTGACCAGGCGAAAGACGACCTCG | |
| | GGCCGCGCCACTCATTCGACACCTA | |
| | CCTCGAAGAAAAACGCGGCTACGCC | |
| | GAGCCCTGGCATCCCGGCGAAATCG | |
| | AGGAGGCGCAGGATCTACGTGACAC | |
| | ATTGACCGGGGCGCTGGGCGAGCTC | |
| | GAGATCGCCACTGGGATGGTGGGGG | |
| | CCCTCCTCTTGCTGCTGGTGGTGGC | |
| | CCTGGGGATCGGCCTCTTCATGTCT | |
| | AGAAAGTTGGCAAGACACTCCAAGT | |
| | TTGGCATGAAAGGCCCAGCCTCCGT | |
| | TATCAGCAATGATGATGACTCTGCC | |
| | AGCCCACTCCATCACATCTCCAATG | |
| | GGAGTAACACTCCATCTTCTTCGGA | |
| | AGGTGGCCCAGATGCTGTCATTATT | |
| | GGAATGACCAAGATCCCTGTCATTG | |
| | AAAATCCCCAGTACTTTGGCATCAC | |
| | CAACAGTCAGCTCAAGCCAGACACA | |
| | TTTGTTCAGCACATCAAGCGACATA | |
| | ACATTGTTCTGAAAAGGGAGCTAGG | |
| | CGAAGGAGCCTTTGGAAAAGTGTTC | |
| | CTAGCTGAATGCTATAACCTCTGTC | |
| | CTGAGCAGGACAAGATCTTGGTGGC | |
| | AGTGAAGACCCTGAAGGATGCCAGT | |
| | GACAATGCACGCAAGGACTTCCACC | |
| | GTGAGGCCGAGCTCCTGACCAACCT | |
| | CCAGCATGAGCACATCGTCAAGTTC | |
| | TATGGCGTCTGCGTGGAGGGCGACC | |
| | CCCTCATCATGGTCTTTGAGTACAT | |
| | GAAGCATGGGGACCTCAACAAGTTC | |
| | CTCAGGGCACACGGCCCTGATGCCG | |
| | TGCTGATGGCTGAGGGCAACCCGCC | |
| | CACGGAACTGACGCAGTCGCAGATG | |
| | CTGCATATAGCCCAGCAGATCGCCG | |
| | CGCAGTGGCCCGCGCTGCAAGCGGC | |
| | CCTGCCCCCCGGCTGCCCCGACGCC | |
| | CTGCAATACCGCGCAACGCTGGACT | |
| | GGCCTGCCGCCGGGCACCTTTCGCT | |
| | GACGGTGCACCGGGTCGGCGAGTTG | |
| | CTGATTCTGGAGTTCGAGCCGACGG | |
| | AGGCCTGGGACAGCACCGGGCCGCA | |
| | CGCGCTGCGCAACGCGATGTTCGCG | |
| | TTCGAAAGTGCCCCCAACCTGCGGG | |
| | CGCTGGCCGAGGTGGCGACCCAGAC | |
| | GGTCCGCGAGCTGACGGGCTTTGAC | |
| | CGGGTGATGCTCTACAAATTTGCCC | |
| | CCGACGCCACCGGCGAAGTGATTGC | |
| | CGAGGCCCGCCGTGAGGGGCTGCAC | |
| | GCCTTTCTGGGCCACCGTTTTCCCG | |
| | CGTCGGACATTCCGGCGCAGGCCCG | |
| | CGCGCTCTACACCCGGCACCTGCTG | |
| | CGCCTGACCGCCGACACCCGCGCCG | |
| | CCACCGTGCCGCTCGATCCCGTCCT | |
| | CAACCCGCAGACGAATGCGCCCACC | |
| | CCGCTGGGCGGCGCCGTGCTGCGCG | |
| | CCACCTCGCCCATGCACATGCAGTA | |
| | CCTGCGGAACATGGGCGTCGGGTCG | |
| | AGCCTGTCGGTGTCGGTGGTGGTCG | |
| | GCGGCCAGCTCTGGGGCCTGATCGC | |
| | CTGCCACCACCAGACGCCCTACGTG | |
| | TTGCCGCCCGACCTGCGAACCACGC | |
| | TCGAATACCTGGGCCGCTTGCTGAG | |
| | CCTGCAAGTTCAGGTCAAGGAAGCG | |
| | GCGGACGTGGCGGCCTTTCGCCAGA | |
| | GCCTGCGGGAGCACCACGCGCGGGT | |
| | GGCCCTCGCGGCGGCGCACTCGCTC | |
| | TCGCCGCACGACACCCTCAGTGACC | |
| | CGGCGCTTGACCTGCTGGGCCTGAT | |
| | GCGGGCCGGGGGCCTGATTCTGCGT | |
| | TTCGAGGGCCGCTGGCAGACGTTGG | |
| | GTGAAGTGCCGCCTGCCCCGGCGGT | |
| | GGACGCGCTGCTGGCGTGGCTCGAA | |
| | ACCCAGCCGGGCGCCCTGGTCCAGA | |
| | CCGACGCGCTAGGCCAACTGTGGCT | |
| | CGCCGGCGCCGATCTCGCCCCCAGC | |
| | GCAGCGGGCCTGCTCGCCATCAGCG | |
| | TGGGCGAGGGCTGGTCGGAGTGCCT | |
| | CGTCTGGCTGCGGCCCGAACTGCGG | |
| | CTGGAGGTCGCCTGGGGCGGGGCCA | |
| | CTCCTGACCAGGCGAAAGACGACCT | |
| | CGGGCCGCGCCACTCATTCGACACC | |
| | TACCTCGAAGAAAAACGCGGCTACG | |
| | CCGAGCCCTGGCATCCCGGCGAAAT | |
| | CGAGGAGGCGCAGGATCTACGTGAC | |
| | ACATTGACCGGGGCGCTGGGCGAGC | |
| | TCGAGTCCATCGTCTCTGCGGTGGT | |
| | TGGCATTCTGCTGGTCGTGGTCTTG | |
| | GGGGTGGTCTTTGGGTCTAGAAAAT | |
| | ATTTACAGAAACCCATGTATGAAGT | |
| | ACAGTGGAAGGTTGTTGAGGAGATA | |
| | AATGGAAACAATTATGTTTACATAG | |
| | ACCCAACACAACTTCCTTATGATCA | |
| | CAAATGGGAGTTTCCCAGAAACAGG | |
| | CTGAGTTTTGGGAAAACCCTGGGTG | |
| | CTGGAGCTTTCGGGAAGGTTGTTGA | |
| | GGCAACTGCTTATGGCTTAATTAAG | |
| | TCAGATGCGGCCATGACTGTCGCTG | |
| | TAAAGATGCTCAAGCCGAGTGCCCA | |
| | TTTGACAGAACGGGAAGCCCTCATG | |
| | TCTGAACTCAAAGTCCTGAGTTACC | |
| | TTGGTAATCACATGAATATTGTGAA | |
| | TCTACTTGGAGCCTGCACCATTGGA | |
| | GGGCCCACCCTGGTCATTACAGAAT | |
| | ATTGTTGCTATGGTGATCTTTTGAA | |
| | TTTTTTGAGAAGAAAACGTGATTCA | |
| | TTTATTTGTTCAAAGCAGGAAGATC | |
| | ATGCAGAAGCTGCACTTTATAAGAA | |
| | TCTTCTGCATTCAAAGGAGTCTTCC | |
| | TGCAGCGATAGTACTAATGAGTACA | |
| | TGGACATGAAACCTGGAGTTTCTTA | |
| | TGTTGTCCCAACCAAGGCCGACAAA | |
| | AGGAGATCTGTGAGAATAGGCTCAT | |
| | ACATAGAAAGAGATGTGACTCCCGC | |
| | CATCATGGAGGATGACGAGTTGGCC | |
| | CTAGACTTAGAAGACTTGCTGAGCT | |
| | TTTCTTACCAGGTGGCAAAGGGCAT | |
| | GGCTTTCCTCGCCTCCAAGAATTGT | |
| | ATTCACAGAGACTTGGCAGCCAGAA | |
| | ATATCCTCCTTACTCATGGTCGGAT | |
| | CACAAAGATTTGTGATTTTGGTCTA | |
| | GCCAGAGACATCAAGAATGATTCTA | |
| | ATTATGTGGTTAAAGGAAACGCTCG | |
| | ACTACCTGTGAAGTGGATGGCACCT | |
| | GAAAGCATTTTCAACTGTGTATACA | |
| | CGTTTGAAAGTGACGTCTGGTCCTA | |
| | TGGGATTTTTCTTTGGGAGCTGTTC | |
| | TCTTTAGGAAGCAGCCCCTATCCTG | |
| | GAATGCCGGTCGATTCTAAGTTCTA | |
| | CAAGATGATCAAGGAAGGCTTCCGG | |
| | ATGCTCAGCCCTGAACACGCACCTG | |
| | CTGAAATGTATGACATAATGAAGAC | |
| | TTGCTGGGATGCAGATCCCCTAAAA | |
| | AGACCAACATTCAAGCAAATTGTTC | |
| | AGCTAATTGAGAAGCAGATTTCAGA | |
| | GAGCACCAATCATATTTACTCCAAC | |
| | TTAGCAAACTGCAGCCCCAACCGAC | |
| | AGAAGCCCGTGGTAGACCATTCTGT | |
| | GCGGATCAATTCTGTCGGCAGCACC | |
| | GCTTCCTCCTCCCAGCCTCTGCTTG | |
| | TGCACGACGATGTCTCTAGAGGCGG | |
| | TGGTGGCAGCGGAGGAGGGGGGAGC | |
| | GGCGGCGGTGGCAGTGGCGGCGGCG | |
| | GTAGTCGCAGCCGCTTTGTGAAAAA | |
| | AGATAGCGCCGGCAGTGCGGGTAGT | |
| | GCGGGGAGCGCCGGTAGCTATCTGG | |
| | CGAACGAAATTCTGTGGGGCAGCGC | |
| | TGGTAGCGCAGGAAGCGCCGGCAGT | |
| | GCAGGGTTTTGCTATGAAAACGAAG | |
| | TGGCGCTGTCATAATCAGCCATACC | |
| | ACATTTG | |
| 51 | ATGGAGACAGACACACTCCTGCTAT | Fusion |
| | GGGTACTGCTGCTCTGGGTTCCAGG | construct |
| | TTCCACTGGTGACAGCAAGGGCGAG | (cMet) |
| | GAGGATAACATGGCCATCATCAAGG | |
| | AGTTCATGCGCTTCAAGGTGCACAT | |
| | GGAGGGCTCCGTGAACGGCCACGAG | |
| | TTCGAGATCGAGGGCGAGGGCGAGG | |
| | GCCGCCCCTACGAGGGCACCCAGAC | |
| | CGCCAAGCTGAAGGTGACCAAGGGT | |
| | GGCCCCCTGCCCTTCGCCTGGGACA | |
| | TCCTGTCCCCTCAGTTCATGTACGG | |
| | CTCCAAGGCCTACGTGAAGCACCCC | |
| | GCCGACATCCCCGACTACTTGAAGC | |
| | TGTCCTTCCCCGAGGGCTTCAAGTG | |
| | GGAGCGCGTGATGAACTTCGAGGAC | |
| | GGCGGCGTGGTGACCGTGACCCAGG | |
| | ACTCCTCCCTGCAGGACGGCGAGTT | |
| | CATCTACAAGGTGAAGCTGCGCGGC | |
| | ACCAACTTCCCCTCCGACGGCCCCG | |
| | TAATGCAGAAGAAGACCATGGGCTG | |
| | GGAGGCCTCCTCCGAGCGGATGTAC | |
| | CCCGAGGACGGCGCCCTGAAGGGCG | |
| | AGATCAAGCAGAGGCTGAAGCTGAA | |
| | GGACGGCGGCCACTACGACGCTGAG | |
| | GTCAAGACCACCTACAAGGCCAAGA | |
| | AGCCCGTGCAGCTGCCCGGCGCCTA | |
| | CAACGTCAACATCAAGTTGGACATC | |
| | ACCTCCCACAACGAGGACTACACCA | |
| | TCGTGGAACAGTACGAACGCGCCGA | |
| | GGGCCGCCACTCCACCGGCGGCATG | |
| | GACGAGCTGTACAAGGAATTCAGTG | |
| | CTGGTAGTGCTGGTAGTGCTGGCAC | |
| | CGGTATGAGCCGGGACCCGTTGCCC | |
| | TTTTTTCCACCGCTTTACCTTGGTG | |
| | GCCCGGAAATTACCACCGAGAACTG | |
| | CGAGCGCGAGCCGATTCATATTCCC | |
| | GGCAGCATCCAGCCGCACGGCGCCC | |
| | TGCTCACTGCCGACGGGCACAGCGG | |
| | CGAGGTGCTCCAGATGAGCCTCAAC | |
| | GCGGCCACTTTTTTGGGACAGGAAC | |
| | CCACAGTGCTGCGCGGACAGACCCT | |
| | CGCCGCACTGCTGCCCGAGCAGTGG | |
| | CCCGCGCTGCAAGCGGCCCTGCCCC | |
| | CCGGCTGCCCCGACGCCCTGCAATA | |
| | CCGCGCAACGCTGGACTGGCCTGCC | |
| | GCCGGGCACCTTTCGCTGACGGTGC | |
| | ACCGGGTCGGCGAGTTGCTGATTCT | |
| | GGAGTTCGAGCCGACGGAGGCCTGG | |
| | GACAGCACCGGGCCGCACGCGCTGC | |
| | GCAACGCGATGTTCGCGTTCGAAAG | |
| | TGCCCCCAACCTGCGGGCGCTGGCC | |
| | GAGGTGGCGACCCAGACGGTCCGCG | |
| | AGCTGACGGGCTTTGACCGGGTGAT | |
| | GCTCTACAAATTTGCCCCCGACGCC | |
| | ACCGGCGAAGTGATTGCCGAGGCCC | |
| | GCCGTGAGGGGCTGCACGCCTTTCT | |
| | GGGCCACCGTTTTCCCGCGTCGGAC | |
| | ATTCCGGCGCAGGCCCGCGCGCTCT | |
| | ACACCCGGCACCTGCTGCGCCTGAC | |
| | CGCCGACACCCGCGCCGCCACCGTG | |
| | CCGCTCGATCCCGTCCTCAACCCGC | |
| | AGACGAATGCGCCCACCCCGCTGGG | |
| | CGGCGCCGTGCTGCGCGCCACCTCG | |
| | CCCATGCACATGCAGTACCTGCGGA | |
| | ACATGGGCGTCGGGTCGAGCCTGTC | |
| | GGTGTCGGTGGTGGTCGGCGGCCAG | |
| | CTCTGGGGCCTGATCGCCTGCCACC | |
| | ACCAGACGCCCTACGTGTTGCCGCC | |
| | CGACCTGCGAACCACGCTCGAATAC | |
| | CTGGGCCGCTTGCTGAGCCTGCAAG | |
| | TTCAGGTCAAGGAAGCGGCGGACGT | |
| | GGCGGCCTTTCGCCAGAGCCTGCGG | |
| | GAGCACCACGCGCGGGTGGCCCTCG | |
| | CGGCGGCGCACTCGCTCTCGCCGCA | |
| | CGACACCCTCAGTGACCCGGCGCTT | |
| | GACCTGCTGGGCCTGATGCGGGCCG | |
| | GGGGCCTGATTCTGCGTTTCGAGGG | |
| | CCGCTGGCAGACGTTGGGTGAAGTG | |
| | CCGCCTGCCCCGGCGGTGGACGCGC | |
| | TGCTGGCGTGGCTCGAAACCCAGCC | |
| | GGGCGCCCTGGTCCAGACCGACGCG | |
| | CTAGGCCAACTGTGGCTCGCCGGCG | |
| | CCGATCTCGCCCCCAGCGCAGCGGG | |
| | CCTGCTCGCCATCAGCGTGGGCGAG | |
| | GGCTGGTCGGAGTGCCTCGTCTGGC | |
| | TGCGGCCCGAACTGCGGCTGGAGGT | |
| | CGCCTGGGGGGGGGCCACTCCTGAC | |
| | CAGGCGAAAGACGACCTCGGGCCGC | |
| | GCCACTCATTCGACACCTACCTCGA | |
| | AGAAAAACGCGGCTACGCCGAGCCC | |
| | TGGCATCCCGGCGAAATCGAGGAGG | |
| | CGCAGGATCTACGTGACACATTGAC | |
| | CGGGGCGCTGGGCGAGTACTTTTAT | |
| | TTCTCCATCGTCTCTGCGGTGGTTG | |
| | GCATTCTGCTGGTCGTGGTCTTGGG | |
| | GGTGGTCTTTGGGTCTAGAAAAAAG | |
| | AGAAAGCAAATTAAAGATCTGGGCA | |
| | GTGAATTAGTTCGCTACGATGCAAG | |
| | AGTACACACTCCTCATTTGGATAGG | |
| | CTTGTAAGTGCCCGAAGTGTAAGCC | |
| | CAACTACAGAAATGGTTTCAAATGA | |
| | ATCTGTAGACTACCGAGCTACTTTT | |
| | CCAGAAGATCAGTTTCCTAATTCAT | |
| | CTCAGAACGGTTCATGCCGACAAGT | |
| | GCAGTATCCTCTGACAGACATGTCC | |
| | CCCATCCTAACTAGTGGGGACTCTG | |
| | ATATATCCAGTCCATTACTGCAAAA | |
| | TACTGTCCACATTGACCTCAGTGCT | |
| | CTAAATCCAGAGCTGGTCCAGGCAG | |
| | TGCAGCATGTAGTGATTGGGCCCAG | |
| | TAGCCTGATTGTGCATTTCAATGAA | |
| | GTCATAGGAAGAGGGCATTTTGGTT | |
| | GTGTATATCATGGGACTTTGTTGGA | |
| | CAATGATGGCAAGAAAATTCACTGT | |
| | GCTGTGAAATCCTTGAACAGAATCA | |
| | CTGACATAGGAGAAGTTTCCCAATT | |
| | TCTGACCGAGGGAATCATCATGAAA | |
| | GATTTTAGTCATCCCAATGTCCTCT | |
| | CGCTCCTGGGAATCTGCCTGCGAAG | |
| | TGAAGGGTCTCCGCTGGTGGTCCTA | |
| | CCATACATGAAACATGGAGATCTTC | |
| | GAAATTTCATTCGAAATGAGACTCA | |
| | TAATCCAACTGTAAAAGATCTTATT | |
| | GGCTTTGGTCTTCAAGTAGCCAAAG | |
| | GCATGAAATATCTTGCAAGCAAAAA | |
| | GTTTGTCCACAGAGACTTGGCTGCA | |
| | AGAAACTGTATGCTGGATGAAAAAT | |
| | TCACAGTCAAGGTTGCTGATTTTGG | |
| | TCTTGCCAGAGACATGTATGATAAA | |
| | GAATACTATAGTGTACACAACAAAA | |
| | CAGGTGCAAAGCTGCCAGTGAAGTG | |
| | GATGGCTTTGGAAAGTCTGCAAACT | |
| | CAAAAGTTTACCACCAAGTCAGATG | |
| | TGTGGTCCTTTGGCGTGCTCCTCTG | |
| | GGAGCTGATGACAAGAGGAGCCCCA | |
| | CCTTATCCTGACGTAAACACCTTTG | |
| | ATATAACTGTTTACTTGTTGCAAGG | |
| | GAGAAGACTCCTACAACCCGAATAC | |
| | TGCCCAGACCCCTTATATGAAGTAA | |
| | TGCTAAAATGCTGGCACCCTAAAGC | |
| | CGAAATGCGCCCATCCTTTTCTGAA | |
| | CTGGTGTCCCGGATATCAGCGATCT | |
| | TCTCTACTTTCATTGGGGAGCACTA | |
| | TGTCCATGTGAACGCTACTTATGTG | |
| | AACGTAAAATGTGTCGCTCCGTATC | |
| | CTTCTCTGTTGTCATCAGAAGATAA | |
| | CGCTGATGATGAGGTGGACACACGA | |
| | CCAGCCTCCTTCTGGGAGACATCTA | |
| | GAGGCGGTGGTGGCAGCGGAGGAGG | |
| | GGGGAGCGGCGGCGGTGGCAGTGGC | |
| | GGCGGCGGTAGTCGCAGCCGCTTTG | |
| | TGAAAAAAGATAGCGCCGGCAGTGC | |
| | GGGTAGTGCGGGGAGCGCCGGTAGC | |
| | TATCTGGCGAACGAAATTCTGTGGG | |
| | GCAGCGCTGGTAGCGCAGGAAGCGC | |
| | CGGCAGTGCAGGGTTTTGCTATGAA | |
| | AACGAAGTGGCGCTGTCATAATCAG | |
| | CCATACCACATTTG | |
| 52 | ATGGAGACAGACACACTCCTGCTAT | Fusion |
| | GGGTACTGCTGCTCTGGGTTCCAGG | construct (IR1) |
| | TTCCACTGGTGACAGCAAGGGCGAG | |
| | GAGGATAACATGGCCATCATCAAGG | |
| | AGTTCATGCGCTTCAAGGTGCACAT | |
| | GGAGGGCTCCGTGAACGGCCACGAG | |
| | TTCGAGATCGAGGGCGAGGGCGAGG | |
| | GCCGCCCCTACGAGGGCACCCAGAC | |
| | CGCCAAGCTGAAGGTGACCAAGGGT | |
| | GGCCCCCTGCCCTTCGCCTGGGACA | |
| | TCCTGTCCCCTCAGTTCATGTACGG | |
| | CTCCAAGGCCTACGTGAAGCACCCC | |
| | GCCGACATCCCCGACTACTTGAAGC | |
| | TGTCCTTCCCCGAGGGCTTCAAGTG | |
| | GGAGCGCGTGATGAACTTCGAGGAC | |
| | GGCGGCGTGGTGACCGTGACCCAGG | |
| | ACTCCTCCCTGCAGGACGGCGAGTT | |
| | CATCTACAAGGTGAAGCTGCGCGGC | |
| | ACCAACTTCCCCTCCGACGGCCCCG | |
| | TAATGCAGAAGAAGACCATGGGCTG | |
| | GGAGGCCTCCTCCGAGCGGATGTAC | |
| | CCCGAGGACGGCGCCCTGAAGGGCG | |
| | AGATCAAGCAGAGGCTGAAGCTGAA | |
| | GGACGGCGGCCACTACGACGCTGAG | |
| | GTCAAGACCACCTACAAGGCCAAGA | |
| | AGCCCGTGCAGCTGCCCGGCGCCTA | |
| | CAACGTCAACATCAAGTTGGACATC | |
| | ACCTCCCACAACGAGGACTACACCA | |
| | TCGTGGAACAGTACGAACGCGCCGA | |
| | GGGCCGCCACTCCACCGGCGGCATG | |
| | GACGAGCTGTACAAGGAATTCAGTG | |
| | CTGGTAGTGCTGGTAGTGCTGGCAC | |
| | CGGTATGAGCCGGGACCCGTTGCCC | |
| | TTTTTTCCACCGCTTTACCTTGGTG | |
| | GCCCGGAAATTACCACCGAGAACTG | |
| | CGAGCGCGAGCCGATTCATATTCCC | |
| | GGCAGCATCCAGCCGCACGGCGCCC | |
| | TGCTCACTGCCGACGGGCACAGCGG | |
| | CGAGGTGCTCCAGATGAGCCTCAAC | |
| | GCGGCCACTTTTTTGGGACAGGAAC | |
| | CCACAGTGCTGCGCGGACAGACCCT | |
| | CGCCGCACTGCTGCCCGAGCAGTGG | |
| | CCCGCGCTGCAAGCGGCCCTGCCCC | |
| | CCGGCTGCCCCGACGCCCTGCAATA | |
| | CCGCGCAACGCTGGACTGGCCTGCC | |
| | GCCGGGCACCTTTCGCTGACGGTGC | |
| | ACCGGGTCGGCGAGTTGCTGATTCT | |
| | GGAGTTCGAGCCGACGGAGGCCTGG | |
| | GACAGCACCGGGCCGCACGCGCTGC | |
| | GCAACGCGATGTTCGCGTTCGAAAG | |
| | TGCCCCCAACCTGCGGGCGCTGGCC | |
| | GAGGTGGCGACCCAGACGGTCCGCG | |
| | AGCTGACGGGCTTTGACCGGGTGAT | |
| | GCTCTACAAATTTGCCCCCGACGCC | |
| | ACCGGCGAAGTGATTGCCGAGGCCC | |
| | GCCGTGAGGGGCTGCACGCCTTTCT | |
| | GGGCCACCGTTTTCCCGCGTCGGAC | |
| | ATTCCGGCGCAGGCCCGCGCGCTCT | |
| | ACACCCGGCACCTGCTGCGCCTGAC | |
| | CGCCGACACCCGCGCCGCCACCGTG | |
| | CCGCTCGATCCCGTCCTCAACCCGC | |
| | AGACGAATGCGCCCACCCCGCTGGG | |
| | CGGCGCCGTGCTGCGCGCCACCTCG | |
| | CCCATGCACATGCAGTACCTGCGGA | |
| | ACATGGGCGTCGGGTCGAGCCTGTC | |
| | GGTGTCGGTGGTGGTCGGCGGCCAG | |
| | CTCTGGGGCCTGATCGCCTGCCACC | |
| | ACCAGACGCCCTACGTGTTGCCGCC | |
| | CGACCTGCGAACCACGCTCGAATAC | |
| | CTGGGCCGCTTGCTGAGCCTGCAAG | |
| | TTCAGGTCAAGGAAGCGGCGGACGT | |
| | GGCGGCCTTTCGCCAGAGCCTGCGG | |
| | GAGCACCACGCGCGGGTGGCCCTCG | |
| | CGGCGGCGCACTCGCTCTCGCCGCA | |
| | CGACACCCTCAGTGACCCGGCGCTT | |
| | GACCTGCTGGGCCTGATGCGGGCCG | |
| | GGGGCCTGATTCTGCGTTTCGAGGG | |
| | CCGCTGGCAGACGTTGGGTGAAGTG | |
| | CCGCCTGCCCCGGCGGTGGACGCGC | |
| | TGCTGGCGTGGCTCGAAACCCAGCC | |
| | GGGCGCCCTGGTCCAGACCGACGCG | |
| | CTAGGCCAACTGTGGCTCGCCGGCG | |
| | CCGATCTCGCCCCCAGCGCAGCGGG | |
| | CCTGCTCGCCATCAGCGTGGGCGAG | |
| | GGCTGGTCGGAGTGCCTCGTCTGGC | |
| | TGCGGCCCGAACTGCGGCTGGAGGT | |
| | CGCCTGGGGGGGGGCCACTCCTGAC | |
| | CAGGCGAAAGACGACCTCGGGCCGC | |
| | GCCACTCATTCGACACCTACCTCGA | |
| | AGAAAAACGCGGCTACGCCGAGCCC | |
| | TGGCATCCCGGCGAAATCGAGGAGG | |
| | CGCAGGATCTACGTGACACATTGAC | |
| | CGGGGCGCTGGGCGAGCTCGAGTCC | |
| | ATCGTCTCTGCGGTGGTTGGCATTC | |
| | TGCTGGTCGTGGTCTTGGGGGTGGT | |
| | CTTTGGGTCTAGAAGAAAGAGGCAG | |
| | CCAGATGGGCCGCTGGGACCGCTTT | |
| | ACGCTTCTTCAAACCCTGAGTATCT | |
| | CAGTGCCAGTGATGTGTTTCCATGC | |
| | TCTGTGTACGTGCCGGACGAGTGGG | |
| | AGGTGTCTCGAGAGAAGATCACCCT | |
| | CCTTCGAGAGCTGGGGCAGGGCTCC | |
| | TTCGGCATGGTGTATGAGGGCAATG | |
| | CCAGGGACATCATCAAGGGTGAGGC | |
| | AGAGACCCGCGTGGCGGTGAAGACG | |
| | GTCAACGAGTCAGCCAGTCTCCGAG | |
| | AGCGGATTGAGTTCCTCAATGAGGC | |
| | CTCGGTCATGAAGGGCTTCACCTGC | |
| | CATCACGTGGTGCGCCTCCTGGGAG | |
| | TGGTGTCCAAGGGCCAGCCCACGCT | |
| | GGTGGTGATGGAGCTGATGGCTCAC | |
| | GGAGACCTGAAGAGCTACCTCCGTT | |
| | CTCTGCGGCCAGAGGCTGAGAATAA | |
| | TCCTGGCCGCCCTCCCCCTACCCTT | |
| | CAAGAGATGATTCAGATGGCGGCAG | |
| | AGATTGCTGACGGGATGGCCTACCT | |
| | GAACGCCAAGAAGTTTGTGCATCGG | |
| | GACCTGGCAGCGAGAAACTGCATGG | |
| | TCGCCCATGATTTTACTGTCAAAAT | |
| | TGGAGACTTTGGAATGACCAGAGAC | |
| | ATCTATGAAACGGATTACTACCGGA | |
| | AAGGGGGCAAGGGTCTGCTCCCTGT | |
| | ACGGTGGATGGCACCGGAGTCCCTG | |
| | AAGGATGGGGTCTTCACCACTTCTT | |
| | CTGACATGTGGTCCTTTGGCGTGGT | |
| | CCTTTGGGAAATCACCAGCTTGGCA | |
| | GAACAGCCTTACCAAGGCCTGTCTA | |
| | ATGAACAGGTGTTGAAATTTGTCAT | |
| | GGATGGAGGGTATCTGGATCAACCC | |
| | GACAACTGTCCAGAGAGAGTCACTG | |
| | ACCTCATGCGCATGTGCTGGCAATT | |
| | CAACCCCAAGATGAGGCCAACCTTC | |
| | CTGGAGATTGTCAACCTGCTCAAGG | |
| | ACGACCTGCACCCCAGCTTTCCAGA | |
| | GGTGTCGTTCTTCCACAGCGAGGAG | |
| | AACAAGGCTCCCGAGAGTGAGGAGC | |
| | TGGAGATGGAGTTTGAGGACATGGA | |
| | GAATGTGCCCCTGGACCGTTCCTCG | |
| | CACTGTCAGAGGGAGGAGGCGGGGG | |
| | GCCGGGATGGAGGGTCCTCGCTGGG | |
| | TTTCAAGCGGAGCTACGAGGAACAC | |
| | ATCCCTTACACACACATGAACGGAG | |
| | GCAAGAAAAACGGGCGGATTCTGAC | |
| | CTTGCCTCGGTCCAATCCTTCCTCT | |
| | AGAGGCGGTGGTGGCAGCGGAGGAG | |
| | GGGGGAGCGGCGGCGGTGGCAGTGG | |
| | CGGCGGCGGTAGTCGCAGCCGCTTT | |
| | GTGAAAAAAGATAGCGCCGGCAGTG | |
| | CGGGTAGTGCGGGGAGCGCCGGTAG | |
| | CTATCTGGCGAACGAAATTCTGTGG | |
| | GGCAGCGCTGGTAGCGCAGGAAGCG | |
| | CCGGCAGTGCAGGGTTTTGCTATGA | |
| | AAACGAAGTGGCGCTGTCATAATCA | |
| | GCCATACCACATTTG | |

In some embodiments, the receptor is a receptor tyrosine kinase (RTK). Non-limiting examples of receptor tyrosine kinases include EGFR (e.g., EGFR/HER1/ErbB1, HER2/Neu/ErbB2, HER3/ErbB3, HER4/ErbB4), INSR (insulin receptor), FGFR1, IGF-IR, IGF-IIIR, IRR (insulin receptor-related receptor), PDGFR (e.g., PDGFRA, PDGFRB), cKIT/SCFR, VEGFR-1/FLT-1, VEGFR-2/FLK-1/KDR, VEGFR-3/FLT-4, FLT-3/FLK-2, CSF-1R, FGFR 1-4, CCK4, Trk A-C, MET (e.g., cMet), RON, EPHA 1-8, EPHB 1-6, AXL, MER, TYRO3, TIE, TEK, RYK, DDR 1-2, RET, c-ROS, LTK (leukocyte tyrosine kinase), ALK (anaplastic lymphoma kinase), ROR 1-2, MUSK, AATYK 1-3, Insulin receptor (IR1), and RTK 106.

In some embodiments, the receptor tyrosine kinase is selected from EGFR, HER2, FGFR1, TrkA, TrkB, cKIT, cMet, and IR1.

In some embodiments, the transmembrane domain comprises a transmembrane domain of the receptor tyrosine kinase or a variant thereof. In some embodiments, the transmembrane domain comprises a transmembrane domain of EGFR, HER2, or a variant thereof. In some embodiments, the transmembrane domain further comprises one or more repeats of Tyrosine (Y)-Phenylalanine (F), as represented by [YF]n, where n is an integer from 1 to 10 (e.g., 1, 2, 3, 4, 5, 6, 7, 8, 9, 10).

In some embodiments, the transmembrane domain comprises an amino acid sequence having at least 90% sequence identity to the amino acid sequence of any one of SEQ ID NOs: 2-3 or comprises the amino acid sequence of any one of SEQ ID NOs: 2-3.

In some embodiments, the intracellular domain may further comprise a juxtamembrane domain and a catalytic domain (e.g., tyrosine kinase domain).

The term “juxtamembrane domain” or “juxtamembrane region,” as used herein, refers to the region that connects the transmembrane domain (e.g., transmembrane helix) to the catalytic domain (e.g., tyrosine kinase domain).

In some embodiments, the intracellular domain is a tyrosine kinase domain of a second receptor tyrosine kinase. Non-limiting examples of the second receptor tyrosine kinases include EGFR (e.g., EGFR/HER1/ErbB1, HER2/Neu/ErbB2, HER3/ErbB3, HER4/ErbB4), INSR (insulin receptor), FGFR1, IGF-IR, IGF-IIIR, IRR (insulin receptor-related receptor), PDGFR (e.g., PDGFRA, PDGFRB), cKIT/SCFR, VEGFR-1/FLT-1, VEGFR-2/FLK-1/KDR, VEGFR-3/FLT-4, FLT-3/FLK-2, CSF-1R, FGFR 1-4, CCK4, Trk A-C, MET (e.g., cMet), RON, EPHA 1-8, EPHB 1-6, AXL, MER, TYRO3, TIE, TEK, RYK, DDR 1-2, RET, c-ROS, LTK (leukocyte tyrosine kinase), ALK (anaplastic lymphoma kinase), ROR 1-2, MUSK, AATYK 1-3, IR1, and RTK 106.

In some embodiments, the second receptor tyrosine kinase is selected from EGFR, HER2, FGFR1, TrkA, TrkB, cKIT, cMet, and IR1.

In some embodiments, the intracellular domain comprises an amino acid sequence having at least 80% (e.g., 80%, 85%, 90%, 91%, 92%, 93%, 94%, 95%, 96%, 97%, 98%, 99%) sequence identity to the amino acid sequence of any one of SEQ ID NOs: 4-7 or comprises the amino acid sequence of any one of SEQ ID NOs: 4-7.

In some embodiments, the chimeric polypeptide further comprises a signaling peptide linked to the N-terminus of the light-responsive polypeptide. In some embodiments, the signaling peptide comprises an Igκ signaling peptide.

In some embodiments, the signaling peptide comprises an amino acid sequence having at least 80% (e.g., 80%, 85%, 90%, 91%, 92%, 93%, 94%, 95%, 96%, 97%, 98%, 99%) sequence identity to the amino acid sequence of SEQ ID NO: 16 or comprises the amino acid sequence of SEQ ID NO: 16

In some embodiments, the chimeric polypeptide further comprises a Golgi-export peptide linked to the C-terminus of the intracellular domain. In some embodiments, the Golgi-export peptide comprises an amino acid sequence having at least 90% sequence identity to the amino acid sequence of any one of SEQ ID NOs: 17-19 or comprises the amino acid sequence of any one of SEQ ID NOs: 17-19.

In some embodiments, the chimeric polypeptide comprises an amino acid sequence having (e.g., 80%, 85%, 90%, 91%, 92%, 93%, 94%, 95%, 96%, 97%, 98%, 99%) sequence identity to the amino acid sequence of any one of SEQ ID NOs: 20-27 or comprises the amino acid sequence of any one of SEQ ID NOs: 20-27.

In some embodiments, the light-responsive polypeptide is linked to the transmembrane domain via a linker, e.g., a peptide linker. In some embodiments, the transmembrane domain is linked to the intracellular domain via a linker, e.g., a peptide linker.

The term “linker” refers to any means, entity, or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as a platinum atom. For covalent linkages, various functionalities can be used, such as amide groups, including carbonic acid derivatives, ethers, esters, including organic and inorganic esters, amino, urethane, urea, and the like. To provide for linking, the domains can be modified by oxidation, hydroxylation, substitution, reduction etc., to provide a site for coupling. Methods for conjugation are well known by persons skilled in the art and are encompassed for use in the present disclosure. Linker moieties include, but are not limited to, chemical linker moieties, or for example, a peptide linker moiety (a linker sequence).

A peptide linker can range from 2 amino acids to 60 or more amino acids, and in some embodiments, a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length. In some embodiments, a peptide linker is at least 5 amino acids, at least 6 amino acids or at least 7 amino acids in length and optionally is up to 30 amino acids, up 20) to 40 amino acids, up to 50 amino acids or up to 60 amino acids in length. In some embodiments, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

In some embodiments, the linker comprises polar (e.g., serine(S)) or charged (e.g., lysine (K)) residues. In some embodiments, the linker is a flexible linker, e.g., comprising one or more glycine (G) or serine(S) residues.

Examples of flexible linkers that can be used in the fusion protein of the disclosure include those disclosed by Chen et al., 2013, Adv Drug Deliv Rev. 65 (10): 1357-1369 and Klein et al., 2014, Protein Engineering, Design & Selection 27 (10): 325-330. Particularly useful flexible linkers are or comprise repeats of glycines and serines, e.g., represented by [Ser]m[Gly]n, where m or n is an integer from 1 to 20 (e.g., 1, 2, 3, 4, 5, 6, 7, 8, 9, 10, 11, 12, 13, 14, 15, 16, 17, 18, 19, 20). Polyglycine linkers can suitably be used in the fusion protein of the disclosure. In some embodiments, a peptide linker comprises two or more consecutive glycines, represented by [Gly]n where n is an integer from 1 to 20 (e.g., 1, 2, 3, 4, 5, 6, 7, 8, 9, 10, 11, 12, 13, 14, 15, 16, 17, 18, 19, 20).

In some embodiments, the wavelength is in far-red or near-infrared spectrum. In some embodiments, the wavelength is from about 650 nm to about 900 nm (e.g., 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm). In some embodiments, the wavelength is from about 650 nm to about 700 nm (e.g., 650 nm, 655 nm, 660 nm, 665 nm, 670 nm, 675 nm, 680 nm, 685 nm, 690 nm, 695 nm, 700 nm). In some embodiments, the wavelength is from about 700 nm to about 780 nm (e.g., 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775 nm, 780 nm).

As used herein, “infrared” or “near-infrared” or “infrared light” or “near-infrared light” refers to electromagnetic radiation in the spectrum immediately above that of visible light, measured from the nominal edge of visible red light at 0.74 mh, and extending to 300 mh. These wavelengths correspond to a frequency range of approximately 1 to 400 THz. In particular, “near-infrared” or “near-infrared light” also refers to electromagnetic radiation measuring 0.75-1.4 m in wavelength, defined by the water absorption. “Visible light” is defined as electromagnetic radiation with wavelengths between 380 nm and 750 nm. In general, “electromagnetic radiation,” including light, is generated by the acceleration and deceleration or changes in movement (vibration) of electrically charged particles, such as parts of molecules (or adjacent atoms) with high thermal energy, or electrons in atoms (or molecules).

b. Chimeric Polypeptides

In another aspect, this disclosure also provides a polypeptide comprising (a) an extracellular light-responsive polypeptide, (b) a transmembrane domain linked to the C-terminus of the light-responsive polypeptide, and (c) an intracellular domain of a receptor linked to the C-terminus of the transmembrane domain, wherein the light-responsive polypeptide, when associated with a chromophore, is capable of switching from a first state to a second state when exposed to illumination by a wavelength, and wherein the intracellular domain of the receptor is activated at the second state.

In some embodiments, the polypeptide is encoded by a polynucleotide disclosed herein. In some embodiments, the polypeptide comprises an amino acid sequence having at least 80% (80%, 85%, 90%, 91%, 92%, 93%, 94%, 95%, 96%, 97%, 98%, 99%) sequence identity to the amino acid sequence of any one of SEQ ID NOs: 20-27 or comprises the amino acid sequence of any one of SEQ ID NOs: 20-27.

In some embodiments, the light-responsive polypeptide is linked to the transmembrane domain via a linker, e.g., a peptide linker or a non-peptide linker. In some embodiments, the transmembrane domain is linked to the intracellular domain via a linker, e.g., a peptide linker or a non-peptide linker.

A peptide linker can range from 2 amino acids to 60 or more amino acids, and in some embodiments, a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length. In some embodiments, a peptide linker is at least 5 amino acids, at least 6 amino acids or at least 7 amino acids in length and optionally is up to 30 amino acids, up to 40 amino acids, up to 50 amino acids or up to 60 amino acids in length. In some embodiments, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

In some embodiments, the linker comprises polar (e.g., serine(S)) or charged (e.g., lysine (K)) residues. In some embodiments, the linker is a flexible linker, e.g., comprising one or more glycine (G) or serine(S) residues. In some embodiments, the linker comprises polar (e.g., serine(S)) or charged (e.g., lysine (K)) residues. In some embodiments, the linker is a flexible linker, e.g., comprising one or more glycine (G) or serine(S) residues.

Examples of flexible linkers that can be used in the fusion protein of the disclosure include those disclosed by Chen et al., 2013, Adv Drug Deliv Rev. 65 (10): 1357-1369 and Klein et al., 2014, Protein Engineering, Design & Selection 27 (10): 325-330. Particularly useful flexible linkers are or comprise repeats of glycines and serines, e.g., represented by [Ser]m[Gly]n, where m or n is an integer from 1 to 20 (e.g., 1, 2, 3, 4, 5, 6, 7, 8, 9, 10, 11, 12, 13, 14, 15, 16, 17, 18, 19, 20). Polyglycine linkers can suitably be used in the fusion protein of the disclosure. In some embodiments, a peptide linker comprises two or more consecutive glycines, represented by [Gly]n where n is an integer from 1 to 20 (e.g., 1, 2, 3, 4, 5, 6, 7, 8, 9, 10, 11, 12, 13, 14, 15, 16, 17, 18, 19, 20).

In some embodiments, the linker is a non-peptide linker. As used herein, the term “non-peptide linker” refers to a biocompatible polymer composed of two or more repeating units linked to each other, in which the repeating units are linked to each other by any non-peptide covalent bond. This non-peptidyl linker may have two ends or three ends. Examples of the non-peptidyl linker may include, without limitation, polyethylene glycol, polypropylene glycol, a copolymer of ethylene glycol with propylene glycol, polyoxyethylated polyol, polyvinyl alcohol, polysaccharide, dextran, polyvinyl ethyl ether, biodegradable polymers such as polylactic acid (PLA), and polylactic-glycolic acid (PLGA), lipid polymers, chitins, hyaluronic acid, and combinations thereof.

In some embodiments, the wavelength is in far-red or near-infrared spectrum. In some embodiments, the wavelength is from about 650 nm to about 900 nm (e.g., 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm). In some embodiments, the wavelength is from about 650 nm to about 700 nm (e.g., 650 nm, 655 nm, 660 nm, 665 nm, 670 nm, 675 nm, 680 nm, 685 nm, 690 nm, 695 nm, 700 nm). In some embodiments, the wavelength is from about 700 nm to about 780 nm (e.g., 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775 nm, 780 nm).

In some embodiments, the light-responsive polypeptide may be conjugated or linked to a detectable tag or a detectable marker (e.g., a radionuclide, a fluorescent dye). In some embodiments, the detectable tag can be an affinity tag. The term “affinity tag” as used herein relates to a moiety attached to a polypeptide, which allows the polypeptide to be purified from a biochemical mixture. Affinity tags can consist of amino acid sequences or can include amino acid sequences to which chemical groups are attached by post-translational modifications. Non-limiting examples of affinity tags include His-tag, CBP-tag (CBP: calmodulin-binding protein), CYD-tag (CYD: covalent yet dissociable NorpD peptide), Strep-tag, StrepII-tag, FLAG-tag, HPC-tag (HPC: heavy chain of protein C), GST-tag (GST: glutathione S transferase), Avi-tag, biotinylated tag, Myc-tag, a myc-myc-hexahistidine (mmh) tag 3×FLAG tag, a SUMO tag, MBP-tag (MBP: maltose-binding protein), Alfa-tag, Sun-tag, and Moon-tag. Further examples of affinity tags can be found in Kimple et al., Curr Protoc Protein Sci. 2013 Sep. 24; 73: Unit 9.9.

In some embodiments, the detectable tag can be conjugated or linked to the N- and/or C-terminus of the light-responsive polypeptide. The detectable tag and the affinity tag may also be separated by one or more amino acids. In some embodiments, the detectable tag can be conjugated or linked to the light-responsive polypeptide via a cleavable element. In the context of the present disclosure, the term “cleavable element” relates to peptide sequences that are susceptible to cleavage by chemical agents or enzymatic means, such as proteases. Proteases may be sequence-specific (e.g., thrombin) or may have limited sequence specificity (e.g., trypsin). Cleavable elements I and II may also be included in the amino acid sequence of a detection tag or polypeptide, particularly where the last amino acid of the detection tag or polypeptide is K or R.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

In another aspect, this disclosure provides a polynucleotide encoding a polypeptide described above. In some embodiments, the polypeptide can be encoded by a single nucleic acid or by a plurality (e.g., two, three, four or more) nucleic acids. The nucleic acids of the disclosure can be DNA or RNA (e.g., mRNA).

c. Vectors, Cells, and Compositions

In another aspect, this disclosure provides a vector comprising a polynucleotide disclosed herein. The term “vector” or “expression vector” is synonymous with “expression construct” and refers to a nucleic acid molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector may comprise an expression cassette. Expression vectors allow transcription of large amounts of stable mRNA. Once the expression vector is inside the target cell, the ribonucleic acid molecule or protein that is encoded by the gene is produced by the cellular transcription and/or translation machinery.

The vectors may comprise a polynucleotide that encodes an RNA (e.g., RNAi, ribozymes, miRNA, siRNA) that when transcribed from the polynucleotides of the vector will result in the accumulation of light-responsive chimeric proteins on the plasma membranes of target cells. Vectors which may be used, include, without limitation, lentiviral, HSV, and adenoviral vectors. Lentiviruses include, but are not limited to, HIV-1, HIV-2, SIV, FIV, and EIAV. Lentiviruses may be pseudotyped with the envelope proteins of other viruses, including, but not limited to VSV, 20) rabies, Mo-MLV, baculovirus, and Ebola. Such vectors may be prepared using standard methods in the art.

In some embodiments, the vector is a recombinant AAV vector. AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced, and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, which contains the rep gene involved in viral replication and expression of the viral genes; and the right-hand part of the genome, which contains the cap gene encoding the capsid proteins of the virus.

The application of AAV, for example, as a vector for gene therapy, has been rapidly developed in recent years. Wild-type AAV can infect, with a comparatively high titer, dividing or non-dividing cells, or tissues of a mammal, including human, and also can integrate into human cells at a specific site (on the long arm of chromosome 19) (Kotin, R. M., et al., Proc. Natl. Acad. Sci. USA 87:2211-2215, 1990) (Samulski, R. J, et al., EMBO J. 10:3941-3950, 1991 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not hitherto been found to be associated with any human disease, nor any change of biological characteristics arising from the integration has been observed. There are sixteen serotypes of AAV reported in the literature, respectively named AAV1, AAV2, AAV3, AAV4, AAV5, AAV6, AAV7, AAV8, AAV9, AAV10, AAV11, AAV12, AAV13, AAV14, AAV15, AAV16, and recombinant variants thereof, wherein AAV5 is originally isolated from humans (Bantel-Schaal, and H. zur Hausen. 1984. Virology 134:52-63), while AAV1-4 and AAV6 are all found in the study of adenovirus (Ursula Bantel-Schaal, Hajo Delius and Harald zur Hausen. J. Virol. 1999, 73:939-947). 20)

AAV vectors may be prepared using standard methods in the art. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Hudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors,” the disclosures of which are herein incorporated by reference in their entireties. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See, e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entireties). These publications describe various AAV-derived constructs in which the rep and/or cap genes are deleted and replaced by a gene of interest and the use of these constructs for transferring the gene of interest in vitro (into cultured cells) or in vivo (directly into an organism). The replication-defective recombinant AAVs can be prepared by co-transfecting a plasmid containing the nucleic acid sequence of interest flanked by two AAV inverted terminal repeat (ITR) regions and a plasmid carrying the AAV encapsidation genes (rep and cap genes) into a cell line that is infected with a human helper virus (for example an adenovirus). The AAV recombinants that are produced are then purified by standard techniques.

In some embodiments, the vector(s) can be encapsidated into a virus particle (e.g., AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, also provided is a recombinant virus particle (recombinant because it contains a recombinant polynucleotide) comprising any of the vectors described herein. Methods of producing such particles are known in the art and are described in U.S. Pat. No. 6,596,535.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. Methods and conditions for culturing the resulting transfected cells and for recovering the expressed polypeptides are known to those skilled in the art and may be varied or optimized depending upon the specific expression vector and mammalian host cell employed, based upon the present description.

In some embodiments, the vector comprises a viral vector. In some embodiments, the viral vector comprises an adeno-associated viral vector, lentiviral vector or adenoviral vector. In some embodiments, the adeno-associated viral vector is selected from AAV1, AAV2, AAV3, AAV4, AAV5, AAV6, AAV7, AAV8, AAV9, AAV10, AAV11, AAV 12, AAV13, AAV rh74, and recombinant subtypes thereof.

The disclosure also provides host cells comprising a nucleic acid of the disclosure. In one embodiment, the host cells are genetically engineered to comprise one or more nucleic acids described herein. In one embodiment, the host cells are genetically engineered by using an expression cassette. The phrase “expression cassette” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The cell can be, but is not limited to, a eukaryotic cell, an insect cell, or a mammalian cell (e.g., a human cell). Suitable eukaryotic cells include, but are not limited to, Vero cells, HeLa cells, COS cells, CHO cells, HEK293 cells, BHK cells, and MDCKII cells. Suitable insect cells include, but are not limited to, Sf9 cells.

In another aspect, the above-described polynucleotide, vector, polypeptide, or cell can be incorporated into compositions. The composition may further include a pharmaceutically acceptable carrier. The pharmaceutical compositions are generally formulated in full compliance with all Good Manufacturing Practice (GMP) regulations of the U.S. Food and Drug Administration.

The terms “pharmaceutically acceptable,” “physiologically tolerable,” as referred to compositions, carriers, diluents, and reagents, are used interchangeably and include materials that are capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable and includes excipients that are acceptable for veterinary use as well as for human pharmaceutical use.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the disclosed composition, use thereof in the compositions is contemplated. In some embodiments, a second therapeutic agent, such as an anti-cancer or anti-tumor, can also be incorporated into pharmaceutical compositions.

Also provided in this disclosure is a kit comprising a polynucleotide, a vector, a polypeptide, a cell, or a composition, as described above. The components of the kit may be provided in any form, e.g., liquid, dried or lyophilized form, preferably substantially pure and/or sterile. When the components of the kit are provided in a liquid solution, the liquid solution preferably is an aqueous solution. When the agents are provided as a dried form, reconstitution generally is by the addition of a suitable solvent and acidulant. The acidulant and solvent, e.g., an aprotic solvent, sterile water, or a buffer, can optionally be provided in the kit. In some embodiments, the kit may further include informational materials. The informational material of the kits is not limited in its form. For example, the informational material can include information about the production of the composition, concentration, date of expiration, batch or production site information, and so forth. In addition to the composition, the kit can include other ingredients, such as a solvent or buffer, an adjuvant, a stabilizer, or a preservative.

### B. Methods of Use

a. Methods of Modulating Receptor Activity and Gene Expression

In another aspect, this disclosure further provides a method for modulating (e.g., inhibiting, activating) an expression level of a gene in a cell. The method comprises: (a) introducing to the cell a polynucleotide or a vector, as disclosed herein; and exposing the cell to illumination by an activation wavelength to modulate the expression level of the gene; or (b) providing a polypeptide or a cell, as disclosed herein; and exposing the polypeptide or the cell to illumination by the activation wavelength to modulate the expression level of the gene.

In another aspect, this disclosure also provides a method for modulating (e.g., inhibiting, activating) an expression level of a gene in a subject. The method comprises: introducing to the subject a polynucleotide, a vector, a composition thereof, as disclosed herein; and exposing the subject to illumination by an activation wavelength to modulate the expression level of the gene in the subject. In some embodiments, the subject is exposed to illumination at a site of the subject where modulation of the expression level of the gene is needed.

The polynucleotide or vector or a composition thereof can be introduced to the subject via one or more routes of administration using one or more of a variety of methods known in the art. As will be appreciated by the skilled artisan, the route and/or mode of administration will vary depending upon the desired results. For example, administration for the polynucleotide or vector or a composition thereof may include intravenous, intramuscular, intradermal, intraperitoneal, subcutaneous, spinal or other parenteral routes of administration, for example, by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal injection and infusion. Alternatively, the polynucleotide or vector or a composition thereof can be administered via a non-parenteral route, such as a topical, epidermal or mucosal route of administration, for example, intranasally, orally, vaginally, rectally, sublingually or topically.

As used herein, the term “modulate” is meant to refer to any change in biological state, i.e. increasing, decreasing, and the like.

The terms “decrease,” “reduced,” “reduction,” “decrease,” or “inhibit” are all used herein generally to mean a decrease by a statistically significant amount. However, for avoidance of doubt, “reduced,” “reduction” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

The terms “increased,” “increase” or “enhance” or “activate” are all used herein to generally mean an increase by a statically significant amount; for the avoidance of any doubt, the terms “increased,” “increase,” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase 30 between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

In some embodiments, the gene is regulated by a receptor tyrosine kinase. Non-limiting examples of receptor tyrosine kinases include EGFR (e.g., EGFR/HER1/ErbB1, HER2/Neu/ErbB2, HER3/ErbB3, HER4/ErbB4), INSR (insulin receptor), FGFR1, IGF-IR, IGF-IIIR, IRR (insulin receptor-related receptor), PDGFR (e.g., PDGFRA, PDGFRB), cKIT/SCFR, VEGFR-1/FLT-1, VEGFR-2/FLK-1/KDR, VEGFR-3/FLT-4, FLT-3/FLK-2, CSF-1R, FGFR 1-4, CCK4, Trk A-C, MET (e.g., cMet), RON, EPHA 1-8, EPHB 1-6, AXL, MER, TYRO3, TIE, TEK, RYK, DDR 1-2, RET, c-ROS, LTK (leukocyte tyrosine kinase), ALK (anaplastic lymphoma kinase), ROR 1-2, MUSK, AATYK 1-3, Insulin receptor (IR1), and RTK 106.

In some embodiments, the receptor tyrosine kinase is selected from EGFR, HER2, FGFR1, TrkA, TrkB, cKIT, cMet, and IR1.

As used herein, a “subject” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In one embodiment, the subject is a human. In another embodiment, the subject is an experimental animal or animal suitable as a disease model. The terms “subject” and “patient” are used interchangeably herein.

In some embodiments, the activation wavelength is in far-red or near-infrared spectrum. In some embodiments, the activation wavelength is from about 650 nm to about 900 nm (e.g., 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm).

In some embodiments, the activation wavelength is from about 650 nm to about 700 nm (e.g., 650 nm, 655 nm, 660 nm, 665 nm, 670 nm, 675 nm, 680 nm, 685 nm, 690 nm, 695 nm, 700 nm). In some embodiments, the activation wavelength is from about 700 nm to about 780 nm (e.g., 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775 nm, 780 nm).a

In some embodiments, illumination or light pulses can have a duration for any of about 1 second (sec), about 2.5 sec, about 5 sec, about 7.5 sec, about 10 sec, about 25 sec, about 50 sec, about 75 sec, about 100 sec, about 250 sec, about 500 sec, about 750 sec, about 1000 sec, about 2500 sec, about 5000 sec, about 7500 sec, about 10000 sec, about 25000 sec, about 50000 sec, about 75000 sec, or about 100000 sec inclusive, including any times in between these numbers. In some embodiments, illumination or light pulses can have a light power density of any of about 0.01 mW cm⁻², 0.025 mW cm⁻², 0.05 mW cm⁻², about 0.1 mW cm⁻², about 0.25 mW cm⁻², about 0.5 mW cm⁻², about 0.75 mW cm⁻², about 1 mW cm⁻², about 2.5 mW cm⁻², about 5 mW cm⁻², about 7.5 mW cm⁻², about 10 mW cm⁻², about 12.5 mW cm⁻², about 15 mW cm⁻², about 17.5 mW cm⁻², about 20 mW cm⁻², about 25 mW cm⁻², 50 mW cm⁻², 75 mW cm⁻², or about 100 mW cm⁻² inclusive, including any values between these numbers.

In some embodiments, the method may additionally include expanding the cells in a cell culture medium following the step of introducing to the cells a polynucleotide or a vector, as described above.

The term “culturing” or “expanding” refers to maintaining or cultivating cells under conditions in which they can proliferate and avoid senescence. For example, cells may be cultured in media optionally containing one or more growth factors, i.e., a growth factor cocktail. In some embodiments, the cell culture medium is a defined cell culture medium. The cell culture medium may include neoantigen peptides. Stable cell lines may be established to allow for the continued propagation of cells.

The terms “cell,” “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny with the same function or biological activity as screened or selected for in the originally transformed cell are included herein.

Physical methods for introducing a polynucleotide into a host cell include calcium phosphate precipitation, lipofection, particle bombardment, microinjection, electroporation, and the like. Methods for producing cells comprising exogenous vectors and/or nucleic acids are well known in the art. See, for example, Sambrook et al. (2001, Molecular Cloning: A Laboratory Manual, Cold Spring Harbor Laboratory, New York). Chemical means for introducing a polynucleotide into a host cell include colloidal dispersion systems, such as macromolecule complexes, nanocapsules, microspheres, beads, and lipid-based systems, including oil-in-water emulsions, micelles, mixed micelles, and liposomes. An exemplary colloidal system for use as an in vitro and in vivo release vehicle is a liposome (e.g., an artificial membrane vesicle).

In the case in which a non-viral delivery system is used, an exemplary delivery vehicle is a liposome. Lipid formulations can be used for the introduction of nucleic acids into a host cell (in vitro, ex vivo, or in vivo). In one example, the nucleic acid may be associated with a lipid. The nucleic acid associated with a lipid may be encapsulated in the aqueous interior of a liposome, interspersed within the lipid bilayer of a liposome, bound to a liposome via a binding molecule that is associated with both the liposome and the oligonucleotide, entrapped in a liposome, in a complex with a liposome, dispersed in a solution containing a lipid, mixed with a lipid, combined with a lipid, contained as a suspension in a lipid, content or in a complex with a micelle, or associated otherwise with a lipid. The compositions associated with lipids, lipids/DNA or lipids/expression vector are not limited to any particular structure in solution. For example, they can be present in a bilayer structure, as micelles, or with a “collapsed” structure. They can also be simply interspersed in a solution, possibly forming aggregates that are not uniform in size or shape. Lipids are fatty substances that can be natural or synthetic lipids. For example, lipids include fatty droplets that occur naturally in the cytoplasm as well as the class of compounds containing long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; Dicetylphosphate (“DCP”) can be obtained from K & K Laboratories (Plainview, NY); Cholesterol (“Choi”) can be obtained from Calbiochem-Behring; dimyristyl phosphatidylglycerol (“DMPG”) and other lipids can be obtained from Avanti Polar Lipids, Inc. (Birmingham, AL). Lipid stock solutions in chloroform or chloroform/methanol can be stored at about −20° C. Chloroform is used as the sole solvent since it evaporates more easily than methanol. “Liposome” is a generic term that encompasses a variety of unique and multilamellar lipid vehicles formed by the generation of bilayers or closed lipid aggregates. Liposomes can be characterized as having vesicular structures with a bilayer membrane of phospholipids and an internal aqueous medium. Multilamellar liposomes have multiple layers of lipids separated by an aqueous medium. They form spontaneously when phospholipids are suspended in an excess of aqueous solution. The lipid components undergo self-rearrangement before the formation of closed structures and trap dissolved water and solutes between the lipid bilayers (Ghosh et al., 1991 Glycobiology 5:505-10). However, compositions that have different structures in solution than the normal vesicular structure are also included. For example, lipids can assume a micellar structure or simply exist as nonuniform aggregates of lipid molecules. Lipofectamine-nucleic acid complexes are also contemplated.

Regardless of the method used to introduce exogenous nucleic acids into a host cell, the presence of the recombinant DNA sequence in the host cell can be confirmed by a series of tests. Such assays include, for example, “molecular biology” assays well known to those skilled in the art, such as Southern and Northern blot, RT-PCR and PCR; biochemical assays, such as the detection of the presence or absence of a particular peptide, for example, by immunological means (ELISA and Western blot) or by assays described herein to identify agents that are within the scope of the disclosure.

b. Methods of Identifying Receptor Modulators

In another aspect, this disclosure additionally provides a method for identifying a modulator capable of modulating (e.g., inhibiting, activating) an activity or expression level of a receptor. The method comprises: (a) contacting the modulator with a cell disclosed herein; (b) illuminating the cell by a wavelength; (c) measuring the activity or expression level of the receptor in the cell and in a control cell that has not been contacted with the modulator; (d) comparing the activity or expression level of the receptor in the cell to the activity or expression level of the receptor in the control cell; and (e) identifying the modulator as having modulating activity for the receptor if a difference between the activity or expression level of the receptor in the cell and the activity or expression level of the receptor in the control cell is greater or less than a threshold value.

In some embodiments, the method may include culturing or expanding a cell and/or a control cell. The term “culturing” or “expanding” refers to maintaining or cultivating cells under conditions in which they can proliferate and avoid senescence. For example, cells may be cultured in media optionally containing one or more growth factors, i.e., a growth factor cocktail. In some embodiments, the cell culture medium is a defined cell culture medium. The cell culture medium may include neoantigen peptides. Stable cell lines may be established to allow for the continued propagation of cells.

In some embodiments, the receptor is a receptor tyrosine kinase. Non-limiting examples of receptor tyrosine kinases include EGFR (e.g., EGFR/HER1/ErbB1, HER2/Neu/ErbB2, HER3/ErbB3, HER4/ErbB4), INSR (insulin receptor), FGFR1, IGF-IR, IGF-IIIR, IRR (insulin receptor-related receptor), PDGFR (e.g., PDGFRA, PDGFRB), cKIT/SCFR, VEGFR-1/FLT-1, VEGFR-2/FLK-1/KDR, VEGFR-3/FLT-4, FLT-3/FLK-2, CSF-1R, FGFR 1-4, CCK4, Trk A-C, MET (e.g., cMet), RON, EPHA 1-8, EPHB 1-6, AXL, MER, TYRO3, TIE, TEK, RYK, DDR 1-10) 2, RET, c-ROS, LTK (leukocyte tyrosine kinase), ALK (anaplastic lymphoma kinase), ROR 1-2, MUSK, AATYK 1-3, Insulin receptor (IR1), and RTK 106.

In some embodiments, the receptor tyrosine kinase is selected from EGFR, HER2, FGFR1, TrkA, TrkB, cKIT, cMet, and IR1.

In some embodiments, the modulator is an inhibitor or activator. In some embodiments, the modulator may be an agonist, antagonist, peptidomimetic, protein, peptide, nucleic acid, small molecule, or other drug candidate.

The term “reference level” or “reference value,” as used herein, refers to a threshold level, or a level in a control cell or a control subject or a level (e.g., average level, mean level) obtained from a cell population or a subject population (e.g., patient population).

The activity or expression level of a gene may be measured by determining or estimating a protein level or mRNA level encoded by the gene. Methods for determining or estimating a protein level or mRNA level are well known in the art. For example, the protein level (e.g., protein expression level) of Btnl2 can be determined by SDS-PAGE, Western blot, or an immunoassay (e.g., immunoblotting assay, immunoprecipitation assay). The mRNA level may be determined by RT-PCR.

Measuring the levels of the gene can be performed by assaying the proteins themselves (by Western blotting, ELISA, RIA, and other techniques known to one skilled in the art), by assaying the mRNA encoding these proteins (such as quantitative PCR, Northern blotting, RNAse protection assay, RNA dot-blotting, and other techniques known to one skilled in the art), or by assaying the activity of the regulatory elements of the gene. For example, the activity of regulatory elements can be assessed by reporter constructs consisting of DNA segments from the promoter, enhancer, and/or intronic elements coupled to cDNAs encoding reporters (such as luciferase, beta-galactosidase, green fluorescent protein, or other reporting genes that can be easily assayed). These reporter constructs can be transfected into cells, either stably or transiently.

c. Other Uses of the Fusion Constructs

The disclosed fusion constructs can have various applications. For example, eDr-RTKs can be used in cultured cells for high-throughput screening of drugs that are either activate or inhibit the downstream signaling of specific pathways regulated by particular RTK. Importantly, because FR-light has no crosstalk with light-excited GFP-like reporters and biosensors, light-activation and readout (e.g., relocalization of GFP-fused transcription factor) can be done optically, thus allowing it applications in various applications.

In addition, FR-light activated eDr-RTKs can be used for non-invasive reversible control of RTK signaling in numerous animal models of human diseases with spatiotemporal precision. As demonstrated in this disclosure, eDr-TrkB can be used for optical regulation of immediate early genes and sleep in mice by focused light-activation of a particular location of the cortex.

Also, eDr-RTKs can be used as an alternative to growth factors and insulin injections, especially in immunologically privileged tissues, such as eye, tumor, or the brain. For example, it can be used as an alternative for the optogenetic tools, such as bacterial channelrhodopsin, which was FDA-approved as gene therapy for vision restoration in patients with retinal degeneration. In this therapy, channelrhodopsin DNA or mRNA is delivered to human retinal ganglion cells using AAV or polymer/lipid nanoparticles, respectively.

Additionally, eDr-TrkA can be used for therapy of Alzheimer's disease. Nerve growth factor (NGF) is a natural ligand for TrkA and a therapeutic candidate for Alzheimer's disease because of its protective action of the cholinergic neurons in the brain, but diffusion of the NGF to the peripheral nervous system causes side effects, such as back pain. Moreover, NGF activates not only pro-survival TrkA, but also death receptor p75NTR. Expressing or light-activating eDr-TrkA specifically in cholinergic neurons prevents the off-target effects.

### C. Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

In some embodiments, the polynucleotide may include a codon-optimized sequence. For example, the nucleotide sequence encoding the light-responsive polypeptide variant/fragment may be codon-optimized for expression in a eukaryote or eukaryotic cell. In some embodiments, the codon-optimized light-responsive polypeptide variant/fragment is codon-optimized for operability in a eukaryotic cell or organism, e.g., a yeast cell, or a mammalian cell or organism, including a mouse cell, a rat cell, and a human cell or non-human eukaryote organism.

Generally, codon optimization refers to a process of modifying a nucleic acid sequence to enhance expression in the host cells by substituting at least one codon of the native sequence with codons that are more frequently or most frequently used in the genes of that host cell while maintaining the native amino acid sequence. Various species exhibit a particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. Nucl. Acids Res. 28:292 (2000). Computer algorithms for codon optimizing a particular sequence for expression in a particular host cell are also available, such as Gene Forge (Aptagen; Jacobus, Pa.). As to codon usage in yeast, reference is made to the online Yeast Genome database available at http://www.yeastgenome.org/community/ codonusage.shtml, or Codon selection in yeast, Bennetzen and Hall, J Biol Chem. 1982 Mar. 25; 257 (6): 3026-31. As to codon usage in plants including algae, reference is made to Codon usage in higher plants, green algae, and cyanobacteria, Campbell and Gowri, Plant Physiol. 1990 January; 92 (1): 1-11.; as well as Codon usage in plant genes, Murray et al., Nucleic Acids Res. 1989 Jan. 25; 17 (2): 477-98; or Selection on the codon bias of chloroplast and cyanelle genes in different plant and algal lineages, Morton B R, J Mol Evol. 1998 April; 46 (4): 449-59.

As used herein, the term “variant” refers to a first molecule that is related to a second molecule (also termed a “parent” molecule). The variant molecule can be derived from, isolated from, based on or homologous to the parent molecule. The term variant can be used to describe either polynucleotides or polypeptides.

A variant polypeptide can have an entire amino acid sequence identity with the original parent polypeptide or can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence. Polypeptide variants include polypeptides comprising the entire parent polypeptide and further comprising additional fused amino acid sequences. Polypeptide variants also include polypeptides that are portions or subsequences of the parent polypeptide. For example, unique subsequences (e.g., as determined by standard sequence comparison and alignment techniques) of the polypeptides disclosed herein are also encompassed by this disclosure. Polypeptide variants may include polypeptides that contain minor, trivial, or inconsequential changes to the parent amino acid sequence. For example, minor, trivial, or inconsequential changes include amino acid changes (including substitutions, deletions, and insertions) that have little or no impact on the biological activity of the polypeptide and yield functionally identical polypeptides, including additions of non-functional peptide sequence. In other aspects, the variant polypeptides change the biological activity of the parent molecule. One skilled in the art will appreciate that many variants of the disclosed polypeptides are encompassed by this disclosure. Polynucleotide or polypeptide variants can include variant molecules that alter, add or delete a small percentage of the nucleotide or amino acid positions, for example, typically less than about 10%, less than about 5%, less than 4%, less than 2% or less than 1%.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

A peptide or polypeptide “fragment” as used herein refers to a less than full-length peptide, polypeptide or protein. For example, a peptide or polypeptide fragment can have at least about 3, at least about 4, at least about 5, at least about 10, at least about 20, at least about 30, at least about amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length.

As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid 30 residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine) includes one or more conservative modifications. The Cas protein with one or more conservative modifications may retain the desired functional properties, which can be tested using the functional assays known in the art. As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine).

As used herein, the percent homology between two amino acid or nucleic acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid or nucleic acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

Additionally or alternatively, amino acid or nucleic acid sequences can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the disclosed polypeptides. To obtain gapped alignments for comparison purposes, Gapped BLAST can be utilized as described in Altschul et al. (1997) Nucleic Acids Res. 25 (17): 3389-3402. When utilizing BLAST and Gapped BLAST programs, the default parameters of the respective programs (e.g., XBLAST and NBLAST) can be used. (See www.ncbi.nlm.nih.gov).

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

In some embodiments, the nucleotide sequence is operably linked to a promoter. The term “operably linked” refers to a functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions in the same reading frame.

As used herein, the term “recombinant” refers to a cell, microorganism, nucleic acid molecule or vector that has been modified by the introduction of an exogenous nucleic acid molecule or has controlled expression of an endogenous nucleic acid molecule or gene. Deregulated or altered to be constitutively altered, such alterations or modifications can be introduced by genetic engineering. Genetic alteration includes, for example, modification by introducing a nucleic acid molecule encoding one or more proteins or enzymes (which may include an expression control element such as a promoter), or addition, deletion, substitution of another nucleic acid molecule. Or other functional disruption of, or functional addition to, the genetic material of the cell. Exemplary modifications include modifications in the coding region of a heterologous or homologous polypeptide derived from the reference or parent molecule or a functional fragment thereof.

The term “chimeric” or “heterologous” refers to two components that are defined by structures derived from different sources or progenitor sequences. For example, where “heterologous” is used in the context of a chimeric polypeptide, the chimeric polypeptide can include operably linked amino acid sequences that can be derived from different polypeptides of different phylogenic groupings.

As used herein, the term “pharmaceutical composition” refers to a mixture of at least one compound useful within the disclosure with other chemical components, such as carriers, stabilizers, diluents, dispersing agents, suspending agents, thickening agents, and/or excipients. The pharmaceutical composition facilitates administration of the compound to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound and are physiologically acceptable to the subject. Supplementary active compounds may also be incorporated into the compositions.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### D. Examples

### Example 1

This example describes the materials and methods used in the subsequent EXAMPLE(S).

Molecular cloning. The cDNAs encoding various human RTKs were PCR-amplified from the total HeLa cDNA prepared using a ProtoScriptII First Strand cDNA Synthesis Kit (NEB). Human RNA was purified from HeLa cells using a NucleoSpin RNA purification kit (Macherey-Nagel). The Igκ chain leader amino acid sequence METDTLLLWVLLLWVPGSTGD (SEQ ID NO: 53) was introduced to the N-terminus of DrBphP-PCM and mCherry-DrBphP-PCM from the previously described Myr-Dr-hel4-TrkA plasmid (Leopold, A. V., et al. Nat Commun 10, 1129 (2019)) using reverse PCR, thus replacing the Myr signal with the Igκ secretion sequence, The resultant Igκ-DrBphP-PCM and Igκ-mCherry-DrBphP-PCM were inserted via NheI/XhoI sites into the pcDNA3.1+ plasmid. The EGFR and HER2 parts consisting of the transmembrane and cytoplasmic domains were cloned at the C-terminus of the DrBphP-PCM via XhoI/XbaI sites. The other opto-RTK constructs were designed similarly. The amino acid sequences of the Igκ-mCherry-DrBphP-PCM-EGFR and Igκ-mCherry-DrBphP-PCM-HER2 prototype constructs are shown in Table 1.

Mammalian cell culture and transfection. PC6-3 cells were obtained from ATCC and cultured in RPMI-1640 medium supplemented with 10% horse serum (HS) and 5% fetal bovine serum (FBS) (both from Biowest). HEK293 cells were obtained from ATCC and cultured in DMEM medium supplemented with 10% FBS and penicillin-streptomycin mixture (Gibco) at 37° C. For luciferase assay, 20,000 PC6-3 cells were seeded in 0.5 ml medium per well in 24-well plates and transfected with 1 μg of eDrRTK, and pFr-Luc and pFA-Elk-1 plasmids from the PathDetect trans-reporting system (Agilent) in mass ratios of 5:100:5, 1:100:5 and 1:200:10. Before transfection, a Turbofect reagent (Thermo Fisher Scientific) was added to plasmid DNA at a volume-to-mass ratio of 2:1. After 6 h of incubation, the transfection medium was changed to a serum-starving medium (RPMI with 2.5% HS and 25 μM BV). For induction of Elk-1 dependent luciferase transcription in Hela cells without BV, the culture medium was changed to DMEM with 10% FBS 6 h after transfection. Cells then were kept for 24 h under either 660 nm (0.5 mW cm⁻²) or 780 nm (0.5 mW cm⁻²) light. For Western blot, HEK293 cells were seeded in 6-well plates and co-transfected with 4 μg of total DNA per well with opto-RTK and pcDNA3.1+ plasmids in a mass ratio of 1:5. 6 h after transfection, and the medium was changed to DMEM with 1% FBS. For Ca²⁺ measurements, HEK293 cells plated on Nunc glass-bottom dishes were co-transfected with 2.5 μg of total DNA plasmids encoding eDrRTKs and GCAMP6m in a mass ratio of 1:1 using Turbofect reagent.

Bioluminescence assay. PC6-3 cells were transfected as described above and kept in darkness or upon (0.5 mW cm⁻²) illumination for 24 h and were then lysed in 100 μl of lysis buffer (20 mM Tris-HCl, 10% glycerol, 0.1% Triton X-100, 1 mM PMSF, 0.1% β-mercaptoethanol, pH 8.0) for 10 min at room temperature on a swinging platform shaker. Luciferase assay was performed in 96-well half-area white plates (Costar) by mixing 10 μl of cell lysate with 20 μl of firefly luciferase substrate (Nanolight Technology). Bioluminescence was measured immediately with a Victor X3 multilabel plate reader (Perkin Elmer), and data were analyzed with an OriginPro v. 8.6 software.

Immunoblotting and antibodies. For immunoblotting of EGFR, HER2, IR1, TrkA, TrkB, FGFR1, ERK, Akt, and PLCγ, the HEK293 cells were plated in 6-well plates and transfected at 90% confluence with Lipofectamine 3000 reagent (ThermoFisher Scientific). For detection of phosphorylated receptors and downstream signaling molecules, a pcDNA3.1+ and eDrRTKs were mixed with a pcDNA3.1+ at a mass ratio of 1:5. Lipofectamine/DNA complexes were formed for 15 min and added to the wells drop-wise. After 6 h the medium was changed to DMEM medium with 1% FBS and 25 μM BV. Then cells were kept in darkness overnight (for an additional 12 h) and then were activated with the 660 nm light (0.5 mW cm⁻²) for 1 and 10 min. Induced and non-induced cells were put on ice, washed with ice-cold PBS, and lysed in 300 μl of ice-cold RIPA buffer (ThermoFisher Scientific) supplemented with phosphatase and protease inhibitors (ThermoFisher Scientific). Cell lysis was performed for 5 min. Lysates were centrifuged at 12,000 rpm for 20 min in an Eppendorf centrifuge at 4° C. For phospho-ERK detection, 20 μl of lysate per lane was loaded to 10% gel. For detection of phosphorylated eDrRTKs and their downstream signaling targets, 10 μl of lysate per lane was loaded to 10% gel. Proteins were separated by SDS-PAGE and transferred to nitrocellulose membranes. Membranes were blocked in a 5% solution of non-fat dry milk overnight. Then the membranes were incubated overnight at +4° C. with antibodies against phosphorylated ERK (1:2000, #2234, Cell Signaling Technology), phosphorylated EGFR (1:1000, #2234, Cell Signaling Technology), phosphorylated FGFR1 (1:1000, #9740, Cell Signaling Technology), phosphorylated TrkA/TrkB (1:1000, #4619S, Cell Signaling Technology), phosphorylated IR1 (#3023S), phosphorylated Akt phosphorylated at Thr308 (1:1000, #9275, Cell Signaling Technology) or phosphorylated PLCγ (1:1000, #2821, Cell Signaling Technology) diluted in 5% solution of non-fat dry milk. Membranes were washed 4 times by TBS with 0.5% Tween and incubated with goat anti-rabbit HRP conjugate (1:2000) for 2 h at room temperature and then washed by TBS with 0.5% Tween. Bioluminescence was detected using a Clarity Western ECL Substrate (BioRad). Images were taken with a ChemiDoc imaging system. For the detection of the non-phosphorylated eDrRTKs and their downstream signaling targets, the same samples were run in parallel, transferred to nitrocellulose membranes, and stained separately with antibodies against non-phosphorylated EGFR (#2232, Cell Signaling Technology) and FGFR-1 (#3471, Cell Signaling Technology), TrkA (#2505), IR1 (#3020), ERK (#4695), Akt (#4685), PLCγ (#2822) all from Cell Signaling Technology and TrkB (ab18987) from Abcam. GAPDH was used as a loading control. Antibodies against GAPDH (sc-47724, Santa-Cruz) were used in 1:1000 dilution.

Activation of calcium signaling. For analysis of Ca²⁺⁺ signaling, HEK293 cells were co-transfected with the GCaMP6m and eDrRTKs plasmids in a mass ratio of 1:5. 24 h after transfection, cells were serum-starved using 0.2% FBS in darkness overnight. After starving, the medium was changed to Hank's balanced salt solution with 0.2 mM Ca²⁺. The following timelapse Ca²⁺ imaging steps were applied: (i) 3 s 780 nm and 100 ms 480 nm cycles for the first 50 s, (ii) 100 ms 480 nm and simultaneously 660 nm 25 s (0.5 mW cm⁻²) for the next 25 s, and (iii) 3 s 780 nm and 100 ms 480 nm cycles for the rest of timelapse imaging. Cellular Ca² intensity changes were analyzed using ImageJ and OriginPro software. For graphs, the intensity data were calculated as ΔF/F₀ and normalized to 0:1.

Activation of ERK signaling in isolated neurons. For analysis of eDrTrkB downstream signaling in cortical neurons was analyzed. Rat cortical neurons were prepared by a Neuronal Cell Culture unit of the University of Helsinki from the late embryonic stage (E17-18) rat embryos. Animal work was performed under the ethical guidelines of the European Convention and regulations of the Ethics Committee for Animal Research of the University of Helsinki. Neurons were plated in a 96-well white glass-bottom plate (Corning) at a plating density of 20,000 cells per well, grown for 7 days in vitro, and transduced with an AAV9 encoding mCherry-DrTrkB under CAMKII promoter for 24 h. After that, the transduction medium was changed to a medium with 5 μM of BV, and dishes with neurons were kept in the darkness overnight. After that, dishes with neurons were induced for 1 and 10 minutes correspondingly and fixed with the help of paraformaldehyde 4% solution. Fixed neurons were then stained for ERK with the help of primary anti-ERK antibodies (#4695, Cell Signaling) and secondary Alexa488 conjugated antibodies. Imaging of neurons was performed using an Opera Phenix High-Content microscope (Perkin Elmer) equipped with a 60× water-immersion objective.

Activation of cFos expression in neuronal N2a cell line. The N2a cells were cultured in DMEM with 10% FBS and then transfected with eDrTrkB plasmid and pcDNA3.1+ plasmid in a 1:5 ratio. 6 h after transfection, the medium was changed to the serum-starving medium with 5 mM BV and 1% FBS, and then cells were kept in darkness for 24 h or activated for 1, 12, or 24 h with 660 nm light (0.5 mW cm⁻²). Cell lysates collected at these different time points were collected and analyzed by Western blot. The expression of cFos was analyzed with anti-Fos antibodies (sc-253, Santa Cruz Biotechnology).

Animals. C57BL/6J mice were obtained from Jackson Laboratories (stock no. 000664) or bred in-house. Mice were housed at 70° F. with a 12 h light/dark cycle (7:00 AM-7:00 PM) with food and water ad libitum. All experiments with animals conformed to the US Veterans Administration, Harvard University, and the US National Institutes of Health guidelines.

Viral vector. AAV vectors were produced at the Boston Children's Hospital Viral Core. Stereotaxic injection of mCherry expressing AAV injected was AAV2/9-CW3SL-E-mCherry-DrTrkB/AAV2/9-shMBVR-HO1-Fd-Fnr-CW3SL (1:1).

Stereotaxic surgery and viral injection. Mice were deeply anesthetized with isoflurane (1-3%) and fixed to a stereotaxic frame. Skin over the skull is incised and retracted to expose the skull. A hole is drilled on the skull for viral injection into the cortex. EEG (electroencephalogram) electrode was screwed into the skull above frontal cortex (AP=1.7; ML=±1), a reference electrode over cerebellum, EMG (electromyography) electrodes were placed in the nuchal muscle. The electrodes were then connected to a plug (PlasticsOne) and fixed to the skull using dental cement. Viral injections were performed using a 1-μL Hamilton syringe (7000 series; Hamilton) connected to 30-gauge tubing filled with mineral oil connected to 35G Blunt NanoFil needle (catalog no. NF35BL-2; WPI) for targeted injection of 0.5 μL into the unilateral cortex [AP=1.0, medial-lateral (ML)=2.0, ventral (V)=0.8]. A 1-μL virus aliquot was backloaded via the tip of the injector needle, and the syringe was placed in a stereotaxic horizontal holder. The injector needle was inserted through the dura and placed above the target cortex region, and 0.5 μL of the virus aliquot was slowly injected for 10 min. The injector needle was left in place for an additional 10 min to allow virus diffusion into the brain and to avoid backflow along the cannula/needle tract. Once viral injection was complete, the injector needle was removed, and the red-light LED (629 nm; Lumileds) and infrared LED (810 nm; Vishay Semiconductors) were fastened on the top of the skull with clear acrylic cement above the cerebral cortex injected with AAV. Following the injection and electrodes/LED fixed to the skull by dental cement, the scalp incision was sutured closed, and mice were allowed to recover.

In vivo EEG/EMG recordings. The mouse was tethered to the F20-EET wireless transmitter (Data Sciences International) on a swivel-mounted system (Neurotargeting Systems), allowing the mice to move freely in their cage as previously described (Zielinski, M. R., et al. J Neurosci Methods 216, 79-86 (2013)). The cage was positioned on top of the receiver plate that functioned to detect potential data from FM signals from the transmitter to a data exchange matrix (Data Sciences International). The signals were transferred to a computer using the Dataquest A.R.T system. EEG and EMG signals were amplified, analog-to-digital-converted, and stored at 500 Hz. After four weeks of postsurgical recovery and two days of adaptation to the recording chamber, EEG and EMG were recorded via telemetry using DQ ART 4.1 software (Data Sciences International). Optogenetic stimulation was performed for 24 h by generating light flashes at the following pattern: 0.5 min light on and 3 min light off repeated during the 24 h period. Continuous EEG/EMG recordings were performed before, during, and after light stimulation of cortical neurons.

Light stimulation of the cortex. Light stimulation was performed unilaterally to stimulate cortical neurons transfected with AAVs using a 629 nm or 810 nm LED light source implanted on the skull. Software programmed ATTINY85 microcontroller-generated pulses were used to drive the LED light stimulation. The light stimulation of DrTrkB-transduced cortical cells was delivered every 3 min at 30 s duration (30 s light on/3 min light off) for 24 h period beginning at ZT-9 while the sleep/wake recording continued. Baseline recordings were done for 24 h a day before the light stimulation. Immediately after completion of light stimulation, the mice were perfused with PBS and 10% (vol/vol, HT5011, Sigma) formalin solution for immunohistochemistry (see below).

Immunostaining. The animals were transcardially perfused following our previously performed procedures (Moriarty, S. R. et al. Proc Natl Acad Sci USA 110, 20272-20277 (2013)). The brains were kept in 10% formalin overnight and then incubated in 30% sucrose for at least 1 day before slicing. Double staining of RFP and cFos were performed on 40 μm-thick brain slices to verify the AAV transduction and to detect the neuronal activity, respectively. The primary antibodies were rabbit anti-RFP (1:2000) (600-401-379-RTU, Rockland Immunochemicals) and rabbit anti-cFos (1:1000) (sc-253, Santa Cruz Biotechnology). The secondary antibody was anti-Rbt-Biotin (1:500) (AP132B, Chemicon International). The ABC (PK-6100, Vector Laboratories Inc., Burlingame, CA) and DAB (SK-4100, Vector Laboratories) staining was performed to visualize the cFos labeled cells (black color in the nuclei). The ABC-AP and Vector Red AP Substrate (AK-5000, Vector Laboratories) was used to visualize the RFP-expressing cells (red color in the cytoplasm). Double staining using anti-NeuN antibody (1:500; MAB377, Millipore) and biotinylated anti-GFP antibody (1:1000; BA-1000, Vector Laboratories) was performed to verify the AAV transduction in neurons. After the slices were mounted onto the glass slides with VectaMount (Vector Laboratories), the Olympus BX51 microscope and the stereo investigator system (MBF Bioscience) were used to observe the neurons with RFP and/or cFos expression.

Sleep data analysis. Data file saved by DSI software was imported into the SleepSign program for further scoring sleep states into 5 sec epochs. Sleep scoring during 24 h recording for light stimulation was compared to control/mock (no LED light stimulation) for both 629 nm and 810 nm light sources. Behavioral states were scored as one of the following: (1) Wake; active behavior accompanied by desynchronized EEG (low in amplitude), and tonic/phasic motor activity evident in the EMG signal; (2) NREM sleep; more synchronized EEG, higher in amplitude, with particularly notable power in the delta (0.5-4 Hz) band, and low motor activity (EMG); and (3) REM sleep; small amplitude EEG, particularly notable power in the theta (4-9 Hz) band, and phasic motor activity (EMG). Compared to wake, EEG power during REM sleep was significantly reduced in delta frequencies (0.5-4 Hz) and increased in the range of theta activity (4-9 Hz). The measures included calculating the amount and % time spent in the wake, NREM sleep, or REM sleep, as well as number and duration of bouts, during 24 h recording during light stimulation compared to control/mock (no light stimulation).

EEG data analysis. To analyze the data, multi-taper spectral analysis (Prerau, M. J., et al. Physiology (Bethesda) 32, 60-92 (2017)); Chronux Toolbox, Chronux.org) was performed on 24 h raw EEG data, using custom scripts written for MATLAB (R2016a-2018b, Math Works). The power values in the time-frequency spectra from each investigation (NREM sleep or wake or REM sleep states) were then normalized to the mean power in each frequency 1 h bin across the 24 h recording in mock/baseline and during stimulation recordings. Mock stimulation (baseline) experiments were performed in the same manner as those with light stimulation, but with the LED turned-OFF. These normalized spectra of total power for each investigation (NREM sleep or wake or REM sleep states) were then averaged across all mice. For statistical analysis of this data, power across the frequency band of interest (slow-wave activity, 0.5-1.5 Hz and delta, 0.5-4 Hz) for each 24 h EEG segment for NREM, REM sleep, and wake values compared between mock/baseline recording vs. stimulation recording.

Statistics. For light stimulation experiments, comparisons between mock/baseline and light stimulation were performed using paired t-tests. Statistical analysis was performed using SPSS software (release 11.5), and differences were determined to be significant when P<0.05. All averaged data are shown as means±standard error. For optogenetic stimulation studies analyzing cortical EEG delta power during behavioral states in mice exposed to light stimulation (629 nm or 810 nm), the data were compared to mock stimulation (No-light)/baseline recording using paired t-tests. The total mean EEG band power in <1.5 Hz (low delta) and 0.5-4 Hz (delta) during Wake, NREM sleep, and REM sleep over 24 h optogenetic stimulation were compared to the mock/baseline recording period from all mice in the group, and differences were determined to be significant when P<0.05. Due to the inequality of variances found between the two conditions (spectral powers across mock stimulation/baseline and optogenetic stimulation periods), data were analyzed using the Wilcoxon Signed-Rank test.

### Example 2

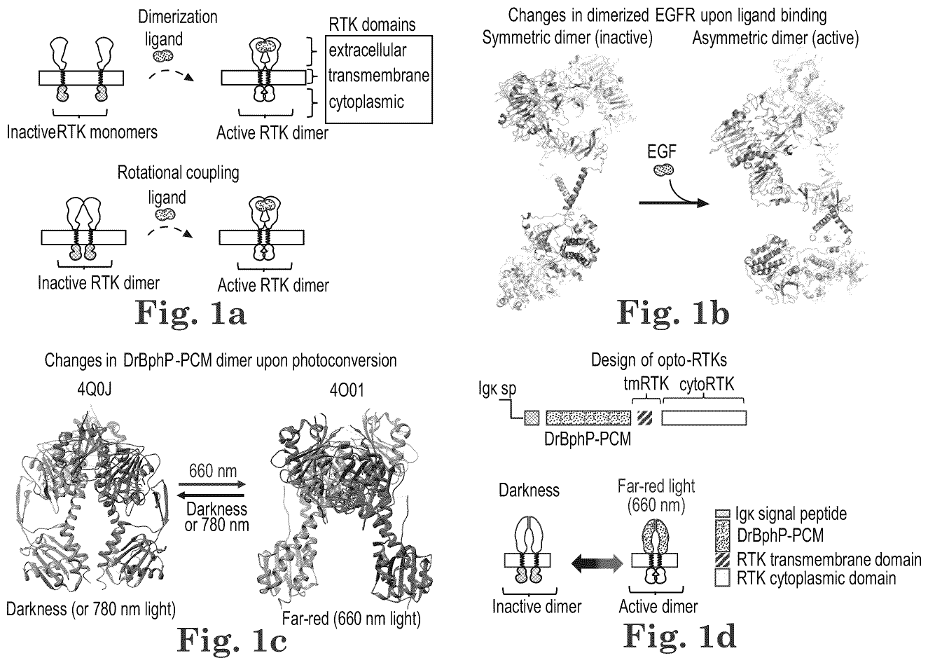

To develop opto-RTKs inactive in darkness and activated (rather than inactivated) with light and to minimize the distance between the catalytic domain and plasma membrane, DrBphP-PCM was targeted to the cell surface by fusing N-terminally with Igκ signal peptide and C-terminally with RTK intracellular domains via their transmembrane helices (FIG. 1d). It was hypothesized that the transmembrane helices, which transmit conformational changes from the extracellular RTK domain to the cytoplasmic catalytic domain would similarly transmit activation signals from the extracellular targeted DrBphP-PCM. EGFR (epidermal growth factor receptor) and HER2 (human epidermal growth factor receptor 2) RTKs were first tested by engineering this type of chimeric integral membrane proteins. The resultant EGFR- and HER2-derived opto-RTKs were inactive in darkness and activated with FR light. This protein design was then extended to engineer opto-RTKs from several other RTK families. The performance of these opto-RTKs was characterized by Western blot, activation of ERK1/2-dependent gene transcription, PLCγ signaling, and Ca²⁺ fluxes. The performance of one of the new constructs was next validated in living mice. One based on TrkB (tropomyosin receptor kinase B) was chosen because of its important role in the development, memory formation, regulation of sleep, neuroprotection, and synaptogenesis (Gupta, V. K., et al. Int J Mol Sci 14, 10122-10142 (2013)). It was found that its stimulation with FR light in the cerebral cortex induced neuronal activity and affected sleep patterns, indicating advances of DrBphP-based opto-RTKs for in vivo applications.

### Engineering of Opto-RTK Prototypes.

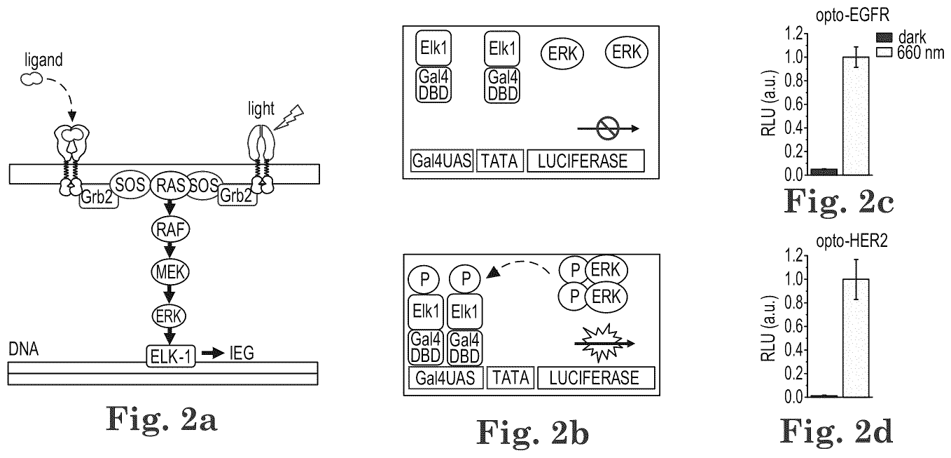

First, the DrBphP-PCM fusions consisting of the N-terminally fused Igκ secretion signal peptide and the C-terminally fused transmembrane domains (tm) of human EGFR or HER2 followed by their cytoplasmic domains (cyto) were constructed, designated Igκ-DrBphP-PCM-tmEGFR-cytoEGFR and Igκ-DrBphP-PCM-tmHER2-cytoHER2 (FIG. 1d). EGFR (ErbB-1 or HER1) and HER2 (ErbB-2) belong to the EGFR RTK family, which plays an important role in the regulation of cell growth, proliferation, and tumorigenesis. EGFR is activated by several ligands, including EGF, transforming factor-alpha (TGFA), amphiregulin, and others, while HER2 does not have a ligand and relies on heterodimerization with other family members or homodimerization when overexpressed. Both EGFR and HER2 activate ERK1/2 cascade and subsequent expression of immediate early genes (IEGs) by phosphorylating several transcription factors, including Elk-1 (FIG. 2a).

Next, the ability of these chimeras to activate light-control ERK1/2-dependent luciferase expression was tested using the PathDetect trans-reporting system (Agilent). In this system, ERK1/2 phosphorylates transcription factor Elk-1 fused to the yeast GAL4 DNA-binding domain (DBD: residues 1-147). A pFR-Luc reporter plasmid encodes firefly luciferase under a synthetic promoter, containing five upstream activating sites (5×UAS) of GAL4. Elk-1 phosphorylation by ERK1/2 leads to the GAL4 dimerization. The dimeric GAL4-DBD binds the 5×UAS sequence in pFR-Luc and activates luciferase expression, reporting activation of the ERK1/2 pathway (FIG. 2b). This assay allows evaluating the photoactivation contrast of opto-RTKs (Leopold, A. V., et al. J Mol Biol 432, 3749-3760 (2020); Leopold, A. V., et al. Nat Commun 10, 1129 (2019)).

Upon action of FR light, in PC6 cells that have been used to characterize opto-RTKs, the resultant Igκ-DrBphP-PCM-tmEGFR-cytoEGFR and Igκ-DrBphP-PCM-tmHER2-cytoHER2 activated the luciferase expression by several tens-fold (FIG. 2c). The several-fold stronger activation by opto-HER2 than that by opto-EGFR was consistent with the literature data on the higher Elk-1 expression during HER2 signaling.

### Optimization of Opto-EGFR and Opto-HER2 Prototypes.

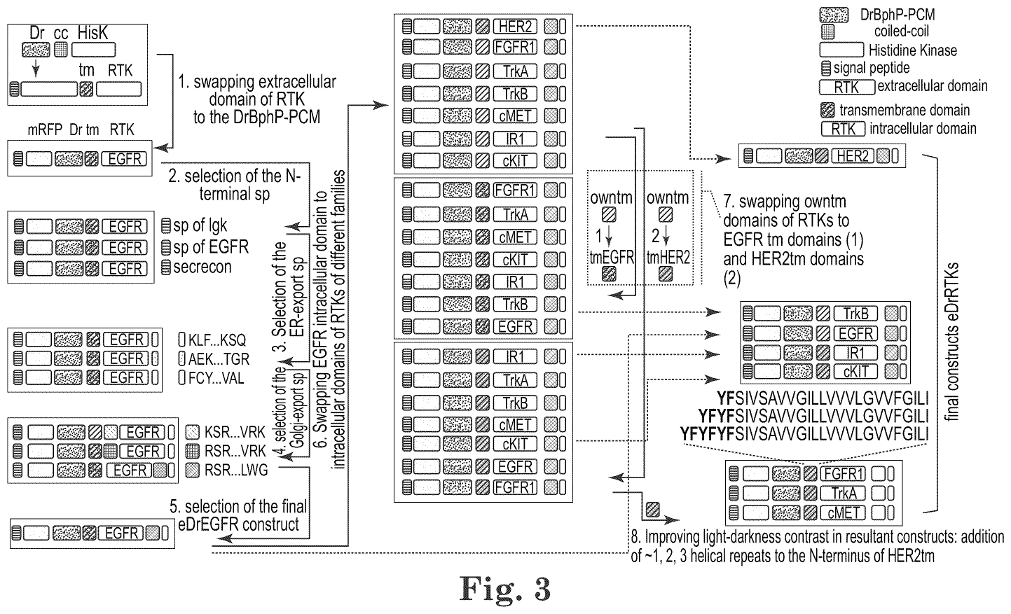

To improve the plasma membrane localization of the opto-EGFR and opto-HER2 prototypes, two extracellular export signals were first tested by swapping the Igκ secretion signal peptide with Secrecon (Barash, S., et al. Biochem Biophys Res Commun 294, 835-842 (2002)) or own signal peptide of EGFR (FIG. 3). However, neither of them improved the plasma membrane localization of these fusions constructs. Next, several endoplasmatic reticulum (ER) export signals were tested, such as two di-acidic C-terminal export signals ANSFCYENEVAL (SEQ ID NO: 54) and AEKMDIDTGR (SEQ ID NO: 55) and one di-hydrophobic motif KLFYKAQRSIWGKKQ (SEQ ID NO: 56) (Barlowe, C. et al. Trends Cell Biol 13, 295-300 (2003)). Of these ER export motifs, the ANSFCYENEVAL (SEQ ID NO: 57) signal from Kir2.1 channel was choose. To further optimize the plasma membrane location, several Golgi-export sequences (Golgi patch: GP) were next inserted, such as KSRITSEGEYIPLDQIDINV (SEQ ID NO: 58), RSRFVKKDGHCNVQFINV (SEQ ID NO:59), and RSRFVKKD(SAG)₄SYLANEILWG(SAG)₄ (SEQ ID NO: 60) placed C-terminally to the tmRTK or C-terminally to the cytoRTK domains. The insertions between the tmRTK and cytoRTK worsened the opto-RTK signaling while the insertion at the C-terminus of opto-RTKs the latter GP sequence from the Kir2.1 channel (Ma, D. et al. Cell 145, 1102-1115 (2011)) did not intervene the high signal of opto-RTK activation.

These optimized constructs were termed eDrEGFR (enhanced DrBphP-PCM-based EGFR) and eDrHER2 (enhanced DrBphP-PCM-based HER2) and exhibited light-induced luciferase reporter expression ˜30 fold and ˜100-fold, respectively.

### Extending Engineering Approach to Other RTKs.

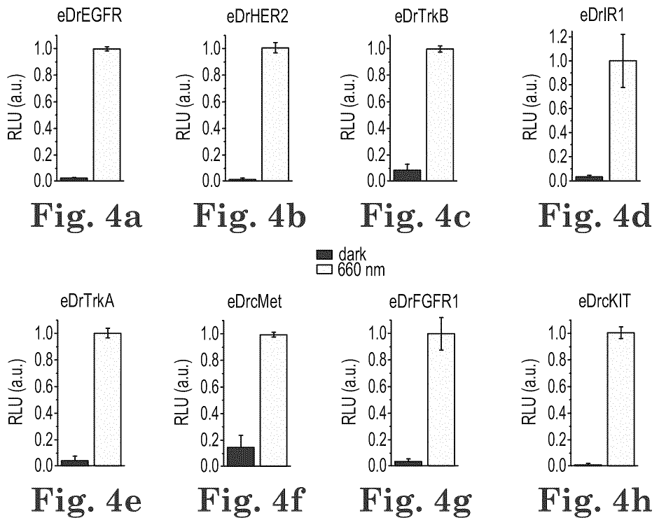

Next, the possibility of applying the same opto-RTK design to six other RTKs was tested, such as TrkA (tropomyosin receptor kinase A), TrkB, IR1 (insulin receptor 1), cKIT (tyrosine-protein kinase KIT), FGFR1 (fibroblast growth factor receptor 1), and cMet (cancer mesenchymal to epithelial transition). For this, the RTK fragments consisting of their transmembrane domain (tmRTK) followed by the cytoplasmic domain (cytoRTK) were inserted between Igκ-DrBphP-PCM and GP-FCYENEV motifs via the XhoI-XbaI restriction sites. Their ability to light-activate ERK1/2-dependent reporter expression in PC6-3 cells was verified. All Igκ-DrBphP-PCM-tmRTK-cytoRTK fusions activated luciferase production upon FR illumination. However, in contrast to eDrEGFR and eDrHER2, the activation contrast (a difference between bioluminescence signals in the light and dark conditions) of these RTK fusions did not exceed ˜3.5-fold. It was hypothesized that transmembrane domains of EGFR (tmEGFR) and HER2 (tmHER2) transmitted the light-induced conformation changes across the plasma membrane more efficiently and, consequently, provided the higher light-activation contrast in eDrEGFR and eDrHER2 (FIGS. 4a-b). Therefore, all endogenous tmRTKs in the other RTK constructs were swapped with tmEGFR or tmHER2 (FIG. 3).

Changing the tmTrkB in the TrkB construct to tmEGFR, and the tmIR1 and tmcKIT in the IR1 and cKIT constructs, respectively, to the tmHER2 improved optogenetic activation to ˜15-40 fold, resulting in opto-RTKs termed eDrTrkB, eDrIR1, and eDrcKIT (FIG. 3). However, in the other three constructs altering their transmembrane domains to tmEGFR and tmHER2 did not lead to substantial improvement in contrast. Since, except for TrkB, alteration of transmembrane domains to tmEGFR resulted in lower activation of the ERK1/2 pathway, the focus was re-directed to constructs with tmHER2.

It was hypothesized that changing the number of turns of the α-helix in tmHER2 could improve the fusion performance. Since the α-helix has 3.6 amino acid residues per turn, 2, 4, and 6 residues were inserted at the N-terminus of tmHER2. To maintain the helicity, hydrophobicity, and correct orientation of tmHER2, the -YF- amino acid repeats were added. Parts of the transmembrane helices located closer to the outer membrane leaflet are enriched in aromatic residues, such as phenylalanine and tyrosine, that likely increase α-helix propensity (Lu, P. et al. Science 359, 1042-1046 (2018)). The N-terminal insertion of the -YF- and -YFYFYF- (SEQ ID NO: 61) sequences in tmHER2 in the TrkA, FGFR1, and cMet constructs decreased the ERK1/2 activation to 1.5-fold, whereas the addition of -YFYF- (SEQ ID NO: 62) residues drastically improved contrast to ˜40-fold in opto-FGFR1 and opto-TrkA and to ˜6-fold in opto-cMet. The resultant opto-RTKs were termed eDrTrkA, eDrFGFR1, and eDrcMet (FIGS. 3 and 4).

Thus, the disclosed protein engineering approach with systematic optimization of initial fusions resulted in 8 eDrRTKs of different RTK families that are inactive in the darkness and reversibly activated by FR light (FIGS. 3 and 4).

Light-Induced Phosphorylation of eDrRTKs and ERK1/2.

The PathDetect trans-reporting system detects RTK activation in a matter of hours. To characterize activation of eDrRTKs on a minute time scale, lysates of HEK293 cells transfected with eDrRTKs using Western blot were studied. The phosphorylation of eDrRTKs and their downstream signaling target ERK1/2 were directly analyzed using specific anti-phospho-RTK antibodies.

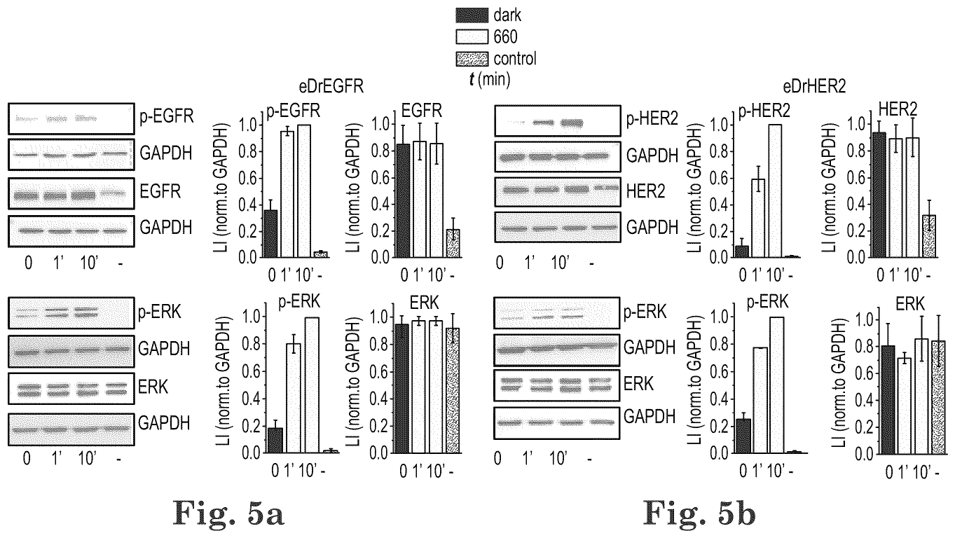

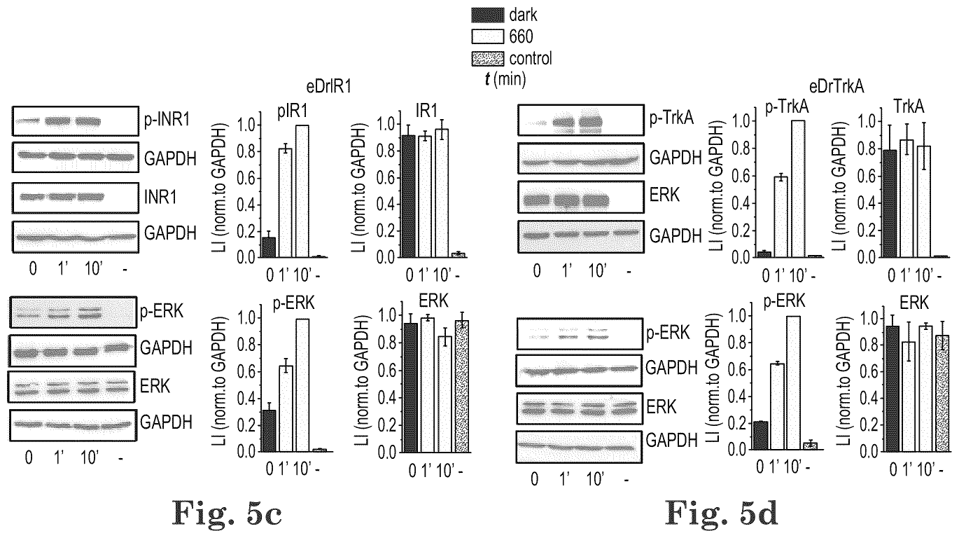

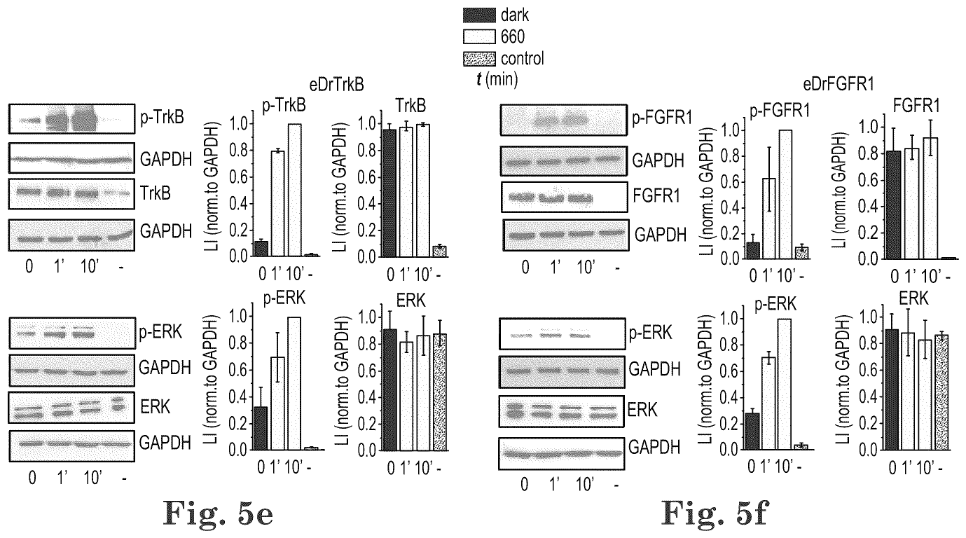

FR illumination of the HEK293 cells for 1 min led to robust phosphorylation of the majority of eDrRTKs, while 10 min illumination caused the maximal ERK1/2 phosphorylation (FIG. 5). These results demonstrated that the eDrRTKs phosphorylation can be non-invasively triggered with FR light in a matter of minutes.

### Reversibility of Light-Activation of PLCγ Pathway and Ca²⁺ Signaling.

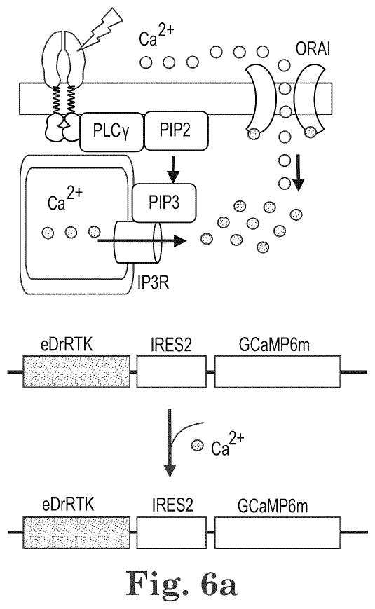

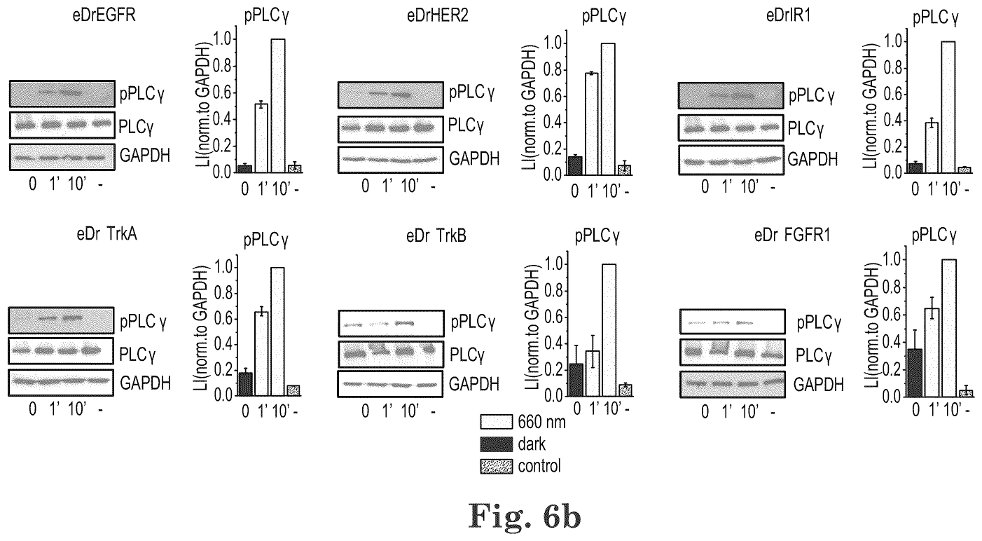

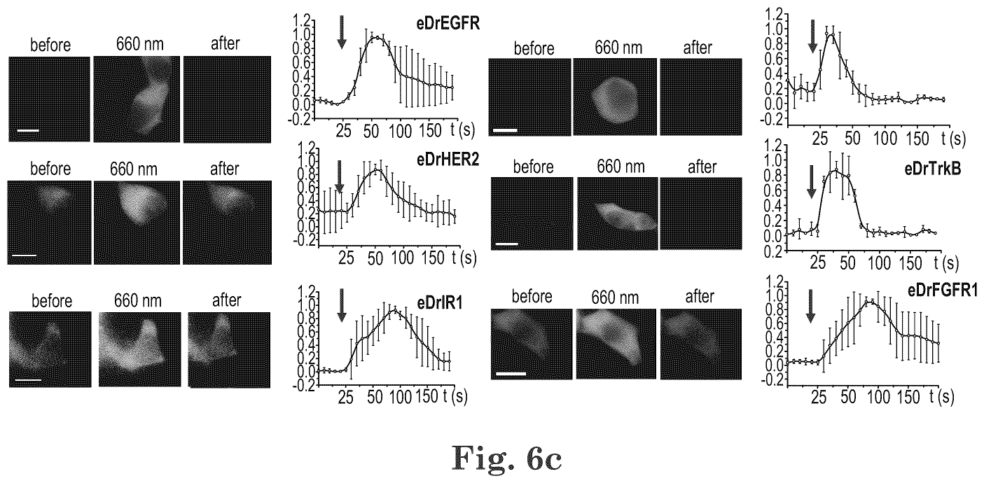

Phospholipase Cy (PLCγ) is activated by all RTKs. PLCγ contains a prototypical Src homology 2 (SH2) containing substrate, which is recruited to one of the phosphorylated RTK molecules and then is phosphorylated by another RTK molecule. Phosphorylated PLCγ translocates to the plasma membrane and catalyzes the hydrolysis of phosphatidylinositol-4,5-biphosphate (PIP2) to diacylglycerol (DAG) and inositol-1,4,5-triphosphate (IP3) (FIG. 6a).

To test whether eDrRTKs can trigger PLCγ signaling, PLCγ phosphorylation was studied in HEK293 cells by Western blot. The eDrRTK-expressing cells were activated with FR light for 1 or 10 min, and the lysate was examined with anti-phospho-PLCγ antibodies. Western blot showed that eDrRTKs activated PLCγ several-fold after 1 min of FR illumination, and the phosphorylation increased in the following 10 min (FIG. 6b).

Because PLCγ signaling is related to induction of cellular Ca²⁺, the possibility of multiplexing eDrRTKs was evaluated with a green fluorescent protein (GFP)-based Ca²⁺ biosensor GCaMP6m (Chen, T. W. et al. Nature 499, 295-300 (2013)). Activation of HEK293 cells co-expressing eDrRTKs and GCaMP6m with 25 s FR light pulse caused up to ˜10-fold increase in GCaMP6m fluorescence, which then decreased after FR light was turned off (FIG. 6c). From the beginning of the FR light pulse, the maximal Ca²⁺ levels were achieved at ˜72 s with half-times of ˜10-15 s. Ca²⁺ transients were readily reversed with 60 s pulse of NIR 780 nm light with half-times of ˜45-55 s.

Notably, imaging of GCaMP6m was spectrally compatible with activation and inactivation light of eDrRTKs, indicating that the DrBphP-PCM-based opto-RTKs allow the crosstalk-free combination with fluorescent proteins and biosensors that are excited in the visible light range.

eDrTrkB-Activated cFos Expression in Cultured Neuronal Cells.

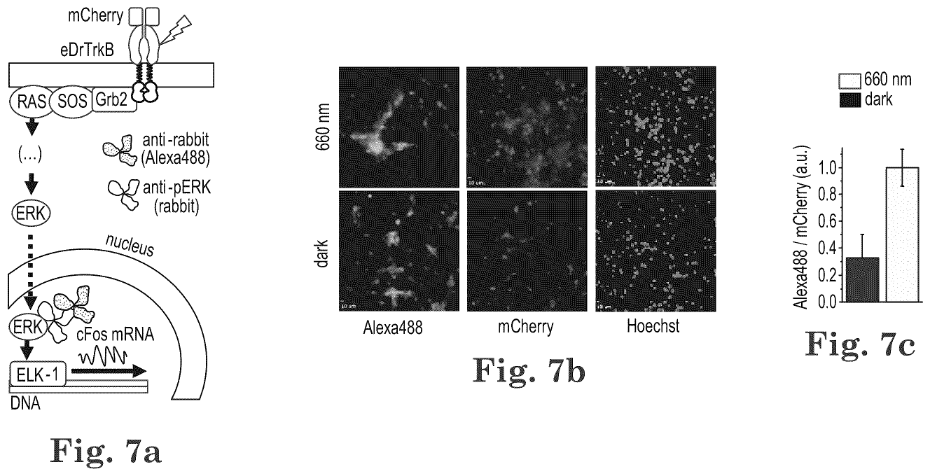

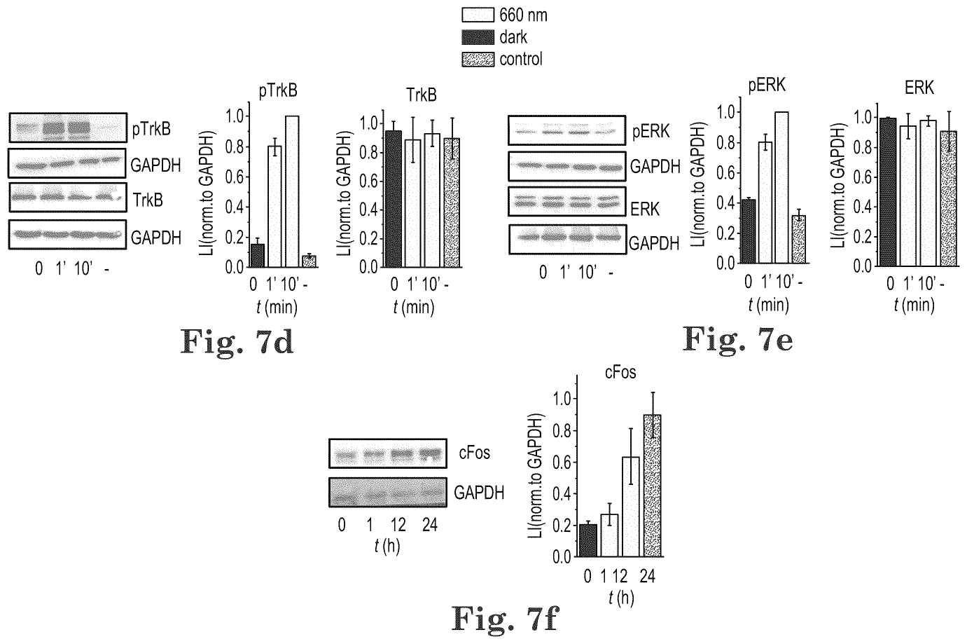

TrkB is a neurotrophin receptor involved in several processes in the nervous system, including sleep, learning, long-term potentiation, and depression. TrkB signaling in neurons leads to activation of ERK1/2 cascade, phosphorylation of transcription factor Elk-1, and translation of cFos mRNA (FIG. 7a).

To evaluate eDrTrkB signaling in isolated rat cortical neurons, the isolated rat cortical neurons were transduced with the adeno-associated virus serotype 9 (AAV9) encoding a mCherry-eDrTrkB construct under a neuron-specific CaMKII promoter. FR illumination of the neurons expressing mCherry-eDrTrkB caused activation of ERK1/2 signaling and translocation to the cell nucleus as revealed by staining with primary rabbit anti-phospho-ERK1/2 and secondary anti-rabbit-Alexa488 Abs (FIG. 7b). A more than 3-fold increase of the phosphorylated ERK1/2 in the nucleus occurred in a matter of minutes (FIG. 7c). Similarly, the FR light induced the several-fold increase of the TrkB and ERK1/2 phosphorylation on a minute time scale in the eDrTrkB-expressing mouse neuroblastoma N2a cells as revealed by Western blot (FIGS. 7d-e).

To evaluate the ability of eDrTrkB to upregulate cFos expression, the N2a cells were transfected with eDrTrkB plasmid and activated it with FR light for 1, 12, and 24 h. FR light upregulated cFos expression already after 1 h and reached ˜5-fold increase after 24 h of the eDrTrkB photoactivation (FIG. 7f).

Effect of eDrTrkB Activation in Neocortex on Sleep/Wake Pattern.

Extensive evidence indicates that the neurotrophin brain-derived neurotrophic factor (BDNF), a primary ligand of TrkB, is a molecular mediator of sleep homeostasis. Animal studies demonstrated that the amount of exploratory behavior during wakefulness predicts both the extent to which BDNF is induced and the extent of the homeostatic delta power response during subsequent sleep. Administration of exogenous BDNF increases sleep time, and gene polymorphisms influencing BDNF secretion in humans are associated with changes of EEG markers of sleep intensity. Therefore, the effect of eDrTrkB activation on sleep amounts and EEG power spectrum was studied.

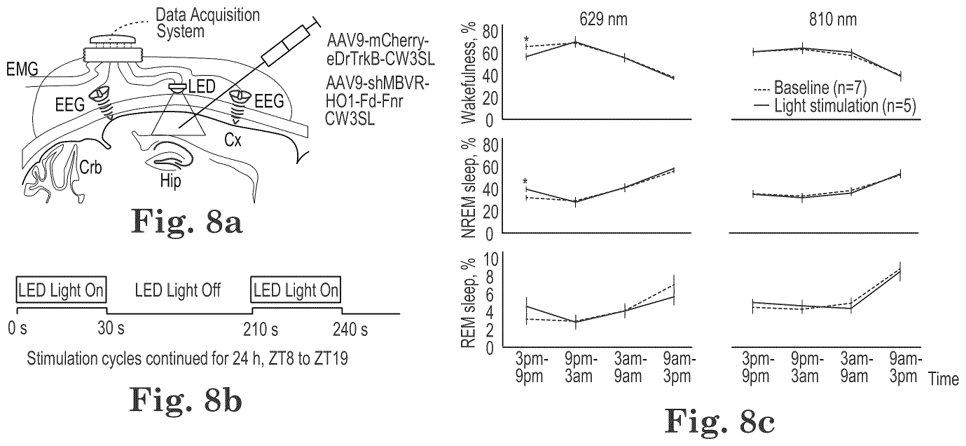

eDrTrkB was delivered into the cerebral cortex of mice by AAV9 expressing mCherry-eDrTrkB fusion under the CaMKII promoter (FIG. 8a). To increase the production of the BV chromophore in the brain, the AAV9 encoding shMBVR-HO1-Fd-Fnr was co-injected. This construct introduces HO1, Fd and Fnr genes and depletes biliverdin reductase A in the cells that together increase the BV levels. Stimulation of cells expressing eDrTrkB was achieved by exposing them to 629 nm light produced by an LED that was implanted on the skull of the mouse above the AAV-injection site (FIG. 8b). A separate group of mice was implanted with an 810 nm LED and served as a control. The mice were also implanted with EEG and EMG electrodes to study their sleep and EEG pattern.

Amounts of wakefulness, non-REM (NREM) sleep, and REM sleep were compared during the 6 hour-intervals between the baseline day and the following day during which stimulation with either 629 nm light or 810 nm light was performed. The paired-samples t-test indicated that amounts of wakefulness decreased (t(6)=2.516, p<0.05), whereas amounts of NREM sleep increased (t(6)=−2.548, p<0.05) within the first 6 h of stimulation with 629 nm light (FIG. 8c). A decrease in the average number of wake bouts (t(6)=2.538, p<0.05) and an increase in the average duration of NREM sleep bouts (t(6)=−3.136, p<0.05) during the last 6 h of exposure to 629 nm light compared to the baseline were observed. Stimulation with 810 nm light did not cause any significant changes in amounts, number of bouts, or duration of bouts of wakefulness, NREM sleep, and REM sleep (FIG. 8c).

Dependence of Sleep/Wake Amounts on eDrTrkB Expression.

The location of the AAV injection site was determined in the brain sections by analyzing the distribution of mCherry-positive cells. In the group of mice stimulated with 810 nm light (n=5), expression of mCherry was found mainly in the cerebral cortex. In some mice of this group, mCherry expression was also found in the hippocampus. A similar pattern of mCherry expression was observed in the group of mice stimulated with 629 nm light (n=7), except that two mice in this group had only a few mCherry-expressing cells in the cerebral cortex (J30 and J35). When J30 and J35 mice were excluded from the analysis, the level of statistical significance changed from the p <0.05 to p<0.01 between the baseline day and the following day during which mice were stimulated with 629 nm light (decrease in wakefulness amounts, t(4)=4.714, p<0.01; increase in NREM sleep amounts, t(4)=−4.718, p<0.01). Thus, the results strongly indicate that the cortical cells expressing mCherry-eDrTrkB were activated by FR light, and the changes seen in the wakefulness and NREM sleep were directly dependent on their activation.

Effect of eDrTrkB Activation on EEG Power Spectrum.

It has been shown that injection of BDNF into the cerebral cortex induced EEG delta power. Hence here, delta power was analyzed in the mice stimulated with 629 nm and 810 nm light. During wakefulness, mice stimulated with 629 nm light exhibited a significant increase in delta power (Z=−2.028, p<0.05) and a tendency of increase in low delta power (Z=−1.690, p<0.1) (Table 2). No differences were observed in delta power between the baseline day and the day during which mice were stimulated with 810 nm light.

TABLE 2
Cortical EEG delta power during behavioral states in mice exposed
to optogenetic light stimulation.

| | | |
|---|---|---|
| Behavior | <1.5 Hz | 0.5-4 Hz |

under 629 nm (n = 7)

| | | | | |
|---|---|---|---|---|
| Wakefulness | 20.70% ± | 8.68% ^{#} | 10.50% ± | 6.32% * |
| NREM sleep | 4.12% ± | 5.34% | 2.35% ± | 2.74% |
| REM sleep | 22.42% ± | 9.37% * | 6.79% ± | 5.99% |

under 810 nm (n = 4)

| | | | | |
|---|---|---|---|---|
| Wakefulness | −3.10% ± | 8.65% | −0.57% ± | 3.55% |
| NREM sleep | −0.65% ± | 1.31% | −0.57% ± | 0.46% |
| REM sleep | 3.32% ± | 2.11% | 0.64% ± | 0.95% |

The total mean EEG band power in <1.5 Hz (low delta) and 0.5-4 Hz (delta) during Wakefulness, NREM sleep, and REM sleep over 24 h of optogenetic stimulation was compared to the mock stimulation (no light)/baseline period from all mice in the group. The 629 nm light produced a significant increase in delta power during wakefulness, whereas 810 nm light stimulation did not produce any significant changes.
* P < 0.05.
^{#}P < 0.1.

Upregulation of cFos Expression in Neocortex by eDrTrkB.

The majority of mCherry-containing cells expressed neuronal marker NeuN, which was expected because the mCherry-eDrTrkB expression was driven by the CaMKII promoter. A marker of neuronal activity, cFos, was highly expressed in the area in the cerebral cortex injected with the AAV vector in mice stimulated with 629 nm light. The density of cFos-positive cells was especially high in mice in which cells transfected with AAVs occupied a larger area (mice #J29, J31, and J33). Interestingly, cFos expression was also seen in cells that were negative for mCherry. Additionally, cFos-positive cells were also found in the white matter underlying the cerebral cortex. When the AAV-transfected area was smaller, cFos expression was moderate (mice #J32 and J34). cFos positive cells were infrequent in the cerebral cortex of two mice stimulated with 629 nm light (#J30 and J35). cFos positive cells were also infrequently seen in all mice stimulated with 810 nm light.

The power of 629 nm light that enhanced cFos expression and changed sleep patterns was then estimated. Directly under the LED on the top of the skull, the light intensity was ˜2.5 mW mm⁻². Mouse skull reduced the FR light intensity by 24%, and 1 mm of brain tissue reduced the FR light by 53%. Therefore, it was estimated that the maximum light intensity in the brain tissue was ˜0.9 mW mm⁻² in the present studies. The intensity of 810 nm control light was similar to the intensity of 629 nm light. Directly under the LED on the top of the skull, the 810 nm light intensity was ˜3.0 mW mm⁻².

### Discussion

RTKs are a part of the family of single-pass transmembrane receptors, which includes growth hormone receptors, cytokine receptors including erythropoietin receptor, receptors of immune system, and pattern recognition receptors, like Toll-receptors. Receptors act as transmitters of an extracellular signal, for example, a chemical ligand or an interacting protein, from outside of the cell to the cytoplasm via transmembrane domains. A light photon is also such a signal, but its transmission across the mammalian plasma membrane is usually performed by the rhodopsin family of photoreceptors. This example investigated whether other types of photoreceptors, such as phytochromes, can be developed to accomplish this task.

This disclosure for the first time shows that the photosensory part of the bacterial phytochrome DrBphP retains its unique ability to reversibly photoswitch in the extracellular milieu of a mammalian cell or organ. Moreover, the light-induced conformational changes in DrBphP-PCM can be efficiently transmitted across the mammalian cell membrane via the transmembrane helix into the cytoplasm of the cell. This molecular engineering concept was effectively implemented by developing a set of eight eDrRTK optogenetic constructs from various RTK families.

Systematic optimization of the opto-RTK prototypes by changing their transmembrane domains, adding to the latter repeats of aromatic residues, varying the extracellularly targeting peptides, ER- and Golgi-export signals resulted in eDrRTKs that light-activated the downstream signaling up to 100-fold over the darkness. In the majority of eDrRTKs, the best combination of these parts consisted of the Igκ signal peptide, the original or modified transmembrane domain of HER2, and both the ER export signal and the Golgi export signals from the Kir2.1 channel.

Importantly, the resulting eDrRTKs induced the downstream signaling in mammalian non-neuronal and neuronal cells in tens of seconds. Moreover, the ability to activate eDrRTKs with FR light provides crosstalk-free spectral multiplexing with fluorescent probes and biosensors operating in the shorter spectral range, as demonstrated by co-expressing GFP-based Ca²⁺ indicator GCaMP6m. This experiment also validated the use of eDrRTKs in the so-called all-optical type of assays, in which both the disturbance of cellular metabolism and the readout of the metabolic changes are performed optically in real-time.

It was demonstrated that extracellularly targeted DrBphP-PCM represents a versatile scaffold for the development of optogenetic constructs that stay inactive in the darkness and are activated by FR light. During the opto-RTK optimization, a universal workflow was developed, which allows the conversion of other single-span transmembrane receptors into optogenetic FR constructs. Notably, the helical structure of the transmembrane domains connecting the extracellular dimeric DrBphP-PCM with the intracellular effector (catalytic) domains makes it possible to fine-tune their activity by modulating their rotational coupling in the engineered dimeric construct.

One of eDrRTKs, namely eDrTrkB, efficiently light-controlled downstream signaling and expression of immediate early genes, such as cFos, in cultured neuronal cells and in the cerebral cortex of mice, affecting animal behavior. Because a previous study demonstrated that injections of TrkB's ligand BDNF into the cerebral cortex enhanced delta power without changing sleep amounts, it was expected that stimulation of eDrTrkB expressing cortical cells would produce a similar result. However, eDrTrkB light-stimulation not only affected the EEG power spectrum but also produced an increase NREM sleep amounts, indicating that effect of the eDrTrkB light-activation in the brain was not local. The long-term activation of the TrkB pathway likely resulted in the release of neuromediators that affected the activity of nearby cells. Indeed, a high level of cFos expression was observed in both eDrTrkB expressing and non-expressing cells. Importantly, due to deep penetration of FR light, the eDrTrkB light-activation was performed non-invasively in freely moving animals.

Recent studies indicated that BDNF not only stimulates neuronal plasticity by activating TrkB and thus promoting synapse formation and supporting the maintenance of active synapses, but also mediates the elimination of inactive contacts through the activation of another receptor, p75. eDrTrkB is useful for deciphering the stimulating and eliminating effects of BDNF on neuronal plasticity via activation of either TrkB or p75 receptors. In addition, eDrTrkB is also useful for studying the TrkB role in various neuropathologies because dysfunction of the BDNF/TrkB system is involved in the development of many neurodisorders, including Alzheimer's disease, autism, and depression.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A polynucleotide encoding a chimeric polypeptide comprising:
an extracellular light-responsive polypeptide,
a transmembrane domain linked to the C-terminus of the light-responsive polypeptide, and
an intracellular domain of a receptor linked to the C-terminus of the transmembrane domain,
wherein the light-responsive polypeptide, when associated with a chromophore, is capable of switching from a first state to a second state when exposed to illumination by a wavelength, and wherein the intracellular domain of the receptor is activated at the second state.

2. The polynucleotide of claim 1, wherein the intracellular domain of the receptor dimerizes at the second state.

3. The polynucleotide of claim 1, wherein the intracellular domain of the receptor exists as an inactive dimer at the first state and exists as an active dimer at the second state.

4. The polynucleotide of any one of the preceding claims, wherein the light-responsive polypeptide comprises an N-terminal photosensory core module (PCM) of Deinococcus radiodurance bacteriophytochrome (DrBphP-PCM) or a variant thereof.

5. The polynucleotide any one of the preceding claims, wherein the light-responsive polypeptide comprises an amino acid sequence having at least 90% sequence identity to the amino acid sequence of SEQ ID NO: 2 or 3 or comprises the amino acid sequence of SEQ ID NO: 2 or 3.

6. The polynucleotide of any one of the preceding claims, wherein the receptor is a receptor tyrosine kinase (RTK).

7. The polynucleotide of claim 6, wherein the receptor tyrosine kinase is selected from EGFR, HER2, FGFR1, TrkA, TrkB, cKIT, cMet, and Insulin receptor (IR1).

8. The polynucleotide of any one of claims 6 to 7, wherein the transmembrane domain comprises a transmembrane domain of the receptor tyrosine kinase or a variant thereof.

9. The polynucleotide of claim 8, wherein the transmembrane domain comprises a transmembrane domain of EGFR, HER2, or a variant thereof.

10. The polynucleotide of any one of the preceding claims, wherein the transmembrane domain further comprises one or more repeats of Tyrosine (Y)-Phenylalanine (F).

11. The polynucleotide of any one of the preceding claims, wherein the transmembrane domain comprises an amino acid sequence having at least 90% sequence identity to the amino acid sequence of any one of SEQ ID NOs: 4-7 or comprises the amino acid sequence of any one of SEQ ID NOs: 4-7.

12. The polynucleotide of any one of the preceding claims, wherein the intracellular domain is a tyrosine kinase domain of a second receptor tyrosine kinase.

13. The polynucleotide of claim 12, wherein the second receptor tyrosine kinase is selected from EGFR, HER2, FGFR1, TrkA, TrkB, cKIT, cMet, and Insulin receptor (IR1).

14. The polynucleotide of any one of the preceding claims, wherein the intracellular domain comprises an amino acid sequence having at least 90% sequence identity to the amino acid sequence of any one of SEQ ID NOs: 8-15 or comprises the amino acid sequence of any one of SEQ ID NOs: 8-15.

15. The polynucleotide of any one of the preceding claims, wherein the chimeric polypeptide further comprises a signaling peptide linked to the N-terminus of the light-responsive polypeptide.

16. The polynucleotide of claim 15, wherein the signaling peptide comprises an Igκ signaling peptide.

17. The polynucleotide of any one of claims 15 to 16, wherein the signaling peptide comprises an amino acid sequence having at least 90% sequence identity to the amino acid sequence of SEQ ID NO: 16 or comprises the amino acid sequence of SEQ ID NO: 16

18. The polynucleotide of any one of the preceding claims, wherein the chimeric polypeptide further comprises a Golgi-export peptide linked to the C-terminus of the intracellular domain.

19. The polynucleotide of claim 18, wherein the Golgi-export peptide comprises an amino acid sequence having at least 90% sequence identity to the amino acid sequence of any one of SEQ ID NOs: 17-19 or comprises the amino acid sequence of any one of SEQ ID NOs: 17-19.

20. The polynucleotide of any one of the preceding claims, wherein: the light-responsive polypeptide is linked to the transmembrane domain via a peptide linker, or the transmembrane domain is linked to the intracellular domain via a peptide linker.

21. The polynucleotide of any one of the preceding claims, wherein the chimeric polypeptide comprises an amino acid sequence having at least 90% sequence identity to the amino acid sequence of any one of SEQ ID NOs: 20-27 or comprises the amino acid sequence of any one of SEQ ID NOs: 20-27.

22. The polynucleotide of any one of the preceding claims, wherein the wavelength is in far-red or near-infrared spectrum.

23. The polynucleotide of any one of the preceding claims, wherein the wavelength is from about 650 nm to about 900 nm.

24. The polynucleotide of claim 23, wherein the wavelength is from about 650 nm to about 700 nm.

25. The polynucleotide of claim 23, wherein the wavelength is from about 700 nm to about 780 nm.

26. A polypeptide encoded by the polynucleotide of any one of the preceding claims.

27. A vector comprising the polynucleotide of any one of claims 1 to 25.

28. The vector of claim 27 is a viral vector.

29. The vector of claim 27 or 28, wherein the vector comprises an adeno-associated viral vector, lentiviral vector, or adenoviral vector.

30. A cell comprising the polynucleotide of any one of claims 1 to 25 or the vector of claim 27.

31. A composition comprising the polynucleotide of any one of claims 1 to 25, the polypeptide of claim 26, the vector any one of claims 27 to 29, or the cell of claim 30.

32. A kit comprising the polynucleotide of any one of claims 1 to 25, the polypeptide of claim 26, the vector any one of claims 27 to 29, the cell of claim 30, or the composition of claim 31.

33. A method for modulating an expression level of a gene in a cell, comprising:
(a) introducing to the cell the polynucleotide of any one of claims 1 to 25 or the vector any one of claims 27 to 29; and exposing the cell to illumination by an activation wavelength to modulate the expression level of the gene; or
(b) providing the polypeptide of claim 26 or the cell of claim 30; and exposing the polypeptide or the cell to illumination by the activation wavelength to modulate the expression level of the gene.

34. A method for modulating an expression level of a gene in a subject, comprising: introducing to the subject the polynucleotide of any one of claims 1 to 25 or the vector any one of claims 27 to 29; and exposing the subject to illumination by an activation wavelength to modulate the expression level of the gene in the subject.

35. The method of claim 34, wherein the subject is exposed to illumination at a site of the subject where modulation of the expression level of the gene is needed.

36. The method of any of claims 33 to 35, wherein the gene is regulated by a receptor tyrosine kinase.

37. The method of any one of claims 33 to 36, wherein the activation wavelength is in far-red or near-infrared spectrum.

38. The method of any one of claims 33 to 37, wherein the wavelength is from about 650 nm to about 900 nm.

39. The method of claim 38, wherein the wavelength is from about 650 nm to about 700 nm.

40. The method of claim 38, wherein the wavelength is from about 700 nm to about 780 nm.

41. A method for identifying a modulator capable of modulating an activity or expression level of a receptor, comprising:
(a) contacting the modulator with the cell of claim 30;
(b) illuminating the cell by a wavelength;
(c) measuring the activity or expression level of the receptor in the cell and in a control cell that has not been contacted with the modulator;
(d) comparing the activity or expression level of the receptor in the cell to the activity or expression level of the receptor in the control cell; and
(e) identifying the modulator as having modulating activity for the receptor if a difference between the activity or expression level of the receptor in the cell and the activity or expression level of the receptor in the control cell is greater or less than a reference value.

42. The method of 41, wherein the receptor is a receptor tyrosine kinase (RTK).

43. The method of claim 42, wherein the receptor tyrosine kinase is selected from EGFR, HER2, FGFR1, TrkA, TrkB, cKIT, cMet, and Insulin receptor (IR1).

44. The method of any one of claims 41 to 43, wherein the modulator is an inhibitor or activator.
